(19) United States
(12) Patent Application Publication
(10) Pub. No.: US 2016/0311753 A1
(43) Pub. Date: Oct. 27, 2016

(54) Methods of Making Triacylglycerol Polyols from Fractions of Metathesized Natural Oils and Uses Thereof

(71) Applicant: Trent University, Peterborough (CA)

(72) Inventors: Suresh Narine, Peterborough (CA); Prasanth Kumar Sasidharan Pillai, Peterborough (CA); Shaojun Li, Peterborough (CA); Laziz Bouzidi, Peterborough (CA); Ali Mahdevari, Peterborough (CA)

(73) Assignee: Trent University, Peterborough (CA)

(21) Appl. No.: 15/005,522

(22) Filed: Jan. 25, 2016

Related U.S. Application Data
(60) Provisional application No. 62/107,935, filed on Jan. 26, 2015.

(51) Int. Cl. C07C 67/31; C07C 67/52; C07C 67/58; C08J 9/12; C08G 18/36; C08G 18/76; C08G 18/08; C07C 67/475; C07D 301/12

(52) U.S. Cl. CPC C07C 67/31; C07C 67/475; C07C 67/52; C07C 67/58; C07D 301/12; C08G 18/36; C08G 18/7657; C08G 18/14; C08J 9/122; C08J 2205/10; C08J 2205/06; C08J 2375/06; C08G 2101/0008

(57) ABSTRACT

Polyols derived from palm oil fractions of metathesized triacylglycerols, and their related physical properties are disclosed. Such metathesized triacylglycerol polyols are also used as a component of polyurethane applications, including polyurethane foams.

### CROSS-REFERENCE TO RELATED APPLICATIONS

The present application claims the benefit of priority of U.S. Provisional Application No. 62/107,935, filed Jan. 26, 2015, which is hereby incorporated by reference as though set forth herein in its entirety.

### TECHNICAL FIELD

This application relates to polyols from the fractions of metathesized triacylglycerols and their related physical and thermal properties. Such polyols from the fractions of metathesized triacylglycerols are also used as a component in polyurethane applications, including polyurethane foams.

### DESCRIPTION OF RELATED ART

Polyurethanes are one of the most versatile polymeric materials with regards to both processing methods and mechanical properties. Polyurethanes are formed either based on the reaction of NCO groups and hydroxyl groups, or via non-isocyanate pathways, such as the reaction of cyclic carbonates with amines, self-polycondensation of hydroxyl-acyl azides or melt transurethane methods. The most common method of urethane production is via the reaction of a polyol and an isocyanate which forms the backbone urethane group. Cross-linking agents, chain extenders, blowing agents and other additives may also be added as needed. The proper selection of reactants enables a wide range of polyurethane elastomers, sheets, foams, and the like.

Traditionally, petroleum-derived polyols have been widely used in the manufacturing of polyurethane foams. However, there has been an increased interest in the use of renewable resources in the manufacturing of polyurethane foams. This has led to research into developing natural oil-based polyols for use in the manufacturing of foams. The present effort details the synthesis of natural oil (palm oil, for example) based fractions of metathesized triacylglycerol and polyols thereof, which may be used in polyurethane applications, such as rigid and flexible polyurethane foams. The present effort also discloses physical and thermal properties of such polyols, and the formulation of polyurethane applications (such as foams) using such polyols as a component.

### SUMMARY

In a first aspect, the disclosure provides methods of making a triacylglycerol polyol from palm oil, the method comprising: providing a metathesized triacylglycerol composition, which is formed by the cross-metathesis of a natural oil with lower-weight olefins, and which comprises triglyceride compounds having one or more carbon-carbon double bonds; separating a fraction of the metathesized triacylglycerol composition to form a fractionated metathesized triacylglycerol composition, which comprises compounds having one or more carbon-carbon double bonds; and reacting at least a portion of the carbon-carbon double bonds in the compounds comprised by the fractionated metathesized triacylglycerol composition to form a triacylglycerol polyol composition.

In a second aspect, the disclosure provides methods of forming a polyurethane composition, comprising: providing a triacylglycerol polyol and an organic diisocyanate, wherein providing the triacylglycerol polyol comprises making a triacylglycerol polyol according to the first aspect or any embodiments thereof; and reacting the triacylglycerol polyol and the organic diisocyanate to form a polyurethane composition. In some embodiments, the polyurethane composition is a polyurethane foam.

Further aspects and embodiments of the present disclosure are set forth in the Detailed Description.

### BRIEF DESCRIPTION OF THE DRAWINGS

The following drawings are provided for purposes of illustrating various embodiments of the compounds, compositions, and methods disclosed herein. The drawings are provided for illustrative purposes only, and are not intended to describe any preferred compounds, preferred compositions, or preferred methods, or to serve as a source of any limitations on the scope of the claimed inventions.

FIG. 1: FIG. 1A depicts the DSC thermograms of MTAG of palm oil cooling (0.1° C./min); FIG. 1B depicts the DSC thermograms of MTAG of palm oil subsequent heating (5° C./min).

FIG. 2: FIG. 2A depicts DSC thermograms of PMTAG fractions obtained by dry fractionation—rates method (D1), during cooling (5° C./min) of liquid fractions; FIG. 2B depicts DSC thermograms of PMTAG fractions obtained by dry fractionation—rates method (D1), during cooling (5° C./min) of solid fractions; FIG. 2C depicts DSC thermograms of PMTAG fractions obtained by dry fractionation—rates method (D1), during subsequent heating (5° C./min) of liquid fractions; FIG. 2D depicts DSC thermograms of PMTAG fractions obtained by dry fractionation—rates method (D1), during subsequent heating (5° C./min) of solid fractions. (Note: For FIGS. 2A-2D, numbers 1 to 4 refer to the different experiments listed in Table 5. SFi, and LFi, i=1-4: Solid fraction and Liquid fraction of i^{th} experiment, respectively.)

FIG. 3: FIG. 3A depicts DSC thermograms of the fractions of PMTAG obtained by dry fractionation—quiescent method (D2), during cooling (5° C./min) of liquid fractions; FIG. 3B depicts DSC thermograms of the fractions of PMTAG obtained by dry fractionation—quiescent method (D2), during cooling (5° C./min) of solid fractions; FIG. 3C depicts DSC thermograms of the fractions of PMTAG obtained by dry fractionation—quiescent method (D2), during subsequent heating (5° C./min) of liquid fractions; FIG. 3D depicts DSC thermograms of the fractions of PMTAG obtained by dry fractionation—quiescent method (D2) at subsequent heating (5° C./min) of solid fractions. (Note: For FIGS. 3A-3D, T_{On} (° C.): onset temperature of crystallization. Numbers 1 to 4 refer to the different experiments listed in Table 6. SFi, and LFi, i=1-4: Solid fraction and Liquid fraction of i^{th} experiment, respectively.)

FIG. 4: FIG. 4A-4C depicts ¹H-NMR of SF-PMTAG; FIG. 4D-4F depicts ¹H-NMR of LF-PMTAG.

FIG. 5: FIGS. 5A-5C depicts HPLC of SF-PMTAG; FIGS. 5D-5F depicts HPLC of LF-PMTAG; FIG. 5G depicts HPLC of PMTAG.

FIG. 6: FIG. 6A-6D depicts TGA and DTG curves of PMTAG fractions obtained for solid fraction (SF-PMTAG); FIG. 6E-H depicts TGA and DTG curves of PMTAG fractions obtained for liquid fraction (LF-PMTAG). (Note: for FIGS. 6A-6H, (D1): dry crystallization—rates method, (D2): dry crystallization—quiescent method, and (S): solvent aided crystallization method).

FIG. 7: FIGS. 7A-7C depicts DSC thermograms of the standard liquid and solid fractions of PMTAG fractions obtained by dry crystallization (rates method (D1) and quiescent method (D2)) and solvent aided crystallization method (S), during cooling (5° C./min); FIGS. 7D-7F depicts DSC thermograms of the standard liquid and solid fractions of PMTAG fractions obtained by dry crystallization (rates method (D1) and quiescent method (D2)) and solvent aided crystallization method (S), during subsequent heating (5° C./min).

FIG. 8: FIG. 8A-8B depicts DSC cooling thermograms (at 5° C./min) of the standard liquid and solid fractions of PMTAG compared. Dry crystallization (rates method (D1) and quiescent method (D2)) and solvent aided crystallization method (S); FIG. 8C-8D depicts DSC heating thermograms (at 5° C./min) of the standard liquid and solid fractions of PMTAG compared. Dry crystallization (rates method (D1) and quiescent method (D2)) and solvent aided crystallization method (S).

FIG. 9: FIGS. 9A-9C depicts SFC versus temperature of SF-PMTAG and LF-PMTAG, during cooling (5° C./min); FIG. 9D-9F depicts SFC versus temperature of SF-PMTAG and LF-PMTAG, during subsequent heating (5° C./min). (Note: For FIGS. 9A-9F, 1. SF(D1)-PMTAG and LF(D1)-PMTAG; 2. SF(D2)-PMTAG and LF(D2)-PMTAG; 3. SF(S)-PMTAG; LF(S)-PMTAG.)

FIG. 10: FIGS. 10A-10B depicts SFC versus temperature of SF-PMTAG and LF-PMTAG, during cooling (5° C./min); FIGS. 10C-10D depicts SFC versus temperature of SF-PMTAG and LF-PMTAG, during subsequent heating (5° C./min). (Note: For FIGS. 10A-10D, 1. SF-PMTAG and 2. LF-PMTAG.)

FIG. 11: FIGS. 11A-C depicts shear rate versus shear stress curves of the fractions of palm oil MTAG obtained at selected temperatures of liquid fraction (LF-PMTAG); FIGS. 11D-F depicts shear rate versus shear stress curves of the fractions of palm oil MTAG obtained at selected temperatures of solid fraction (SF-PMTAG).

FIG. 12: FIGS. 12A, 12E, and 12I depicts viscosity versus temperature curves obtained during cooling of PMTAG fractions of liquid fractions; FIGS. 12B, 12F, and 12J depicts viscosity versus temperature curves obtained during cooling of PMTAG fractions of solid fractions; FIGS. 12C, 12G, and 12K depicts viscosity versus temperature curves obtained during cooling of PMTAG fractions of liquid and solid fractions combined; FIGS. 12D, 12H, and 12L depicts viscosity difference (Δη) between the solid and liquid fractions versus temperature curves. (Note: For FIGS. 12A-12L, (a1-d1) LF(D1)-PMTAG and SF(D1)-PMTAG, (a2-d2) LF(D2)-PMTAG and SF(D2)-PMTAG and (a3-d3) LF(S)-PMTAG and SF(S)-PMTAG.)

FIG. 13: FIG. 13A depicts viscosity versus temperature curves obtained during cooling of PMTAG fractions of liquid fractions compared; FIG. 13B depicts viscosity versus temperature curves obtained during cooling of PMTAG fractions of solid fractions compared; FIG. 13C depicts viscosity versus temperature curve difference (Δη/(LF)) between LF(D1) and LF(S); FIG. 13D depicts viscosity versus temperature curve difference (Δη(SF)) between SF(D1) and SF(S).

FIG. 14: FIGS. 14A-14C depicts ¹H-NMR spectrum of epoxy LF-PMTAG; FIGS. 14D-14F depicts ¹H-NMR spectrum of epoxy SF-PMTAG. (Note: For FIGS. 14A-14F, (a1-b1) LF(D1)-PMTAG and SF(D1)-PMTAG, (a2-b2) LF(D2)-PMTAG and SF(D2)-PMTAG and (a3-b3) LF(S)-PMTAG and SF(S)-PMTAG.)

FIG. 15: FIG. 15A depicts ¹H-NMR spectrum of LF(D1)-PMTAG Polyol; FIG. 15B depicts ¹H-NMR spectrum of LF(D2)-PMTAG Polyol; FIG. 15C depicts ¹H-NMR spectrum of LF(S)-PMTAG Polyol.

FIG. 16: FIG. 16A depicts ¹H-NMR spectrum of SF(D1)-PMTAG Polyol; FIG. 16B depicts ¹H-NMR spectrum of SF(D2)-PMTAG Polyol; FIG. 16C depicts ¹H-NMR spectrum of SF(S)-PMTAG Polyol.

FIG. 17: FIG. 17A depicts HPLC of LF(D1)-PMTAG Polyol; FIG. 17B depicts HPLC of LF(D2)-PMTAG Polyol; FIG. 17C depicts HPLC of LF(S)-PMTAG Polyol.

FIG. 18: FIG. 18A depicts HPLC of SF(D1)-PMTAG Polyol; FIG. 18B depicts HPLC of SF(D2)-PMTAG Polyol; FIG. 18C depicts HPLC of SF(S)-PMTAG Polyol.

FIG. 19: FIG. 19A depicts HPLC of PMTAG Polyol; FIG. 19B depicts HPLC of PMTAG Green Polyol.

FIG. 20: FIG. 20A depicts TGA and DTG profiles of (a) LF(D1)-PMTAG Polyol; FIG. 20B depicts TGA and DTG profiles of LF(S)-PMTAG Polyol; FIG. 20C depicts TGA and DTG profiles of LF(D2)-PMTAG Polyol; FIG. 20D depicts DTG profiles of LF(D1, D2 and S)-PMTAG Polyols.

FIG. 21: FIG. 21A depicts TGA and DTG profiles of SF(D1)-PMTAG Polyol; FIG. 21B depicts TGA and DTG profiles of SF(S)-PMTAG Polyol; FIG. 21C depicts TGA and DTG profiles of SF(D2)-PMTAG Polyol; FIG. 21D depicts DTG profiles of SF-PMTAG Polyols.

FIG. 22: FIG. 22A depicts DSC thermograms of polyols obtained from the liquid fractions of PMTAG during cooling (5° C./min); FIG. 22B depicts DSC thermograms of polyols obtained from the liquid fractions of PMTAG during subsequent heating (5° C./min). (Note: For FIGS. 22A and 22B, Curve LF(D1): LF(D1)-PMTAG Polyol; curve LF(S): LF(S)-PMTAG Polyol; and curve LF(D2): LF(D2)-PMTAG Polyol.)

FIG. 23: FIG. 23A depicts DSC thermograms of Polyols obtained from the solid fractions of PMTAG during cooling (5.0° C./min); FIG. 23B depicts DSC thermograms of Polyols obtained from the solid fractions of PMTAG during subsequent heating (5° C./min). (Note: In FIGS. 23A and 23B, Curve SF(D1): SF(D1)-PMTAG Polyol; and curve SF(S): SF(S)-PMTAG Polyol.)

FIG. 24: FIG. 24A depicts SFC versus temperature of polyols from PMTAG liquid fractions cooling during 5° C./min; FIG. 24B depicts SFC versus temperature of polyols from PMTAG liquid fractions subsequent heating during 5° C./min. (Note: In FIGS. 24A and 24B, LF(S)-PMTAG Polyol and LF(D1)-PMTAG Polyol: polyols from the liquid fractions of PMTAG obtained by solvent and dry fractionation of PMTAG, respectively.)

FIG. 25: FIG. 25A depicts SFC versus temperature of PMTAG solid fractions of Polyols obtained from the solid fractions of PMTAG during cooling (5.0° C./min); FIG. 25B depicts SFC versus temperature of PMTAG solid fractions of Polyols obtained from the solid fractions of PMTAG during subsequent heating (5° C./min). (Note: In FIGS. 25A and 25B, Curve SF(D1): SF(D1)-PMTAG Polyol; and curve SF(S): SF(S)-PMTAG Polyol.)

FIG. 26: FIG. 26A depicts shear rate-shear stress of LF(D1)-PMTAG Polyol; FIG. 26B depicts shear rate-shear stress of LF(D2)-PMTAG Polyol; FIG. 26C depicts shear rate-shear stress of LF(S)-PMTAG Polyol.

FIG. 27: FIG. 27A depicts viscosity versus temperature curves obtained during cooling (1° C./min) of LF(D1)-PMTAG Polyol; FIG. 27B depicts viscosity versus temperature curves obtained during cooling (1° C./min) of LF(D2)-PMTAG Polyol; FIG. 27C depicts viscosity versus temperature curves obtained during cooling (1° C./min) of LF(S)-PMTAG Polyol; FIG. 27D depicts viscosity of LF(S)-, LF(D1)- and LF(D2)-PMTAG Polyols compared.

FIG. 28: FIG. 28A depicts shear rate-shear stress of SF(D1)-PMTAG Polyol; FIG. 28B depicts shear rate-shear stress of SF(D2)-PMTAG Polyol; FIG. 28C depicts shear rate-shear stress of SF(S)-PMTAG Polyol.

FIG. 29: FIG. 29A depicts viscosity versus temperature curves obtained during cooling (1° C./min) of SF(D1)-PMTAG Polyol; FIG. 29B depicts viscosity versus temperature curves obtained during cooling (1° C./min) of SF(D2)-PMTAG Polyol; FIG. 29C depicts viscosity versus temperature curves obtained during cooling (1° C./min) of SF(S)-PMTAG Polyol; FIG. 29D depicts viscosity of SF(S)-, SF(D1)- and SF(D2)-PMTAG Polyols compared.

FIG. 30: FIG. 30A depicts a comparison between the viscosities of SF(S)-PMTAG Polyols; FIG. 30B depicts a comparison between the viscosities of LF-PMTAG Polyols.

FIG. 31 depicts ¹H-NMR spectrum of crude MDI.

FIG. 32: FIGS. 32A-32B depicts SEM micrographs of rigid LF(D1)-MTAG Polyol Foam; FIGS. 32C-32D depicts SEM micrographs of rigid LF(D2)-MTAG Polyol Foam; FIGS. 32E-32F depicts SEM micrographs of rigid LF(S)-MTAG Polyol Foam. (Note: In FIGS. 32A-32F, 1. SEM magnification 51× and 2. SEM magnification 102×.)

FIG. 33: FIGS. 33A-33B depicts SEM micrographs of flexible LF(D1)-MTAG Polyol Foam; FIG. 33C-33D depicts SEM micrographs of flexible LF(D2)-MTAG Polyol Foam; FIG. 33E-33F depicts SEM micrographs of flexible LF(S)-MTAG Polyol Foam. (Note: In FIGS. 33A-33F, 1. SEM magnification 51× and 2. SEM magnification 102×.)

FIG. 34: FIG. 34A depicts FTIR spectra of rigid LF-PMTAG Polyol foams; FIG. 34B depicts FTIR spectra of flexible LF-PMTAG Polyol foams. (Note: In FIGS. 34A and 34B, LF(D1): LF(D1)-MTAG Polyol Foam; LF(D2): LF(D2)-PMTAG Polyol foams; LF(S): LF(S)-MTAG Polyol Foam).

FIG. 35: FIG. 35A depicts DTG curves of rigid LF-PMTAG Polyol foams; FIG. 35B depicts DTG curves of flexible LF-PMTAG Polyol foams. (Note: In FIGS. 35A and 35B, LF(D1): LF(D1)-MTAG Polyol Foams; LF(D2): LF(D2)-PMTAG Polyol Foams; LF(S): LF(S)-PMTAG Polyol Foams).

FIG. 36: FIG. 36A depicts 2^{nd} heating DSC thermogram of LF-PMTAG Polyol Foams of rigid foams; FIG. 36B depicts 2^{nd} heating DSC thermogram of LF-PMTAG Polyol Foams of flexible foams. (Note: In FIGS. 36A and 36B, Rigid and Flexible polyol foams have a density of 166 kg/m³ and 155 kg/m³, respectively.)

FIG. 37 depicts stress versus strain curves of rigid foams. (Note: For FIG. 37, LF(D1)-RF163: LF(D1) Rigid LF(D1)-PMTAG Polyol Foam with density=163 kgm⁻³; LF(D2)-RF167: Rigid LF(D2)-PMTAG Polyol Foam with density=167 kgm⁻³; LF(S)-RF166: Rigid LF(S)-PMTAG Polyol Foam with density=166 kgm⁻³.)

FIG. 38 depicts stress versus strain curves of flexible foams. (Note: In FIG. 38, LF(D1)-FF160: Flexible LF(D1)-PMTAG Polyol Foam with density=160 kgm⁻³; LF(D2)-FF155: Flexible LF(D2)-PMTAG Polyol Foam with density=155 kgm⁻³; LF(S)-FF166: Flexible LF(S)-PMTAG Polyol Foam with density=166 kgm⁻³.)

FIG. 39 depicts % Recovery of flexible LF-PMTAG Polyol foams as a function of time. (Note: In FIG. 39, LF(D1)-FF160: Flexible LF(D1)-PMTAG Polyol Foam with density=160 kgm⁻³; LF(D2)-FF160: Flexible LF(D2)-PMTAG Polyol Foam with density=166 kgm⁻³; LF(S)-FF155: Flexible LF(S)-PMTAG Polyol Foam with density=155 kgm⁻³.)

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Nomenclature and Acronyms

To simplify the presentation and discussion of the data of the present patent application, a comprehensive nomenclature of the different compounds and acronyms used herein is presented in Table 1.

TABLE 1

| | |
|---|---|
| Name | Acronym |

Metathesized Triacylglycerol

| | |
|---|---|
| Metathesized Triacylglycerol | MTAG |
| MTAG of Palm Oil | PMTAG |
| Solid Fraction | SF |
| Liquid Fraction | LF |
| Solid Fraction of PMTAG | SF-PMTAG |
| Liquid Fraction of PMTAG | LF-PMTAG |
| Solid Fraction of PMTAG from Dry Fractionation - | SF(D1)-PMTAG |
| Rates Method (D1) | |
| Liquid Fraction of PMTAG from Dry Fractionation- | LF(D1)-PMTAG |
| Rates Method (D1) | |
| Solid Fraction of PMTAG from Dry Fractionation - | SF(D2)-PMTAG |
| Quiescent Method (D2) | |
| Liquid Fraction of PMTAG from Dry Fractionation- | LF(D2)-PMTAG |
| Quiescent Method (D2) | |
| Solid Fraction of PMTAG from Solvent Fractionation | SF(S)-PMTAG |
| (S) | |
| Liquid Fraction of PMTAG from Solvent Fractionation | LF(S)-PMTAG |
| (S) | |

Polyols

| | |
|---|---|
| Epoxy of Solid Fraction of PMTAG | Epoxy |
| | SF-PMTAG |
| Epoxy of Liquid Fraction of PMTAG | Epoxy |
| | LF-PMTAG |
| Polyol from the Solid Fraction of PMTAG from Dry | SF(D1)-PMTAG |
| Fractionation- Rates Method (D1) | Polyol |
| Polyol from the Liquid Fraction of PMTAG from Dry | LF(D1)-PMTAG |
| Fractionation- Rates Method (D1) | Polyol |
| Polyol from the Solid Fraction of PMTAG from Dry | SF(D2)-PMTAG |
| Fractionation- Quiescent Method (D2) | Polyol |
| Polyol from the Liquid Fraction of PMTAG from Dry | LF(D2)-PMTAG |
| Fractionation- Quiescent Method (D2) | Polyol |
| Polyol from the Solid Fraction of PMTAG from | SF(S)-PMTAG |
| Solvent Fractionation (S) | Polyol |
| Polyol from the Liquid Fraction of PMTAG from | LF(S)-PMTAG |
| Solvent Fractionation (S) | Polyol |

Foams

| | |
|---|---|
| Rigid Foam | RF |
| Flexible Foam | FF |
| Foam from Polyol from the Liquid Fraction of | LF(D1)-PMTAG |
| PMTAG from Dry Fractionation- Rates Method (D1) | Polyol Foam |
| Foam from Polyol from the Liquid Fraction of | LF(D2)-PMTAG |
| PMTAG from Dry Fractionation- Quiescent | Polyol Foam |
| Method (D2) | |
| Foam from Polyol from the Liquid Fraction of | LF(S)-PMTAG |
| PMTAG from Solvent Fractionation (S) | Polyol Foam |
| Rigid Foam having a density of xxx kg/m³ from Polyol | LF(D1)-RFxxx |
| of the Liquid Fraction obtained by Dry fractionation of | |
| PMTAG - Rates Method (D1) | |
| Rigid Foam having a density of xxx kg/m³ from Polyol | LF(D2)-RFxxx |
| of the Liquid Fraction obtained by Dry fractionation of | |
| PMTAG - Quiescent Method (D2) | |
| Rigid Foam having a density of xxx kg/m³ from Polyol | LF(S)-RFxxx |
| of the Liquid Fraction obtained by Solvent | |
| fractionation of PMTAG (S) | |
| Flexible Foam having a density of xxx kg/m³ from | LF(D1)-FFxxx |
| Polyol of the Liquid Fraction obtained by Dry | |
| fractionation of PMTAG - Rates Method (D1) | |
| Flexible Foam having a density of xxx kg/m³ from | LF(D2)-FFxxx |
| Polyol of the Liquid Fraction obtained by Dry | |
| fractionation of PMTAG - Quiescent Method (D2) | |
| Flexible Foam having a density of xxx kg/m³ from | LF(S)-FFxxx |
| Polyol of the Liquid Fraction obtained by Solvent | |
| fractionation of PMTAG (S) | |

### Metathesized Triacylglycerols of Palm Oil (PMTAG)

### Synthesis of Metathesized Triacylglycerols for Production of Polyols

The synthesis of rigid and flexible polyurethane foams and other polyurethanes from natural oil based metathesized triacylglycerol (MTAG) and polyols thereof, begins with the initial synthesis of the MTAGs themselves. A general definition of a metathesized triacylglycerol is the product formed from the metathesis reaction (self-metathesis or cross-metathesis) of an unsaturated triglyceride in the presence of a metathesis catalyst to form a product comprising one or more metathesis monomers, oligomers or polymers.

Metathesis is a catalytic reaction that involves the interchange of alkylidene units among compounds containing one or more double bonds (i.e., olefinic compounds) via the formation and cleavage of the carbon-carbon double bonds. The metathesis catalyst in this reaction may include any catalyst or catalyst system that catalyzes a metathesis reaction. Generally, cross metathesis may be represented schematically as shown in Scheme 1 below:

R¹—CH═CH—R²+R³—CH═CH—R⁴ [symbol] R¹—CH═CH—R³+R¹—CH═CH—R⁴+R²—CH═CH—R³+R²—CH═CH—R⁴+R¹—CH═CH—R¹+R²—CH═CH—R²+R³—CH═CH—R³+R⁴—CH═CH—R⁴

Scheme 1. Representation of Cross-Metathesis Reaction. Wherein R¹, R², R³, and R⁴ are Organic Groups

Suitable homogeneous metathesis catalysts include combinations of a transition metal halide or oxo-halide (e.g., WOCl₄ or WCl₆) with an alkylating cocatalyst (e.g., Me₄Sn). Preferred homogeneous catalysts are well-defined alkylidene (or carbene) complexes of transition metals, particularly Ru, Mo, or W. These include first and second-generation Grubbs catalysts, Grubbs-Hoveyda catalysts, and the like. Suitable alkylidene catalysts have the general structure:

M[X¹X²L¹L²(L³)ₙ]═Cₘ═C(R¹)R²

where M is a Group 8 transition metal, L¹, L², and L³ are neutral electron donor ligands, n is 0 (such that L³ may not be present) or 1, m is 0, 1, or 2, X¹ and X² are anionic ligands, and R¹ and R² are independently selected from H, hydrocarbyl, substituted hydrocarbyl, heteroatom-containing hydrocarbyl, substituted heteroatom-containing hydrocarbyl, and functional groups. Any two or more of X¹, X², L¹, L², L³, R¹ and R² can form a cyclic group and any one of those groups can be attached to a support.

First-generation Grubbs catalysts fall into this category where m=n=0 and particular selections are made for n, X¹, X², L¹, L², L³, R¹ and R² as described in U.S. Pat. Appl. Publ. No. 2010/0145086 (“the '086 publication”), the teachings of which related to all metathesis catalysts are incorporated herein by reference. Second-generation Grubbs catalysts also have the general formula described above, but L¹ is a carbene ligand where the carbene carbon is flanked by N, O, S, or P atoms, preferably by two N atoms. Usually, the carbene ligand is part of a cyclic group. Examples of suitable second-generation Grubbs catalysts also appear in the '086 publication.

In another class of suitable alkylidene catalysts, L¹ is a strongly coordinating neutral electron donor as in first- and second-generation Grubbs catalysts, and L² and L³ are weakly coordinating neutral electron donor ligands in the form of optionally substituted heterocyclic groups. Thus, L² and L³ are pyridine, pyrimidine, pyrrole, quinoline, thiophene, or the like. In yet another class of suitable alkylidene catalysts, a pair of substituents is used to form a bi- or tridentate ligand, such as a biphosphine, dialkoxide, or alkyldiketonate. Grubbs-Hoveyda catalysts are a subset of this type of catalyst in which L² and R² are linked. Typically, a neutral oxygen or nitrogen coordinates to the metal while also being bonded to a carbon that is α-, β-, or γ- with respect to the carbene carbon to provide the bidentate ligand. Examples of suitable Grubbs-Hoveyda catalysts appear in the '086 publication.

The structures below (Scheme 2) provide just a few illustrations of suitable catalysts that may be used:

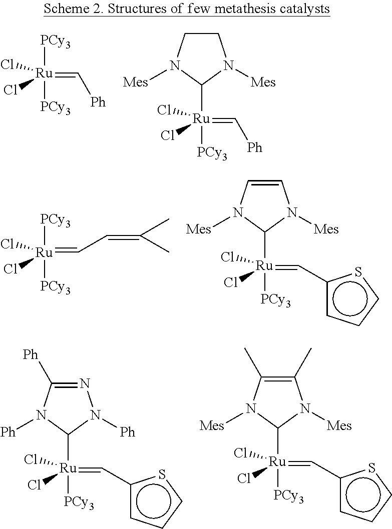

Heterogeneous catalysts suitable for use in the self- or cross-metathesis reactions include certain rhenium and molybdenum compounds as described, e.g., by J. C. Mol in Green Chem. 4 (2002) 5 at pp. 11-12. Particular examples are catalyst systems that include Re₂O₇ on alumina promoted by an alkylating cocatalyst such as a tetraalkyl tin lead, germanium, or silicon compound. Others include MoCl₃ or MoCl₅ on silica activated by tetraalkyltins. For additional examples of suitable catalysts for self- or cross-metathesis, see U.S. Pat. No. 4,545,941, the teachings of which are incorporated herein by reference, and references cited therein. See also J. Org. Chem. 46 (1981) 1821; J. Catal. 30 (1973) 118; Appl. Catal. 70 (1991) 295; Organometallics 13 (1994) 635; Olefin Metathesis and Metathesis Polymerization by Ivin and Mol (1997), and Chem. & Eng. News 80(51), Dec. 23, 2002, p. 29, which also disclose useful metathesis catalysts. Illustrative examples of suitable catalysts include ruthenium and osmium carbene catalysts as disclosed in U.S. Pat. Nos. 5,312,940, 5,342,909, 5,710,298, 5,728,785, 5,728,917, 5,750,815, 5,831,108, 5,922,863, 6,306,988, 6,414,097, 6,696,597, 6,794,534, 7,102,047, 7,378,528, and U.S. Pat. Appl. Publ. No. 2009/0264672 A1, and PCT/US2008/009635, pp. 18-47, all of which are incorporated herein by reference. A number of metathesis catalysts that may be advantageously employed in metathesis reactions are manufactured and sold by Materia, Inc. (Pasadena, Calif.).

As a non-limiting aspect, a typical route to obtain MTAG is via the cross metathesis of a natural oil with a lower weight olefin. As a non-limiting aspect, reaction routes using triolein with 1,2-butene and triolein with ethylene are shown below in Schemes 3a and 3b, respectively.

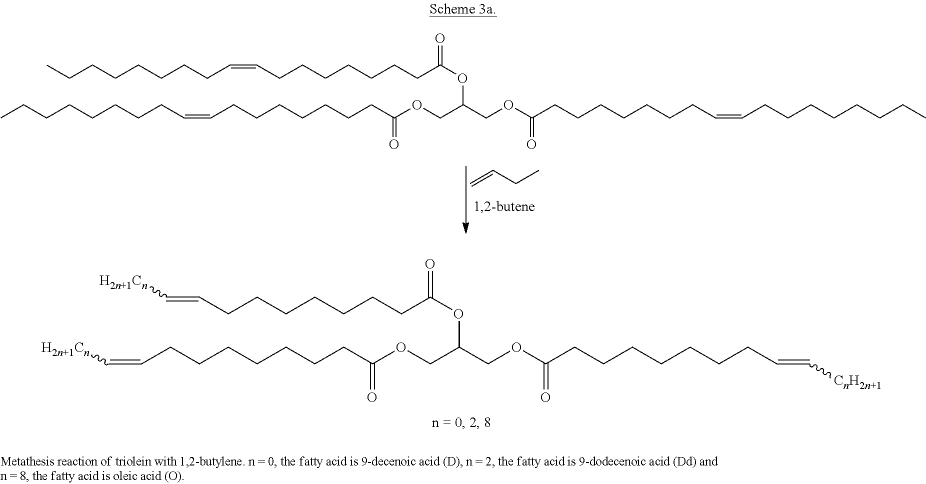

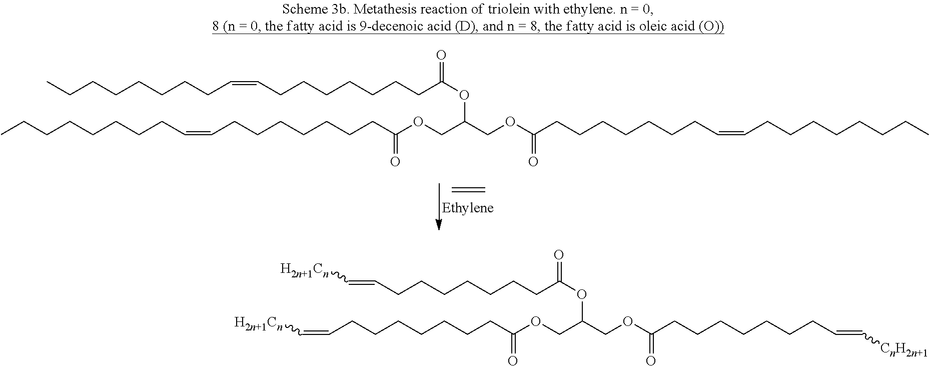

As used herein, the term “lower weight olefin” may refer to any one or a combination of unsaturated straight, branched, or cyclic hydrocarbons in the C₂ to C₁₄ range. Lower weight olefins include “alpha-olefins” or “terminal olefins,” wherein the unsaturated carbon-carbon bond is present at one end of the compound. Lower weight olefins may also include dienes or trienes. Examples of low weight olefins in the C₂ to C₆ range include, but are not limited to: ethylene, propylene, 1-butene, 2-butene, isobutene, 1-pentene, 2-pentene, 3-pentene, 2-methyl-1-butene, 2-methyl-2-butene, 3-methyl-1-butene, cyclopentene, 1-hexene, 2-hexene, 3-hexene, 4-hexene, 2-methyl-1-pentene, 3-methyl-1-pentene, 4-methyl-1-pentene, 2-methyl-2-pentene, 3-methyl-2-pentene, 4-methyl-2-pentene, 2-methyl-3-pentene, and cyclohexene. Other possible low weight olefins include styrene and vinyl cyclohexane. In certain embodiments, it is preferable to use a mixture of olefins, the mixture comprising linear and branched low weight olefins in the C₄-C₁₀ range. In one embodiment, it may be preferable to use a mixture of linear and branched C₄ olefins (i.e., combinations of: 1-butene, 2-butene, and/or isobutene). In other embodiments, a higher range of C₁₁-C₁₄ may be used.

As used herein, the term “natural oil” may refer to oil derived from plants or animal sources. The term “natural oil” includes natural oil derivatives, unless otherwise indicated. Examples of natural oils include, but are not limited to, vegetable oils, algal oils, animal fats, tall oils, derivatives of these oils, combinations of any of these oils, and the like. Representative non-limiting examples of vegetable oils include canola oil, rapeseed oil, coconut oil, corn oil, cottonseed oil, jojoba oil, olive oil, palm oil, peanut oil, safflower oil, sesame oil, soybean oil, sunflower oil, linseed oil, palm kernel oil, tung oil, jatropha oil, mustard oil, camelina oil, pennycress oil, hemp oil, algal oil, and castor oil. Representative non-limiting examples of animal fats include lard, tallow, poultry fat, yellow grease, and fish oil. Tall oils are by-products of wood pulp manufacture. In certain embodiments, the natural oil may be refined, bleached, and/or deodorized. In some embodiments, the natural oil may be partially or fully hydrogenated. In some embodiments, the natural oil is present individually or as mixtures thereof.

Natural oils generally comprise triacylglycerols of saturated and unsaturated fatty acids. Suitable fatty acids may be saturated or unsaturated (monounsaturated or polyunsaturated) fatty acids, and may have carbon chain lengths of 3 to 36 carbon atoms. Such saturated or unsaturated fatty acids may be aliphatic, aromatic, saturated, unsaturated, straight chain or branched, substituted or unsubstituted and mono-, di-, tri-, and/or poly-acid variants, hydroxy-substituted variants, aliphatic, cyclic, alicyclic, aromatic, branched, aliphatic- and alicyclic-substituted aromatic, aromatic-substituted aliphatic and alicyclic groups, and heteroatom substituted variants thereof. Any unsaturation may be present at any suitable isomer position along the carbon chain as would be obvious to a person skilled in the art.

Some non-limiting examples of saturated fatty acids include propionic, butyric, valeric, caproic, enanthic, caprylic, pelargonic, capric, undecylic, lauric, tridecylic, myristic, pentadecanoic, palmitic, margaric, stearic, nonadecyclic, arachidic, heneicosylic, behenic, tricosylic, lignoceric, pentacoyslic, cerotic, heptacosylic, carboceric, montanic, nonacosylic, melissic, lacceroic, psyllic, geddic, ceroplastic acids.

Some non-limiting examples of unsaturated fatty acids include butenoic, pentenoic, hexenoic, pentenoic, octenoic, nonenoic acid, decenoic acid, undecenoic acid, dodecenoic acid, tridecenoic, tetradecenoic, pentadecenoic, palmitoleic, palmitelaidic, oleic, ricinoleic, vaccenic, linoleic, linolenic, elaidic, eicosapentaenoic, behenic and erucic acids. Some unsaturated fatty acids may be monounsaturated, diunsaturated, triunsaturated, tetraunsaturated or otherwise polyunsaturated, including any omega unsaturated fatty acids.

In a typical triacylglycerol, each of the carbons in the triacylglycerol molecule is numbered using the stereospecific numbering (sn) system. Thus one fatty acyl chain group is attached to the first carbon (the sn-1 position), another fatty acyl chain is attached to the second, or middle carbon (the sn-2 position), and the final fatty acyl chain is attached to the third carbon (the sn-3 position). The triacylglycerols described herein may include saturated and/or unsaturated fatty acids present at the sn-1, sn-2, and/or sn-3 position

In some embodiments, the natural oil is a palm oil. Palm oil is typically a semi-solid at room temperature and comprises approximately 50% saturated fatty acids and approximately 50% unsaturated fatty acids. Palm oil typically comprises predominately fatty acid triacylglycerols, although monoacylglycerols and diacylglycerols may also be present in small amounts. The fatty acids typically have chain lengths ranging from about C12 to about C20. Representative saturated fatty acids include, for example, C12:0, C14:0, C16:0, C18:0, and C20:0 saturated fatty acids. Representative unsaturated fatty acids include, for example, C16:1, C18:1, C18:2, and C18:3 unsaturated fatty acids. As used herein, metathesized triacylglycerols derived from palm oil may be referred to as PMTAG.

Palm oil is constituted mainly of palmitic acid and oleic acid with ˜43% and ˜41%, respectively. The fatty acid and triacylglycerol (TAG) profiles of palm oil are listed in Table 2 and Table 3, respectively.

TABLE 2
Fatty acid profile of palm oil

| Fatty Acid | C12:0 | C14:0 | C16:0 | C18:0 | C18:1 | C18:2 | Others |
|---|---|---|---|---|---|---|---|
| Content (%) | 0.2 | 1.0 | 42.9 | 4.4 | 40.8 | 10.2 | 0.5 |

TABLE 3
TAG profiles of palm oil. (M, myristic acid; O, oleic acid;
P, palmitic acid; L, linoleic acid; S, stearic acid)

| Unsaturated TAG | OLL | PLL | OLO | POL | PLP | OOO | POO | POP | SOO | POS |
|---|---|---|---|---|---|---|---|---|---|---|
| Content (%) | 0.4 | 1.2 | 1.5 | 8.9 | 9.2 | 3.9 | 23.2 | 30.2 | 2.9 | 6.7 |

| Saturated TAG | PPM | PPP | PPS | Others |
|---|---|---|---|---|
| Content (%) | 0.2 | 6.7 | 1.1 | 3.8 |

### Analytical Methods for PMTAG and Fractions of PMTAG

The solid and liquid fractions of PMTAG were analyzed using different techniques. These techniques can be broken down into: (i) chemistry characterization techniques, including iodine value, acid value, nuclear magnetic resonance (NMR), and high pressure liquid chromatography (HPLC), including fast and slow methods of the HPLC; and (ii) physical characterization methods, including thermogravimetric analysis (TGA), differential scanning calorimetry (DSC), rheology, and solid fat content (SFC).

### Chemistry Characterization Techniques

Iodine and acid values of the solid and liquid fractions of PMTAG were determined according to ASTM D5554-95 and ASTM D4662-03, respectively.

¹H-NMR spectra were recorded on a Varian Unity-INOVA at 499.695 MHz. ¹H chemical shifts are internally referenced to CDCl₃ (7.26 ppm) for spectra recorded in CDCl₃. All spectra were obtained using an 8.6 is pulse with 4 transients collected in 16 202 points. Datasets were zero-filled to 64 000 points, and a line broadening of 0.4 Hz was applied prior to Fourier transforming the sets. The spectra were processed using ACD Labs NMR Processor, version 12.01.

HPLC analysis was performed on a Waters Alliance (Milford, Mass.) e2695 HPLC system fitted with a Waters ELSD 2424 evaporative light scattering detector. The HPLC system was equipped with an inline degasser, a pump, and an auto-sampler. The ELSD nitrogen flow was set at 25 psi with nebulization and drifting tube maintained at 12° C. and 55° C., respectively. Gain was set at 500. All solvents were HPLC grade and obtained from VWR International, Mississauga, ON. Waters Empower Version 2 software was used for data collection and data analysis. Purity of eluted samples was determined using the relative peak area. The analysis was performed on a C18 column (150 mm×4.6 mm, 5.0 μm, X-Bridge column, Waters Corporation, MA) maintained at 30° C. by column oven (Waters Alliance). The mobile phase was chloroform: acetonitrile (20:80)v run for 80 min at a flow rate of 0.5 ml/min. 5 mg/ml (w/v) solution of crude sample in chloroform was filtered through single step filter vial (Thomson Instrument Company, 35540, CA) and 5 μL of sample was passed through the C18 column by reversed-phase in isocratic mode.

### Physical Characterization Techniques

TGA was carried out on a TGA Q500 (TA Instruments, DE, USA) equipped with a TGA heat exchanger (P/N 953160.901). Approximately 8.0-15.0 mg of sample was loaded in the open TGA platinum pan. The sample was heated from 25 to 600° C. under dry nitrogen at a constant rate of 10° C./min.

DSC measurements were run on a Q200 model (TA Instruments, New Castle, Del.) under a nitrogen flow of 50 mL/min. TAG samples between 3.5 and 6.5 (±0.1) mg were run in hermetically sealed aluminum DSC pans. Crystallization and melting behavior was investigated using standard DSC. The sample was equilibrated at 90° C. for 10 min to erase thermal memory, and then cooled at a constant rate of 5.0° C./min to −90° C. where it was held isothermally for 5 min, and subsequently reheated at a constant rate of 5.0° C./min to 90° C. The “TA Universal Analysis” software was used to analyze the DSC thermograms and extract the peak characteristics. Characteristics of non-resolved peaks were obtained using the first and second derivatives of the differential heat flow.

SFC measurements were performed on a Bruker Minispec mq 20 pNMR spectrometer (Milton, ON, Canada) equipped with a combined high and low temperature probe supplied with N2. The temperature was controlled with Bruker's BVT3000 temperature controller with an accuracy of ±0.1° C. The temperature was calibrated with commercial canola oil using a type K probe (TRP-K, Omega, Stamford, Conn.) immersed in the oil and an external data logger (Oakton, Eutech Instruments, Singapore). Approximately 0.57±0.05 ml of fully melted sample was quickly pipetted into the bottom portion of the NMR tube. The thermal protocol used in the DSC were also used in the NMR. Bruker's minispec V2.58 Rev. 12 and minispec plus V1.1 Rev. 05 software were used to collect SFC data as a function of time and temperature. The SFC values are reported as the ratio of the intensity of the NMR signal of the solid part to the total detected NMR signal in percent (labelled as SFC %).

A temperature-controlled Rheometer (AR2000ex, TA Instruments, DE, USA) was used to measure the viscosity and flow property of MTAG using a 40 mm 2° steel geometry. Temperature control was achieved by a Peltier attachment with an accuracy of 0.1° C. Shear Stress was measured at each temperature by varying the shear rate from 1 to 1200 s⁻¹. Measurements were taken at 10° C. intervals from high temperature (100° C.) to 10° C. below the DSC onset of crystallization temperature of each sample. Viscosities of samples were measured from each sample's melting point up to 110° C. at constant temperature rate (1.0 and 3.0° C./min) with constant shear rate (200 s⁻¹). Data points were collected at intervals of 1° C. The viscosity obtained in this manner was in very good agreement with the measured viscosity using the shear rate/share stress. The shear rate range was optimized for torque (lowest possible is 10 μNm) and velocity (maximum suggested of 40 rad/s).

The shear rate—shear stress curves were fitted with the Herschel-Bulkley equation (Eq 1), a model commonly used to describe the general behavior of materials characterized by a yield stress.

τ=τ₀+K\dot{γ}ⁿ Eq. 1

where \dot{γ} denotes the shear stress, τ₀ is the yield stress below which there is no flow, K is the consistency index and n is the power index, ₙ depends on constitutive properties of the material. For Newtonian fluids n=1, for shear thickening fluids n>1 and for shear thinning fluids n<1.

### Fractionation of MTAG of Palm Oil

The fractionation of PMTAG was achieved based on its crystallization and melting behaviors. Dry and solvent aided crystallization procedures were used to separate the PMTAG into a high and low melting temperature fractions, referred to as the solid and liquid fractions, respectively. Dichloromethane (DCM) was used in the so-called solvent fractionation. The details of the procedures are presented in following sections. The liquid fractions as well as solid fractions of the PMTAG were epoxidized then hydroxylated and/or hydrogenated to make polyols. The polyols obtained from the liquid fractions were used to make rigid and flexible foams.

### Potential Composition of Liquid and Solid Fractions of MTAG of Palm Oil

The natural oil composition, and in particular, the palm oil composition, was described previously in commonly assigned U.S. Provisional Patent Application Ser. No. 61/971,475, and the TAG profiles of palm oil were also described previously in the literature. The possible structures of the PMTAG fractions based on the compositional analysis of PMTAG itself are presented in Scheme 4. These contain fatty acids with terminal double bonds, internal double bonds with n=2 or 8, as well as saturated fatty acids with m=11 to 20.

The TAGs which can potentially compose PMTAG and its fractions based on palm oil composition and the possible products of cross-metathesis of palm oil are listed in Table 4a. The corresponding structures are listed in Table 4b.

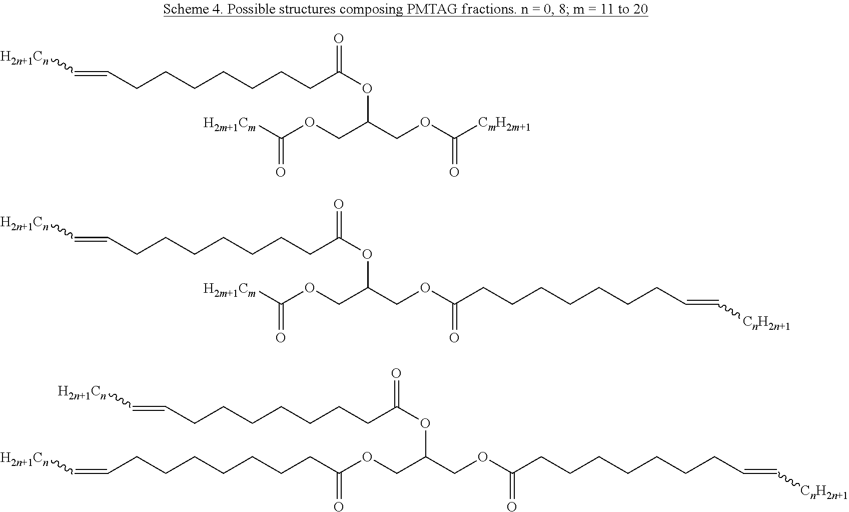

TABLE 4a
Potential TAG composition in PMTAG fractions. D: 9-decenoic
acid; Dd: 9-dodecenioc acid; M, myristic acid; O, oleic acid;
P, palmitic acid; L, linoleic acid; S, stearic acid. There
are both trans- and cis- double bonds in the TAG.

| TAGs in Palm oil | Potential TAG composition of PMTAG |
|---|---|
| OLL, OLO, OOO | ODD, DDD, DDDd, DDdDd, OLL, OLD, OLDd, |
| | DdDdDd, and their isomers |
| PLL | PLL, PDD, PLD, PDDd, PLDd, PDdDd and |
| | their isomers |
| POL, POO | POL, POO, PDD, POD, PDDd, PODd, PDdDd and |
| | their isomers |
| SOO | POO, PDD, POD, PDDd, PODd, PDdDd and |
| | their isomers |
| PLP, | PLP, PDP, PDdP |
| POP | POP, PDP, PDdP |
| POS | POS, PDS, PDdS |
| PPM, PPP, PPS | PPM, PPP, PPS |

TABLE 4b
Structures of potential TAG composition in PMTAG and PMTAG fractions.

| Compounds | Structures |
|---|---|
| OLL | 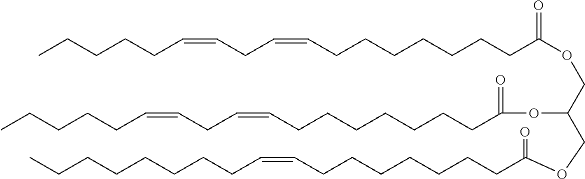 [SMILES: CCCCC/C=C\C/C=C\CCCCCCCC(=O)OCC(COC(=O)CCCCCCC/C=C\CCCCCCCC)OC(=O)CCCCCCC/C=C\C/C=C\CCCCC] |
| OLO | 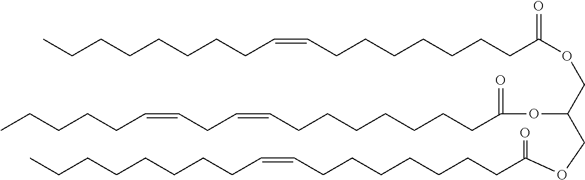 [SMILES: CCCCC/C=C\C/C=C\CCCCCCCC(=O)OC(COC(=O)CCCCCCC/C=C\CCCCCCCC)COC(=O)CCCCCCC/C=C\CCCCCCCC] |
| OOO | 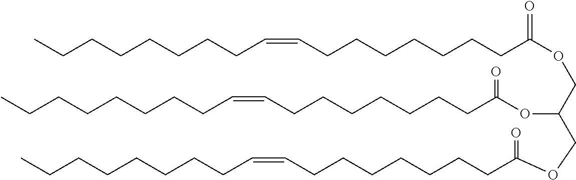 [SMILES: CCCCCCCC/C=C\CCCCCCCC(=O)OCC(COC(=O)CCCCCCC/C=C\CCCCCCCC)OC(=O)CCCCCCC/C=C\CCCCCCCC] |
| ODD | 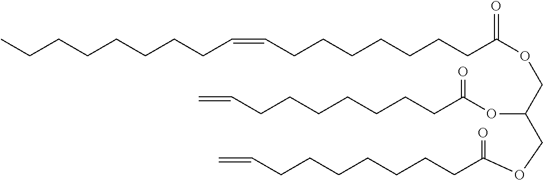 [SMILES: C=CCCCCCCCC(=O)OCC(COC(=O)CCCCCCC/C=C\CCCCCCCC)OC(=O)CCCCCCCC=C] |
| DDD | 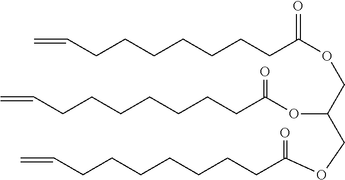 [SMILES: C=CCCCCCCCC(=O)OCC(COC(=O)CCCCCCCC=C)OC(=O)CCCCCCCC=C] |
| DDDd | 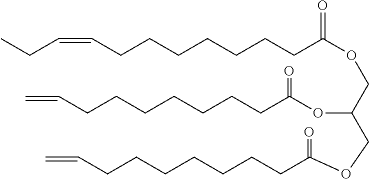 [SMILES: C=CCCCCCCCC(=O)OCC(COC(=O)CCCCCCC/C=C\CC)OC(=O)CCCCCCCC=C] |
| DDdDd | 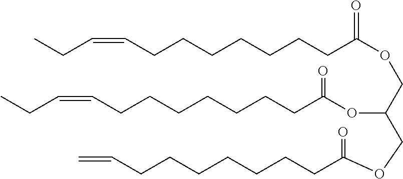 [SMILES: C=CCCCCCCCC(=O)OCC(COC(=O)CCCCCCC/C=C\CC)OC(=O)CCCCCCC/C=C\CC] |
| OLD | 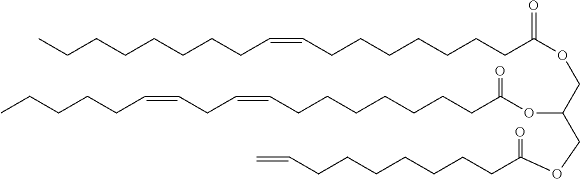 [SMILES: C=CCCCCCCCC(=O)OCC(COC(=O)CCCCCCC/C=C\CCCCCCCC)OC(=O)CCCCCCC/C=C\C/C=C\CCCCC] |
| OLDd | 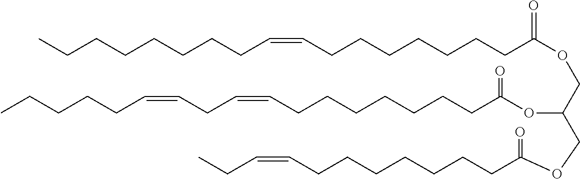 [SMILES: CC/C=C\CCCCCCCC(=O)OCC(COC(=O)CCCCCCC/C=C\CCCCCCCC)OC(=O)CCCCCCC/C=C\C/C=C\CCCCC] |
| OOD | 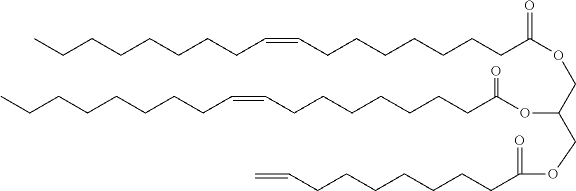 [SMILES: C=CCCCCCCCC(=O)OCC(COC(=O)CCCCCCC/C=C\CCCCCCCC)OC(=O)CCCCCCC/C=C\CCCCCCCC] |
| ODD | 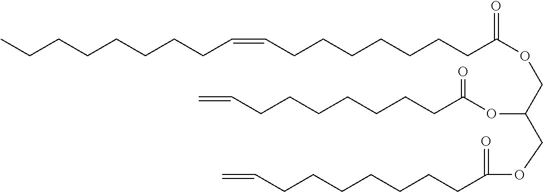 [SMILES: C=CCCCCCCCC(=O)OCC(COC(=O)CCCCCCC/C=C\CCCCCCCC)OC(=O)CCCCCCCC=C] |
| ODDd | 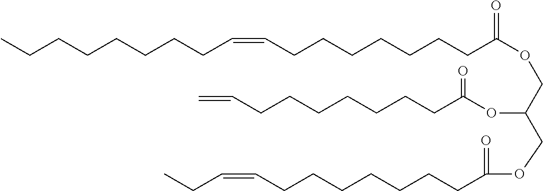 [SMILES: C=CCCCCCCCC(=O)OC(COC(=O)CCCCCCC/C=C\CC)COC(=O)CCCCCCC/C=C\CCCCCCCC] |
| ODdDd | 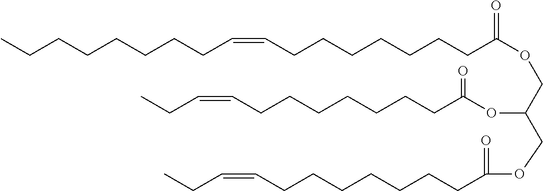 [SMILES: CC/C=C\CCCCCCCC(=O)OCC(COC(=O)CCCCCCC/C=C\CCCCCCCC)OC(=O)CCCCCCC/C=C\CC] |
| LDD | 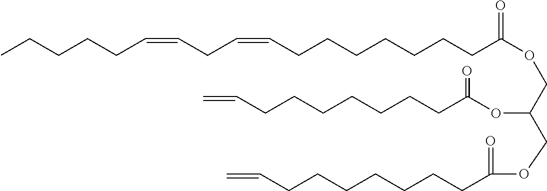 [SMILES: C=CCCCCCCCC(=O)OCC(COC(=O)CCCCCCC/C=C\C/C=C\CCCCC)OC(=O)CCCCCCCC=C] |
| LDDd | 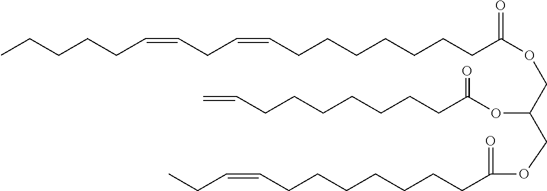 [SMILES: C=CCCCCCCCC(=O)OC(COC(=O)CCCCCCC/C=C\CC)COC(=O)CCCCCCC/C=C\C/C=C\CCCCC] |
| LDdDd | 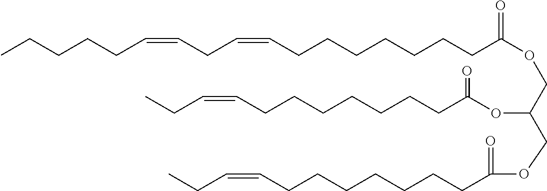 [SMILES: CC/C=C\CCCCCCCC(=O)OCC(COC(=O)CCCCCCC/C=C\C/C=C\CCCCC)OC(=O)CCCCCCC/C=C\CC] |
| DdDdDd | 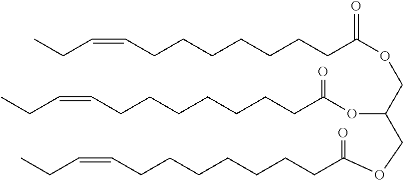 [SMILES: CC/C=C\CCCCCCCC(=O)OCC(COC(=O)CCCCCCC/C=C\CC)OC(=O)CCCCCCC/C=C\CC] |
| PLL | 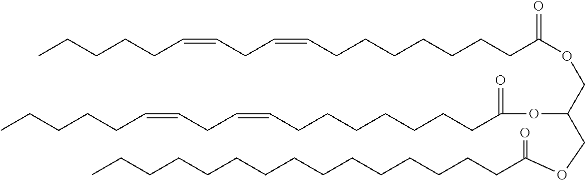 [SMILES: CCCCC/C=C\C/C=C\CCCCCCCC(=O)OCC(COC(=O)CCCCCCCCCCCCCCC)OC(=O)CCCCCCC/C=C\C/C=C\CCCCC] |
| PDD | 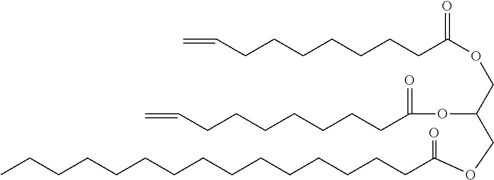 [SMILES: C=CCCCCCCCC(=O)OCC(COC(=O)CCCCCCCCCCCCCCC)OC(=O)CCCCCCCC=C] |
| PLD | 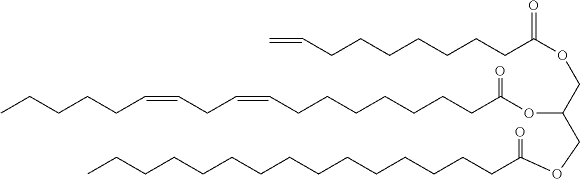 [SMILES: C=CCCCCCCCC(=O)OCC(COC(=O)CCCCCCCCCCCCCCC)OC(=O)CCCCCCC/C=C\C/C=C\CCCCC] |
| PDDd | 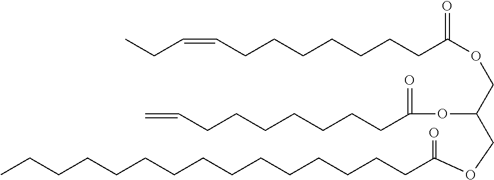 [SMILES: C=CCCCCCCCC(=O)OC(COC(=O)CCCCCCC/C=C\CC)COC(=O)CCCCCCCCCCCCCCC] |
| PLDd | 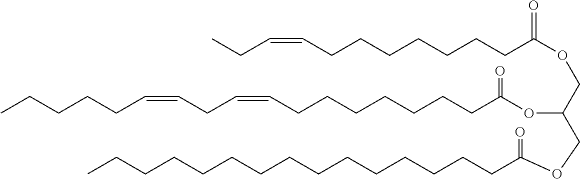 [SMILES: CC/C=C\CCCCCCCC(=O)OCC(COC(=O)CCCCCCCCCCCCCCC)OC(=O)CCCCCCC/C=C\C/C=C\CCCCC] |
| PDdDd | 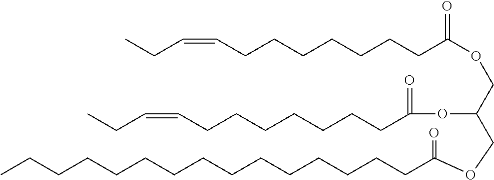 [SMILES: CC/C=C\CCCCCCCC(=O)OCC(COC(=O)CCCCCCCCCCCCCCC)OC(=O)CCCCCCC/C=C\CC] |
| POL | 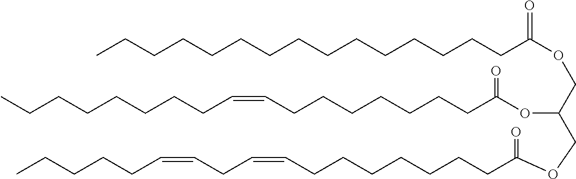 [SMILES: CCCCC/C=C\C/C=C\CCCCCCCC(=O)OCC(COC(=O)CCCCCCCCCCCCCCC)OC(=O)CCCCCCC/C=C\CCCCCCCC] |
| POO | 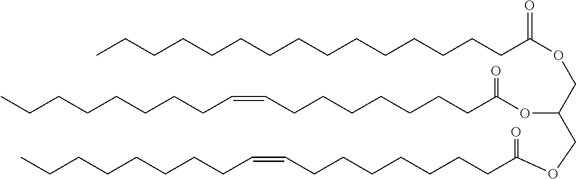 [SMILES: CCCCCCCC/C=C\CCCCCCCC(=O)OCC(COC(=O)CCCCCCCCCCCCCCC)OC(=O)CCCCCCC/C=C\CCCCCCCC] |
| POD | 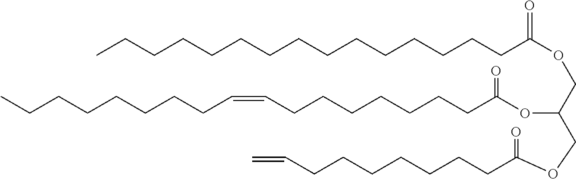 [SMILES: C=CCCCCCCCC(=O)OCC(COC(=O)CCCCCCCCCCCCCCC)OC(=O)CCCCCCC/C=C\CCCCCCCC] |
| PODd | 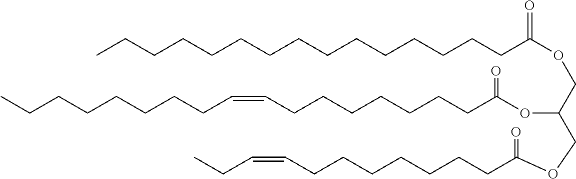 [SMILES: CC/C=C\CCCCCCCC(=O)OCC(COC(=O)CCCCCCCCCCCCCCC)OC(=O)CCCCCCC/C=C\CCCCCCCC] |
| SOO | 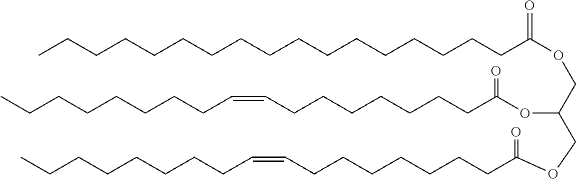 [SMILES: CCCCCCCC/C=C\CCCCCCCC(=O)OCC(COC(=O)CCCCCCCCCCCCCCCCC)OC(=O)CCCCCCC/C=C\CCCCCCCC] |
| SDD | 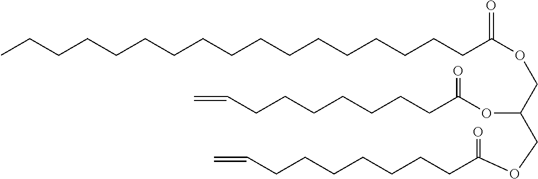 [SMILES: C=CCCCCCCCC(=O)OCC(COC(=O)CCCCCCCCCCCCCCCCC)OC(=O)CCCCCCCC=C] |
| SOD | 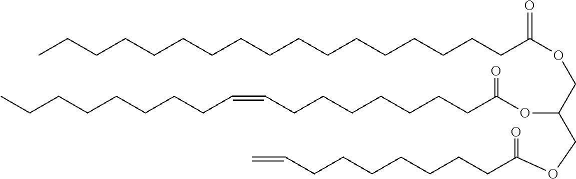 [SMILES: C=CCCCCCCCC(=O)OCC(COC(=O)CCCCCCCCCCCCCCCCC)OC(=O)CCCCCCC/C=C\CCCCCCCC] |
| SDDd | 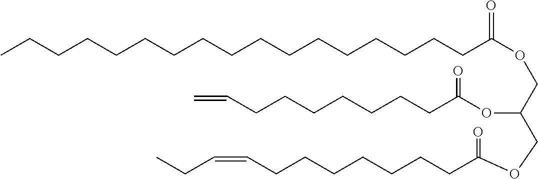 [SMILES: C=CCCCCCCCC(=O)OC(COC(=O)CCCCCCC/C=C\CC)COC(=O)CCCCCCCCCCCCCCCCC] |
| SODd | 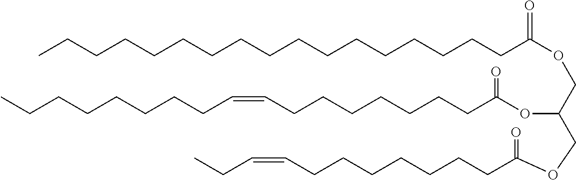 [SMILES: CC/C=C\CCCCCCCC(=O)OCC(COC(=O)CCCCCCCCCCCCCCCCC)OC(=O)CCCCCCC/C=C\CCCCCCCC] |
| SDdDd | 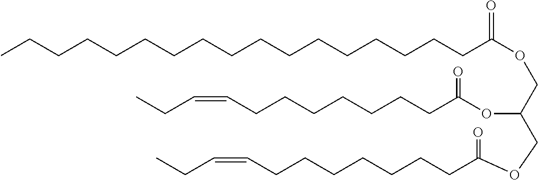 [SMILES: CC/C=C\CCCCCCCC(=O)OCC(COC(=O)CCCCCCCCCCCCCCCCC)OC(=O)CCCCCCC/C=C\CC] |
| PLP | 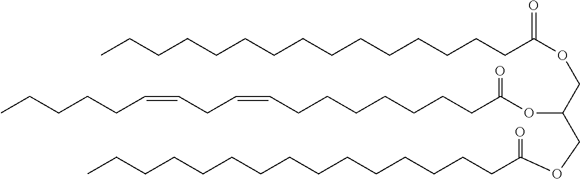 [SMILES: CCCCC/C=C\C/C=C\CCCCCCCC(=O)OC(COC(=O)CCCCCCCCCCCCCCC)COC(=O)CCCCCCCCCCCCCCC] |
| PDP | 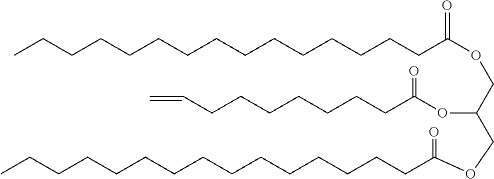 [SMILES: C=CCCCCCCCC(=O)OC(COC(=O)CCCCCCCCCCCCCCC)COC(=O)CCCCCCCCCCCCCCC] |
| PDdP | 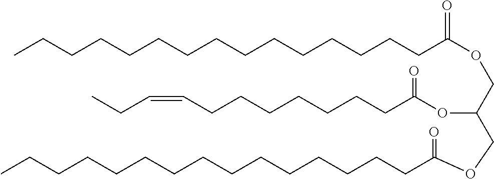 [SMILES: CC/C=C\CCCCCCCC(=O)OC(COC(=O)CCCCCCCCCCCCCCC)COC(=O)CCCCCCCCCCCCCCC] |
| POP | 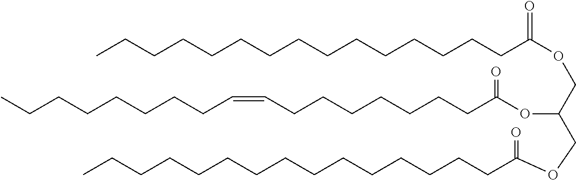 [SMILES: CCCCCCCC/C=C\CCCCCCCC(=O)OC(COC(=O)CCCCCCCCCCCCCCC)COC(=O)CCCCCCCCCCCCCCC] |
| POS | 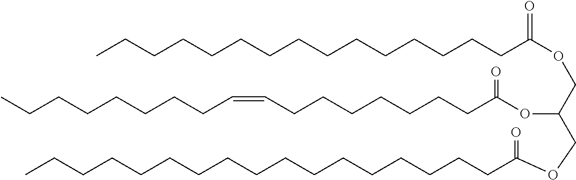 [SMILES: CCCCCCCC/C=C\CCCCCCCC(=O)OC(COC(=O)CCCCCCCCCCCCCCC)COC(=O)CCCCCCCCCCCCCCCCC] |
| PDS | 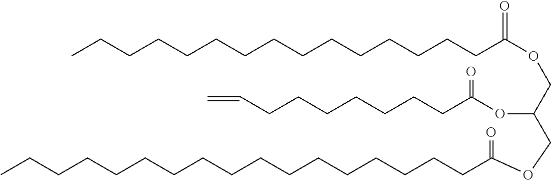 [SMILES: C=CCCCCCCCC(=O)OC(COC(=O)CCCCCCCCCCCCCCC)COC(=O)CCCCCCCCCCCCCCCCC] |
| PDdS | 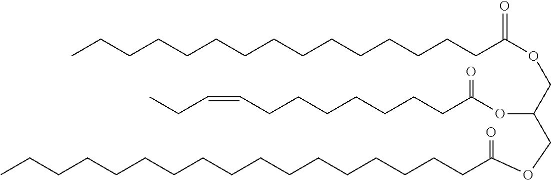 [SMILES: CC/C=C\CCCCCCCC(=O)OC(COC(=O)CCCCCCCCCCCCCCC)COC(=O)CCCCCCCCCCCCCCCCC] |
| PPM | 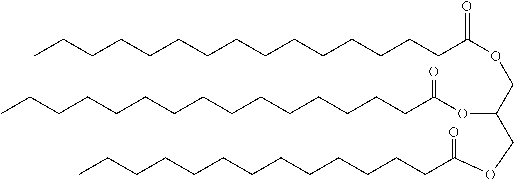 [SMILES: CCCCCCCCCCCCCCCC(=O)OCC(COC(=O)CCCCCCCCCCCCC)OC(=O)CCCCCCCCCCCCCCC] |
| PPP | 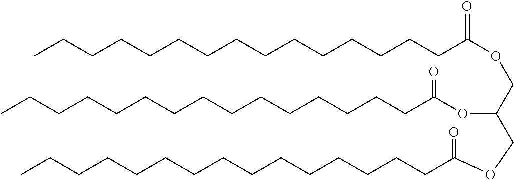 [SMILES: CCCCCCCCCCCCCCCC(=O)OCC(COC(=O)CCCCCCCCCCCCCCC)OC(=O)CCCCCCCCCCCCCCC] |
| PPS | 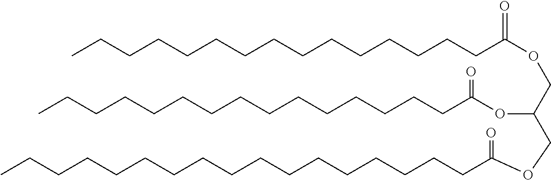 [SMILES: CCCCCCCCCCCCCCCCCC(=O)OCC(COC(=O)CCCCCCCCCCCCCCC)OC(=O)CCCCCCCCCCCCCCC] |

### Crystallization and Melting Behavior of Palm Oil MTAG

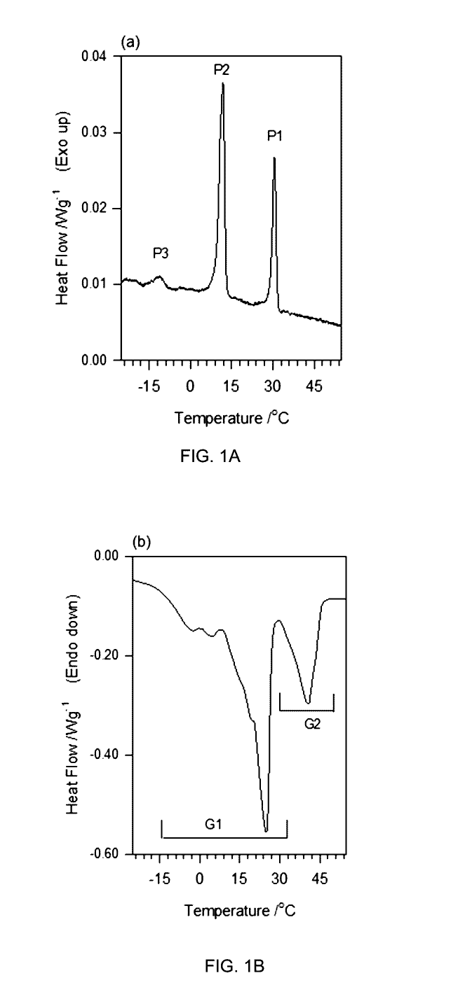

The fractionation by crystallization of PMTAG can be understood in light of its thermal transition behavior. The DSC thermogram obtained on cooling PMTAG at 0.1° C./min and the thermogram obtained by subsequent heating at 5° C./min are presented in FIGS. 1A and 1B, respectively.

As can be seen in FIGS. 1A and 1B, PMTAG cooling thermogram presented three exotherms and its heating thermogram presented two relatively well-separated groups of endotherms (G1 below 30° C. and G2 above 30° C. in FIG. 1B) indicating separate high and low temperature fractions of the MTAG. Similarly to its palm oil starting material, the thermal events that appeared above room temperature (exotherm at ˜32° C., P1 in FIG. 1A, and melting counterpart G2 in FIG. 1B) are associated with a stearin-like fraction of the MTAG and the thermal events that appeared below room temperature and at sub-zero temperatures (exotherms at ˜12 and −11° C., P2 and P3, respectively, in FIG. 1A, and melting counterpart G1 in FIG. 1B) to its olein-like fraction. This indicates that with careful processing, it is possible to separate PMTAG into two fractions: a portion that is rich in cis-/short chains (olein-like portion) that would remain liquid at ambient (so-called liquid fraction, LF), and a portion that is rich in trans-/long chains (stearin-like fraction) that would be solid at ambient (so-called solid fraction, SF).

PMTAG has been separated into a solid and liquid fractions using three methods: I. Dry fractionation by slow cooling at a fixed rate followed by isothermal crystallization, II. Dry fractionation by quiescent cooling and isothermal crystallization, and III. Solvent aided crystallization. In the following, the liquid and solid fractions of PMTAG are labeled LF-PMTAG and SF-PMTAG, respectively. The fractions obtained by dry fractionation—rates method—are specified with the acronym D1 and labeled LF(D1)-MTAG, and SF(D1)-MTAG, respectively, those obtained with dry fractionation—quiescent method—are specified with the acronym D2 and labeled LF(D2)-MTAG and SF(D2)-MTAG, respectively, and those obtained with solvent are specified with the acronym S and labeled LF(S)-MTAG and SF(S)-MTAG, respectively. The detailed nomenclature used in the document is presented in Table 1.

### Liquid and Solid Fractionations of PMTAG

### Fractionation of PMTAG by Dry Crystallization—Rates Method (D1)

In the dry fractionation procedure—Rates Method (D1), the sample was cooled very slowly from the melt at a prescribed rate down to a temperature (T_{C}) at which it was crystallized isothermally for a fixed period of time (t_{C}). The crystallized material (solid fraction) was then filtered from the liquid phase (liquid fraction). T_{C} and t_{C} were chosen to promote the crystallization of the stearin portion of PMTAG only. In order to control the fractionation and maximize yield, four sets of experiments were conducted (F1 to F4 in Table 5). The experiments combine two cooling rates (0.05 or 0.035° C./min) with a T_{C} chosen within the span of the PMTAG stearin crystallization.

Practically, ˜200 to 260 g of melted PMTAG in a round bottom flask was placed in a temperature controlled water bath (Julabo FP50-ME, Julabo USA Inc., Vista, Calif.) already set at 90° C. The sample was cooled at the prescribed rate and crystallized under vigorous stirring (500 rpm). The solid fraction was filtered from the liquid fraction with filter paper (Fisherbrand™, P5) and the help of a vacuum pump (BUCHI V-700, Switzerland). The details of the different experiments and the results of the fractionations are listed in Table 5.

TABLE 5
PMTAG dry fractionation data. LF: Liquid fraction of PMTAG; SF:
Solid fraction of PMTAG; T_{C}(° C.): crystallization temperature; and
t_{C} (h): crystallization time. Yield of liquid fraction (%)

| | Mass | Cooling Rate | T_{C} | t_{C} | LF | SF | Yield |
|---|---|---|---|---|---|---|---|
| Experiment | (g) | (° C./min) | (° C.) | (h) | (g) | (g) | (%) |
| F1 | 200 | 0.050 | 35.0 | 7.0 | 55 | 145 | 27.5 |
| F2 | 250 | 0.035 | 39.5 | 9.0 | 112 | 133 | 44.8 |
| F3 | 244 | 0.035 | 35.0 | 6.5 | 89 | 155 | 36.5 |
| F4 | 258 | 0.035 | 29.0 | 11.0 | 55 | 203 | 21.5 |

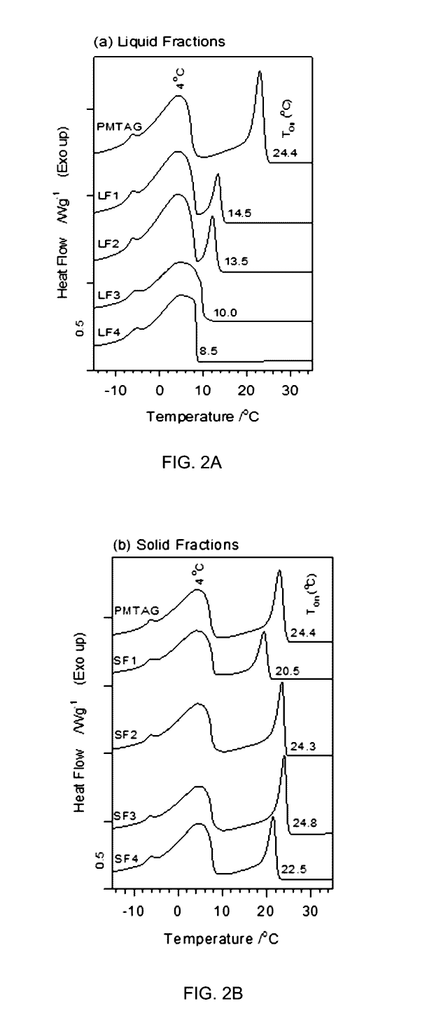

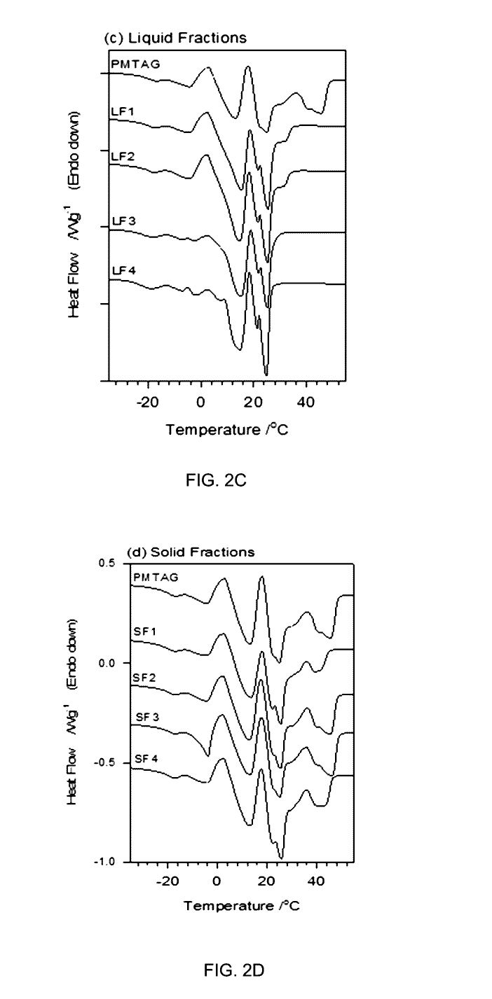

The DSC cooling thermograms (5.0° C./min) of the liquid and solid fractions obtained by dry fractionation of MTAG of palm oil are presented in FIGS. 2A and 2B, respectively, and the thermograms obtained by subsequent heating (5° C./min) are presented in FIGS. 2C and 2D, respectively.

As can be seen in FIG. 2A, the procedure was effective. For example, in experiments F3 and F4, only the exotherms associated with the olein portion of PMTAG was presented in the thermograms of the liquid fraction (LF3 and LF4 curves in FIG. 2A). The yield of liquid fraction, however, was relatively small (˜37 and 22% wt in F3 and F4, respectively). In experiments F1 and F2, the cooling thermograms of the liquid fractions (LF1 and LF2 curves in FIG. 2A), presented exotherms of both PMTAG stearin and PMTAG olein. However, their onset temperatures of crystallization were much lower (14.5 and 13.5° C., respectively) compared to PMTAG (22.4° C.), indicating that the liquid fraction retained some of the lower melting components of PMTAG stearin. In all the experiments, the cooling thermograms of the solid fraction displayed both the high and low temperature exotherms, indicating that a significant part of the PMTAG olein portion was retained in the solid fraction.

### Standard Dry Fractionation Procedure D1

The dry crystallization procedure outlined for F2 which achieved the highest yield of liquid fraction (˜45%) was used to produce the standard solid and liquid fractions of the MTAG of palm oil.

### Fractionation of PMTAG by Dry Crystallization 2—Quiescent Method

In the second dry fractionation procedure (D2), the sample was brought from the melt (T_{M}) directly to a temperature (T_{C}) at which it was crystallized isothermally for a period of time (t_{C}). The crystallized material (solid fraction) was then filtered from the liquid phase (liquid fraction). Four sets of experiments, in which T_{M}, T_{C} and t_{C} were chosen so to promote the crystallization of the PMTAG stearin and achieve high yield for the liquid fraction, were conducted (F1 to F4 in Table 6). The experiments combine melting temperatures (60, 55 and 50° C. in Table 6) with a T_{C} chosen within the span of the PMTAG stearin crystallization.

Practically, ˜60 g of melted PMTAG in 100-ml beaker was placed in a temperature controlled water bath (Julabo FP50-ME, Julabo USA Inc., Vista, Calif.) already set at T_{M}. The sample was placed directly in an incubator already set at T_{C} and crystallized isothermally during T_{C}. The solid fraction was filtered from the liquid fraction with filter paper (Fisherbrand™, P5) under vacuum (300 torr) at the crystallization temperature. The yield of liquid fraction was higher than 62% wt in all experiments. The details of the different experiments and the results of the fractionations are listed in Table 6.

TABLE 6
PMTAG fractionation data (dry - quiescent method, D2). LF: Liquid
fraction of PMTAG; SF: Solid fraction of PMTAG; T_{M} (° C.): melting
temperature, T_{C}(° C.): isothermal crystallization temperature, and
Tₒₙ (° C.): DSC onset of crystallization temperature; and t_{C}
(h): crystallization time. Yield of liquid fraction (%)

| | Mass | T_{M} | T_{C} | t_{C} | Tₒₙ | Yield |
|---|---|---|---|---|---|---|
| Experiment | (g) | (° C.) | (° C.) | (h) | (° C.) | (%) |
| F1 | 62 | 60 | 35.0 | 22 | 18.2 | 65.3 |
| F2 | 62 | 60 | 35.0 | 46 | 17.7 | 62.9 |
| F3 | 62 | 55 | 33.0 | 24 | 17.9 | 72.5 |
| F4 | 62 | 50 | 31.5 | 24 | 13.8 | 64.5 |

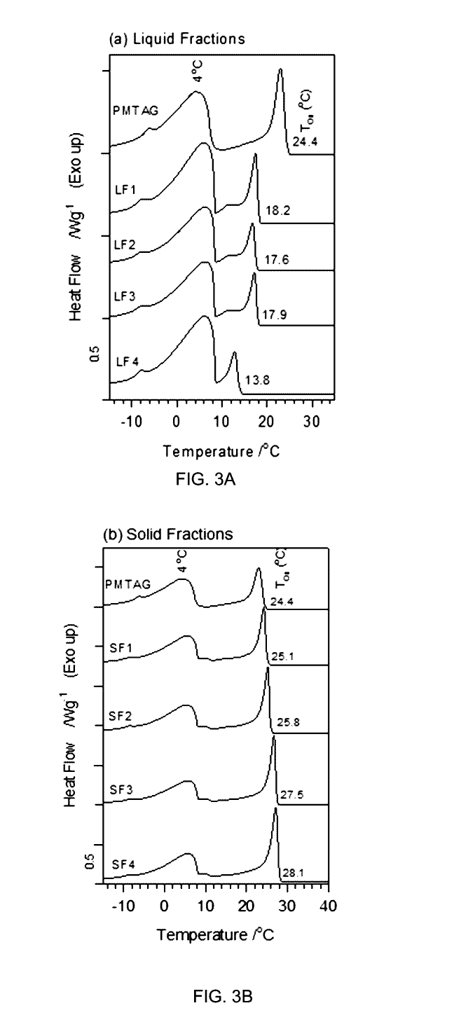

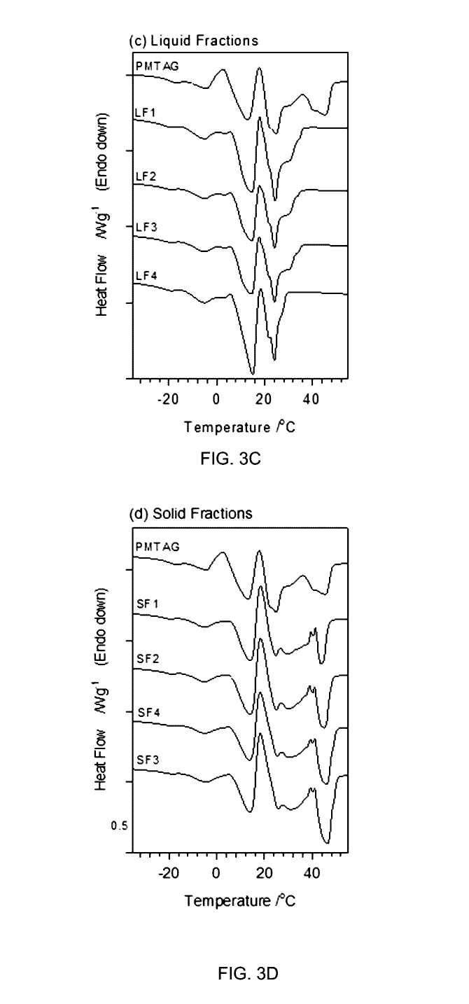

The DSC cooling thermograms (5.0° C./min) of the liquid and solid fractions obtained by quiescent fractionation of PMTAG are presented in FIGS. 3A and 3B, respectively, and the thermograms obtained by subsequent heating (5° C./min) are presented in FIGS. 3C and 3D, respectively. In all experiments, the cooling thermograms of the liquid fractions (LF1 to LF4 curves in FIG. 3A), presented the high and low temperature exotherms of the PMTAG, indicating the presence of both stearin and olein portions of the PMTAG. However, the onset of crystallization as well as the enthalpy of the first exotherm, which is associated with the stearin portion of PMTAG, were decreased. This indicates that the liquid fraction was depleted from the stearin portion noticeably, and that the components crystallizing at the highest temperatures were filtered out. In all the experiments, the cooling thermograms of the solid fraction displayed both the high and low temperature exotherms, indicating that a significant part of the PMTAG olein was retained in the solid fraction.

### Standard Dry Fractionation Procedure D2

The dry crystallization procedure D2 outlined for F4 which has achieved the lowest T_{On} (13.8° C.) was used to produce the standard solid and liquid fractions of the MTAG of palm oil.

### Solvent Fractionation of MTAG of Palm Oil

In the solvent fractionation, melted PMTAG was mixed under gentle stirring with dichloromethane (DCM) in a 20-L jacketed reactor (Heb Biotechnology Co., Ltd, Xi'an, China). The reactor was connected to a temperature controlled circulator (Hack Phonex II P1 Circulator, Thermo Electron, Karlsruhe, Germany). The MTAG was dissolved in DCM at T_{disol} then brought to a crystallization temperature T_{C} that allows for the stearin fraction of the MTAG to crystallize isothermally and eventually sediment. The solvent type (DCM) and PMTAG: DCM ratio were chosen so that the products of the fractionation can be used in the epoxidation step of the synthesis of the polyols without further separation steps.

### Standard Solvent Fractionation Procedure (S)

The standard solid fraction and liquid fractions of the MTAG of palm oil was produced as follow: ˜5 kg (3.8 L) of DCM was added to 5 kg of melted PMTAG (PMTAG to DCM ratio of 1:1 (wt/wt)) in the reactor already set at 37° C. The MTAG was fully dissolved at this temperature. The mixture was then left to cool down to 2° C. under stirring. The stirring was turned off and the mixture was left to crystallize for 24 h at this temperature. The crystallized material (so-called solid fraction or SF) was then filtered from the liquid (so-called liquid fraction or LF) with filter paper (Fisherbrand™, P8, 15 cm). The two fractions were separated easily and very effectively with vacuum (300 Torr). The solvent fractionation procedure achieved a high yield of liquid fraction of ˜70%. The results of the fractionation are listed in Table 7. Note that the solid fraction was dried completely and that 1 L of DCM was added to the liquid fraction and used to make a polyol.

TABLE 7
PMTAG solvent fractionation data. LF(S)-PMTAG and SF(S)-
PMTAG: Liquid and solid fractions of PMTAG, respectively. T_{disol}
(° C.): dissolution temperature; T_{C}(° C.): crystallization
temperature; t_{C} (h): crystallization time

| PMTAG | DCM | | | | | | |
|---|---|---|---|---|---|---|---|
| Mass | Volume | T_{disol} | T_{C} | t_{C} | LF | SF | Yield |
| (kg) | (L) | (° C.) | (° C.) | (h) | (kg) | (kg) | (%) |
| 5.0 | 7.2 | 37 | 2 | 24 | 3.8 | 1.6 | 70.3 |

### Standard Fractionation Procedures

The iodine and acid values of the standard solid and liquid fractions of PMTAG obtained with dry fractionation (D1 and D2), and solvent fractionation (S) are listed in Table 8.

TABLE 8
Iodine and acid values of the standard solid and liquid
fractions of PMTAG obtained with dry fractionation (methods
D1 and D2) and solvent aided fractionation (S)

| Liquid Fractions | | | Solid Fractions | | |
|---|---|---|---|---|---|
| | Iodine | Acid value | | Iodine | Acid value |
| | Value | (mg KOH/g) | | Value | (mg KOH/g) |
| LF(D1)- | 60.4 | 0.77 | SF(D1)- | 35.5 | 0.47 |
| PMTAG | | | PMTAG | | |
| LF(D2)- | 60 | 0.81 | SF(D2)- | 35 | 0.57 |
| PMTAG | | | PMTAG | | |
| LF(S)- | 59.6 | 0.75 | SF(S)- | 35.3 | 0.48 |
| PMTAG | | | PMTAG | | |

### Compositional Analysis of the PMTAG Fractions

### Fatty Acid and TAG Profiles of PMTAG Fractions

The fatty acid profiles of the liquid and solid fractions of PMTAG (LF-PMTAG and SF-PMTAG, respectively) was determined using ¹H-NMR data. TAG profiles of SF-PMTAG and LF-PMTAG were determined with HPLC. Three pure TAGs, namely 3-(stearoyloxy) propane-1,2-diyl bis(dec-9-enoate), or DSS, 3-(dec-9-enoyloxy) propane-1,2-diyl distearate or DDS, and 1, 2, 3-triyl tris (dec-9-enoate) or DDD were synthesized and used as standards to help in the determination of the TAG profile of the MTAG.

### ¹H-NMR of PMTAG Fractions

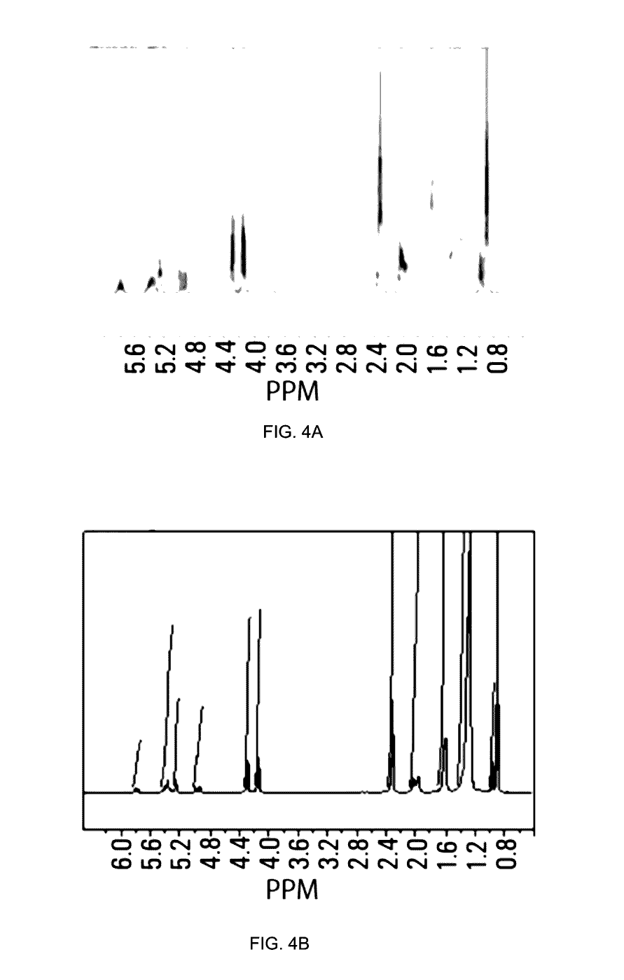

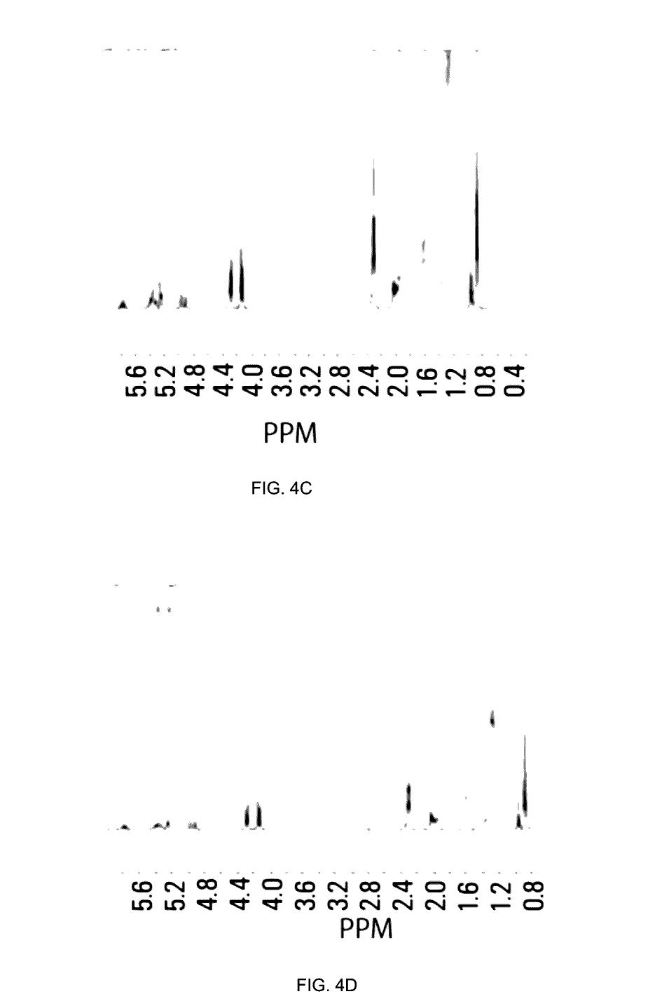

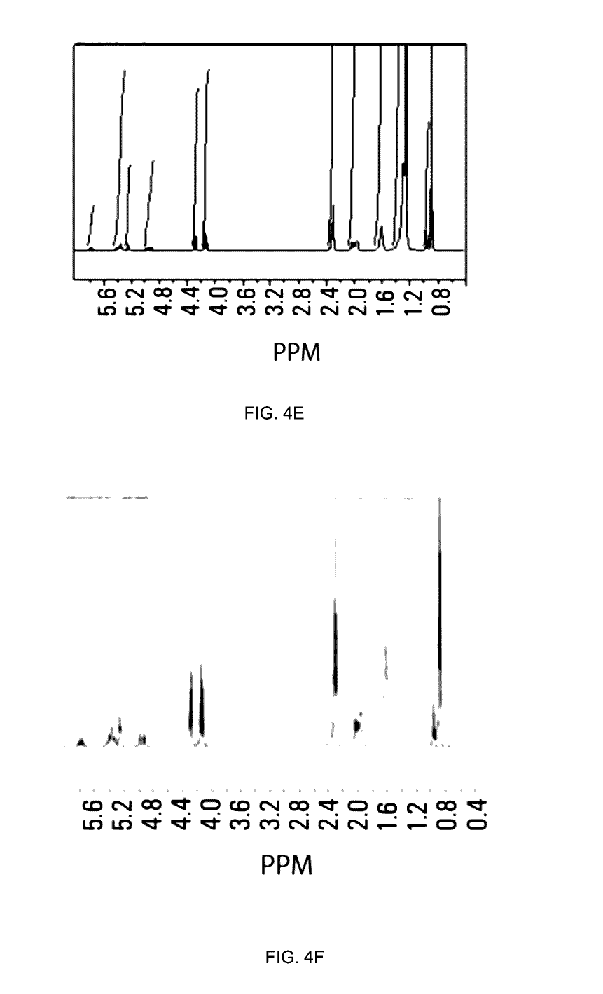

¹H-NMR spectra of SF-PMTAG are shown in FIGS. 4A-4C and those of LF-PMTAG in FIGS. 4D-4F. The corresponding ¹H-NMR chemical shifts are listed in Table 9. The protons of the glycerol skeleton, —CH₂CH(O)CH₂— and —OCH₂CHCH₂O— are clearly present at δ 5.3-5.2 ppm and 4.4-4.1 ppm, respectively. Two kinds of double bonds were detected: (1) terminal double bond (n=0 in Scheme 3a), —CH═CH₂ and —CH═CH₂ present at δ 5.8 ppm and 5.0 to 4.9 ppm, respectively, and the internal double bond (n≠0 in Scheme 3a), —CH═CH— at δ 5.5 ppm to δ 5.3 ppm. The α-H to the ester group (—C(═O)CH₂—) was present at δ 2.33-2.28 ppm, α-H to —CH═CH— at δ 2.03-1.98 ppm, and —C(═O)CH₂CH₂— at δ 1.60 ppm. Two kind of —CH₃ were detected, one with n=2 (in Scheme 3a) at 1.0-0.9 ppm and another with n=8 at 0.9-0.8 ppm. It should be noticed that polyunsaturated fatty acids were not detected by NMR as the chemical shift at 2.6 to 2.8 ppm, the signature ¹H-NMR of the proton between two double bonds in a polyunsaturated fatty acid was not presented.

TABLE 9
¹H-NMR chemical shifts of SF-PMTAG and LF-PMTAG

| Proton | Chemical Shift (ppm) |
|---|---|
| —(CH₂)₇CH₃ | ~0.8-0.9 |
| —(CH₂)₂CH₃ | ~1.0 |
| —(CH₂)— | 1.4-1.2 |
| —CH₂CH₂COO— | ~1.6 |
| —CH₂CH═ | 2.1-1.9 |
| —CH₂COO— | 2.4-2.2 |
| —OCH₂CH(O)CH₂O— | 4.3-4.1 |
| —CH═CH₂ | 5.0-4.8 |
| —OCH₂CH(O)CH₂O— | 5.3-5.2 |
| —CH═CH— | 5.5-5.3 |
| —CH═CH₂ | ~5.8 |

Due to the very low content of free fatty acid in the MTAG material as indicated by the acid value (<1), the analysis was performed assuming that only TAG structures were present in the MTAG and in its fractions. The fatty acid profile of the MTAGs was calculated based on the relative area under the characteristic chemical shift peaks. The results are listed in Table 10.

The PMTAG fractions also contains saturated TAGs including PPP, PPM and PPS that exist in the starting natural oil. However, as indicated by ¹H-NMR, there are more internal double bond with oleyl structure and less saturated fatty acid chain in LF-PMTAG than in SF PMTAG (Table 10). Note that the amount of terminal double bonds and butyl terminal double bonds in LF(D1)-PMTAG and SF(D1)-PMTAG are similar. Also, as listed in Table 10, LF(S)-PMTAG contained significantly less saturated fatty acids than SF(S)-PMTAG, but more double bonds, including terminal, butyl end double bonds and oleyl end double bonds.

TABLE 10
Fatty acid profile of PMTAG, SF-PMTAG and LF-PMTAG
calculated based on the relative area under the characteristic
¹H-NMR chemical shift peaks

| | Fatty Acids with: | | | |
|---|---|---|---|---|
| | | | other non- | |
| | | | terminal | Saturated |
| | —CH═CH₂ | —CH═CHCH₂CH₃ | double bonds | fatty acid |
| PMTAG | 24.9 | 15.8 | 10.6-14.5 | 44.8-48.7 |

Solid Fractions

| | | | | |
|---|---|---|---|---|
| SF(D1)- | 20.7 | 13.2 | 13.5 | 53.2 |
| PMTAG | | | | |
| SF(D2)- | 15.5 | 13.5 | 18.1 | 52.9 |
| PMTAG | | | | |
| SF(S)- | 13.5 | 11.1 | 16.9 | 58.5 |
| PMTAG | | | | |

Liquid Fractions

| | | | | |
|---|---|---|---|---|
| LF(D1)- | 21.1 | 14.1 | 16.7 | 48.1 |
| PMTAG | | | | |
| LF(D2)- | 18.1 | 17.5 | 19.8 | 44.6 |
| PMTAG | | | | |
| LF(S)- | 18.1 | 15.9 | 20.3 | 45.7 |
| PMTAG | | | | |

### HPLC of PMTAG Fractions

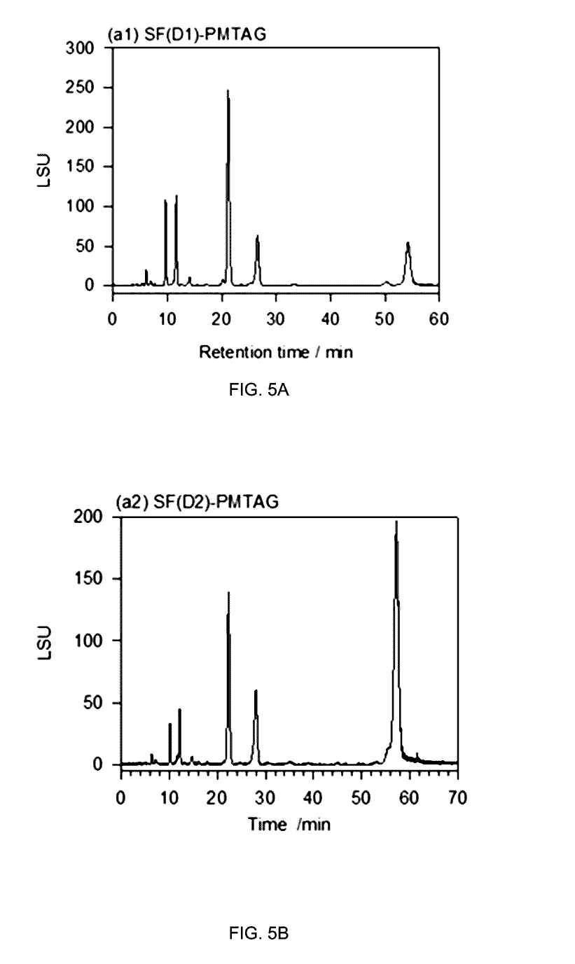

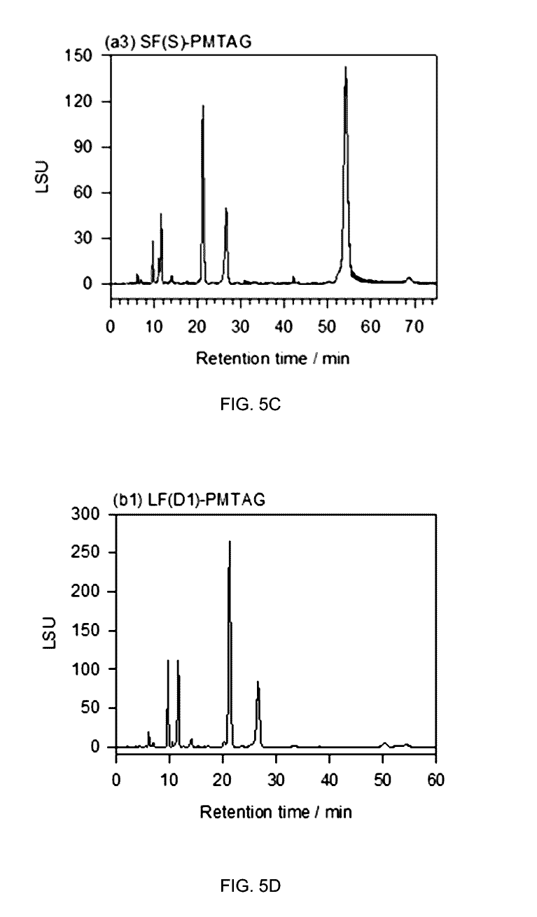

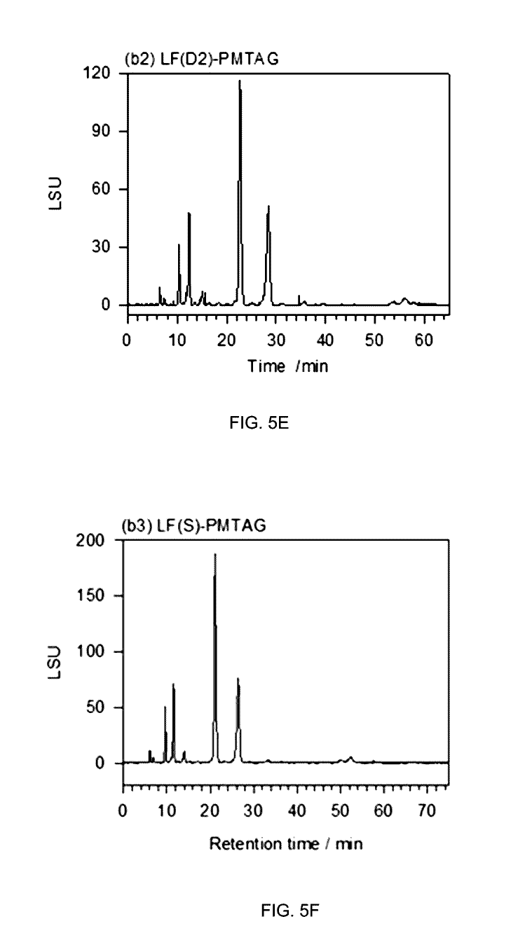

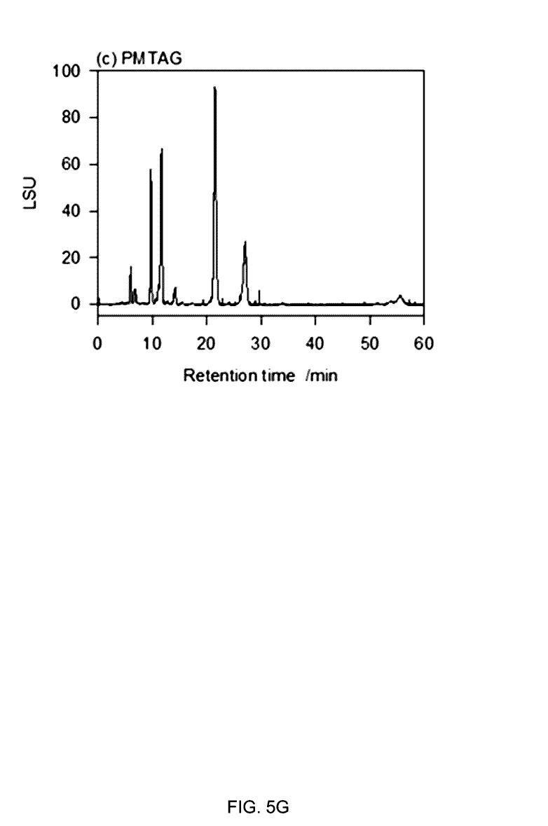

The HPLC curves of SF-PMTAG and LF-PMTAG are shown in FIGS. 5A-5F. The HPLC curve of PMTAG is presented in FIG. 5G for comparison purposes. As shown, an excellent separation was obtained. The analysis of the HPLC of the MTAG fractions was carried out with the help of standard curves of pure TAGs (DDD, DSS, DDS and PPP; D: 9-decenoic acid, S: Stearic acid, P: Palmitic acid) used as standards. The retention time of these standards were well matched with the related PMTAG fractions. The results of the analysis are reported in Table 11.

As listed in Table 11, the TAGs with shorter fatty acid chain, such as decenoic acid (C10) or lauroleic acid (C12), appeared at shorter retention times, those with longer fatty acid chain, such as palmitic acid (C16), stearic acid or oleic acid (C18), appeared at longer retention times. The HPLC results indicate that the types of TAGs present in PMTAG are also present in LF PMTAG and SF PMTAG but in different amounts. The main difference between SF-PMTAG and LF PMTAG is related to the TAGs eluting at ˜55 min, i.e., those with long chain fatty acids, including oleic, stearic and palmitic fatty acids. More TAGs eluted at ˜55 min, i.e., those with long chain fatty acids, including oleic, stearic and palmitic fatty acids, in SF-PMTAG than in LF-PMTAG. More TAGs with short fatty acids, such as decenoic acid (C10) or lauroleic acid (C12), were detected in LF-PMTAG than in SF-PMTAG.

TABLE 11
HPLC analysis data of PMTAG, SF(D1)-PMTAG and LF(D1)-
PMTAG. RT: Retention time (min). The content is based
on the relative area (Area %) under the HPLC peak.

| | SF(D1)-PMTAG | | SF(D2)-PMTAG | | SF(S)-PMTAG | | |
|---|---|---|---|---|---|---|---|
| | | Area | | Area | | Area | |
| Peak | RT | % | RT | % | RT | % | Structure |
| 1 | 5.4 | 0.15 | | | 6.1 | 0.51 | DDD |
| 2 | 6.0 | 1.59 | 6.3 | 0.57 | 6.9 | 0.28 | — |
| 3 | 6.9 | 0.61 | 7.2 | 0.35 | 9.67 | 2.33 | — |
| 4 | 9.7 | 8.42 | 10.2 | 2.02 | 10.6 | 0.09 | — |
| 5 | 10.4 | 0.19 | | | 11.6 | 6.44 | — |
| 6 | 11.6 | 11.86 | 12.2 | 4.42 | 14.0 | 0.91 | DDS |
| 7 | 14.1 | 1.47 | 14.8 | 0.81 | — | — | — |
| 8 | 17.1 | 0.36 | | | 17.6 | 0.13 | — |
| 9 | 20.1 | 1.28 | 21.3 | 0.27 | 20.2 | 0.15 | — |
| 10 | 21.2 | 39.02 | 22.3 | 18.47 | 21.2 | 19.38 | — |
| 11 | 25.2 | 0.56 | 28.0 | 11.63 | — | — | — |
| 12 | 26.5 | 14.09 | | | 26.5 | 11.74 | — |
| 13 | 33.4 | 0.44 | | | 32.9 | 0.26 | DSS |
| 14 | 50.3 | 1.35 | 57.2 | 61.45 | 50.2 | 0.30 | — |
| 15 | 54.4 | 18.60 | | | 54.2 | 56.17 | PPP |
| | | | | | 68.7 | 1.30 | |

| | PMTAG | | LF(D1)-PMTAG | | LF(D2)-PMTAG | | LF(S)-PMTAG | | |
|---|---|---|---|---|---|---|---|---|---|
| | | Area | | Area | | Area | | Area | |
| Peak | RT | % | RT | % | RT | % | RT | % | Structure |
| 1 | | | 5.4 | 0.15 | | | | | DDD |
| 2 | 6.0 | 3.18 | 6.1 | 1.82 | 6.5 | 1.4 | 6.1 | 1.21 | — |
| 3 | 6.9 | 1.72 | 6.9 | 0.72 | 7.3 | 0.83 | 6.8 | 0.70 | — |
| 4 | 9.7 | 11.73 | 9.7 | 9.72 | 10.4 | 5.82 | 9.6 | 5.69 | — |
| 5 | 10.6 | 0.32 | 10.5 | 0.23 | | | 10.5 | 0.26 | — |
| 6 | 11.1 | 1.93 | 11.6 | 12.81 | 12.4 | 12.67 | 11.5 | 11.20 | DDS |
| 7 | 11.7 | 17.75 | 14.0 | 1.73 | 13.5 | 0.26 | 14.0 | 2.13 | — |
| 8 | 12.9 | 0.48 | 17.1 | 0.28 | 15.1 | 2.23 | 15.2 | 0.38 | — |
| 9 | 14.3 | 2.82 | 20.3 | 1.22 | 21.7 | 0.63 | 17.0 | 0.49 | — |
| 10 | 15.5 | 0.28 | 21.2 | 46.64 | 22.7 | 42.43 | 20.2 | 0.56 | — |
| 11 | 17.4 | 0.59 | 25.2 | 0.64 | 28.4 | 28.53 | 21.1 | 44.68 | — |
| 12 | 20.7 | 0.31 | 26.5 | 20.63 | | | 23.3 | 0.44 | — |
| 13 | 21.5 | 37.60 | 33.4 | 0.36 | 53.9 | 1.25 | 26.4 | 26.40 | DSS |
| 14 | 27.0 | 16.23 | 50.3 | 1.94 | 56.0 | 3.90 | 33.2 | 1.06 | — |
| 15 | 55.5 | 4.98 | 54.4 | 1.74 | | | 50.0 | 1.46 | PPP |
| 16 | | | | | | | 53.8 | 3.35 | |

### Physical Properties of PMTAG Fractions

### Thermal Degradation of PMTAG Fractions

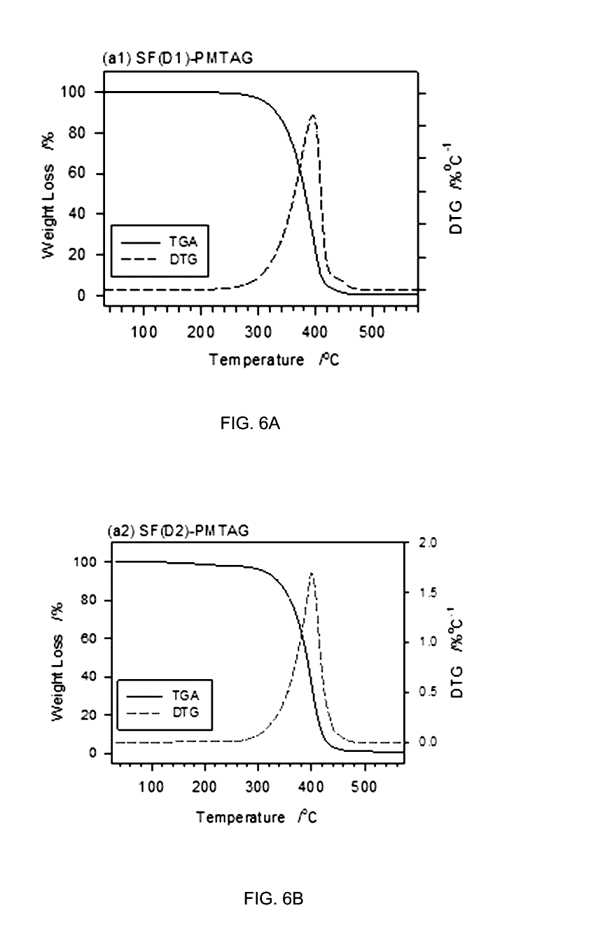

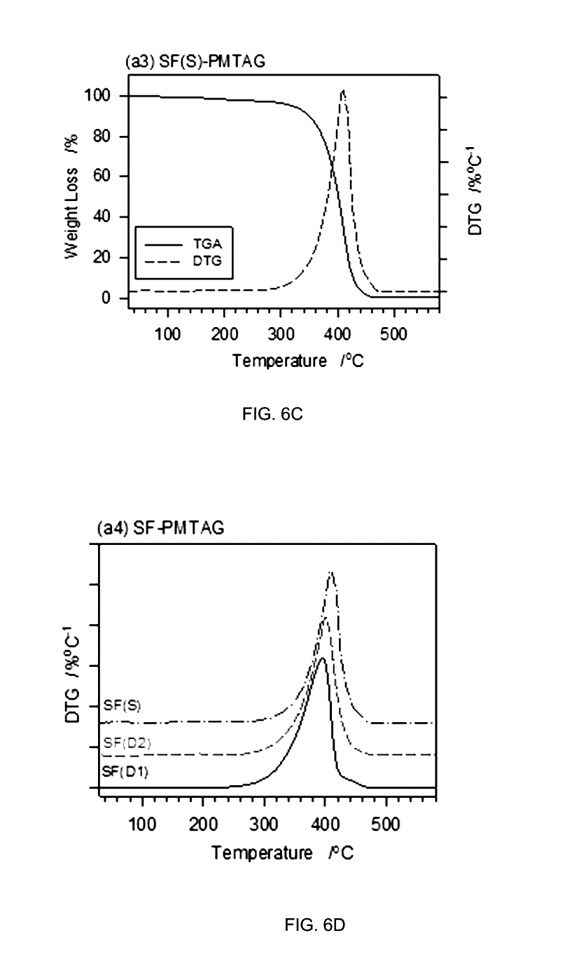

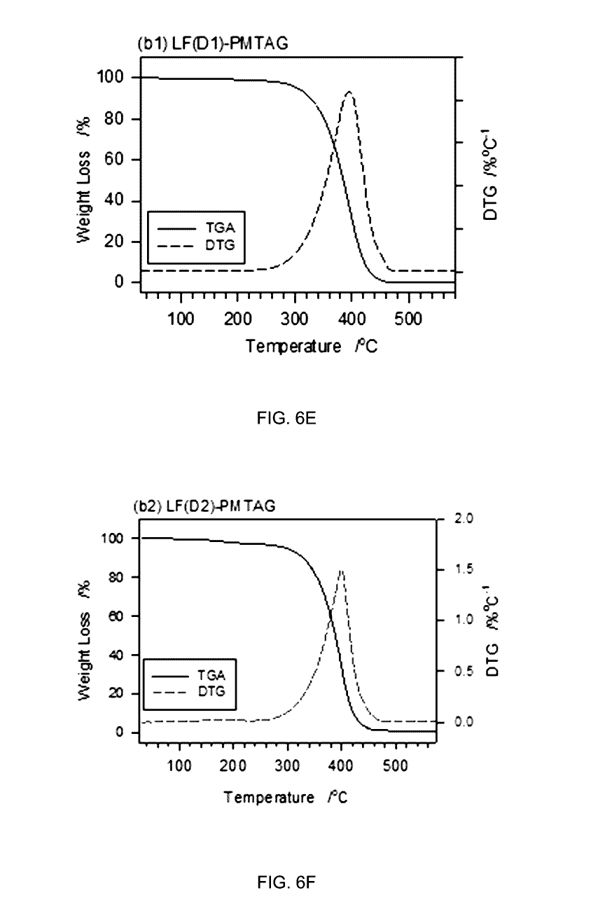

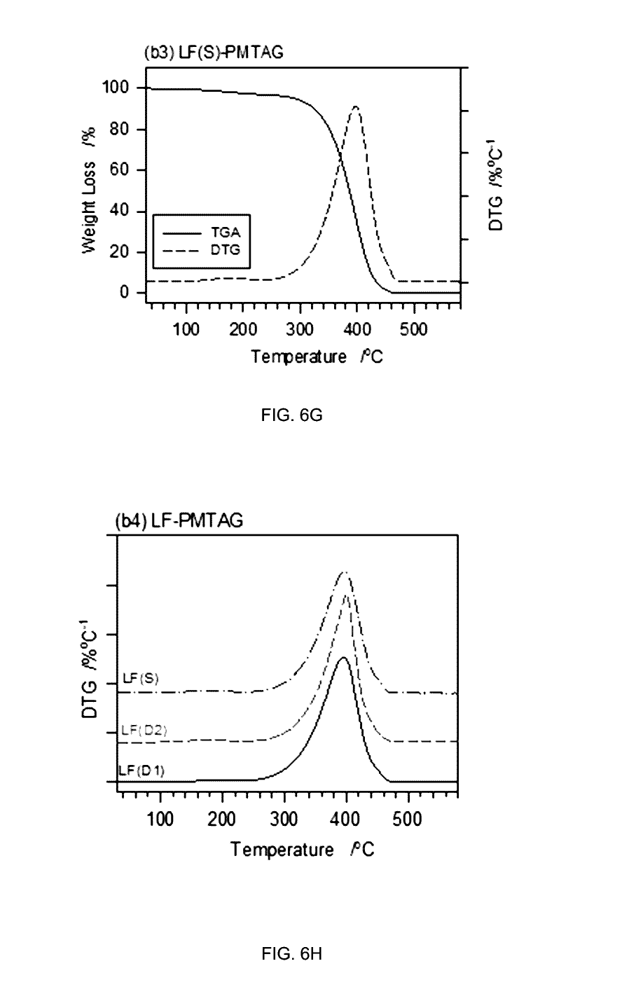

The TGA and DTG profiles of SF-PMTAG and LF-PMTAG are shown in FIGS. 6A-6H. The corresponding data (onset of degradation of PMTAG fractions as measured by the temperature at 1, 5 and 10% decomposition and DTG peak temperatures) are listed in Table 12.

TGA and DTG reveal one main decomposition mechanism for the PMTAG fractions, associated with the breakage of the ester bonds. The onset of thermal degradation of the solid fraction as determined at 5% weight loss and extrapolated decomposition onset temperature are higher than those of the liquid fraction and the PMTAG itself (see Table 12), probably due to differences in evaporation. Although the solid and liquid fractions of the MTAG presented different decomposition rates at the DTG peak (D1: 1.60 and 1.26%/° C., respectively; D2: 1.70 and 1.50%/° C., respectively; S: 1.87 and 1.23%/° C., respectively), the DTG peaks (both at 400 C) and offset temperatures at ˜422° C., indicate a relatively similar thermal stability. Note that the thermal stability of the MTAG fractions is relatively higher than common commercial vegetable oils, such as olive, canola, sunflower and soybean oils, for which first DTG peaks show at temperature as low as 325° C.

As can be seen from the TGA and DTG curves, the decompositions of SF(S)-PMTAG and LF(S)-PMTAG have extrapolated onset temperatures of 376 and 346° C., respectively, and end at 467 and 470° C., respectively. Furthermore, at the DTG peak, the liquid and solid fraction of the MTAG lost nearly 63 wt % with rates of degradation of 1.23 and 1.87%°/C., respectively.

TABLE 12
Temperature of degradation at 1, 5 and 10% weight loss (T_{1%}^{d}, T_{5%}^{d},
T_{10%}^{d}, respectively), DTG peak temperatures (T_{D1-2}) and weight loss at T_{D1-2}
of PMTAG and PMTAG fractions obtained by dry crystallization (rates method
(D1) and quiescent method (D2)) and solvent aided crystallization method (S)

| | Temperature (° C.) | | | | | | | Weight Loss (%) at | |
|---|---|---|---|---|---|---|---|---|---|
| Material | T_{1%}^{d}, | T_{5%}^{d} | T_{10%}^{d} | T_{D1} | T_{D2} | Tₒₙ | T_{off} | T_{D1} | T_{D2} |
| PMTAG | 260 | 309 | 330 | | 399 | | | | 62 |
| SF(D1)- | 259 | 311 | 330 | 182 | 395 | 327 | 415 | 0.2 | 70 |
| PMTAG | | | | | | | | | |
| SF(D2)- | 183 | 312 | 337 | 192 | 400 | 337 | 423 | 1 | 64 |
| PMTAG | | | | | | | | | |
| SF(S)- | 141 | 319 | 349 | 196 | 409 | | | 2 | 63 |
| PMTAG | | | | | | | | | |
| LF(D1)- | 207 | 305 | 328 | 189 | 395 | 326 | 431 | 0.8 | 63 |
| PMTAG | | | | | | | | | |
| LF(D2)- | 163 | 299 | 329 | 178 | 400 | 343 | 421 | 2 | 66 |
| PMTAG | | | | | | | | | |
| LF(S)- | 137 | 291 | 324 | 178 | 398 | | | 2 | 63 |
| PMTAG | | | | | | | | | |

### Crystallization and Melting Behavior of PMTAG Fractions

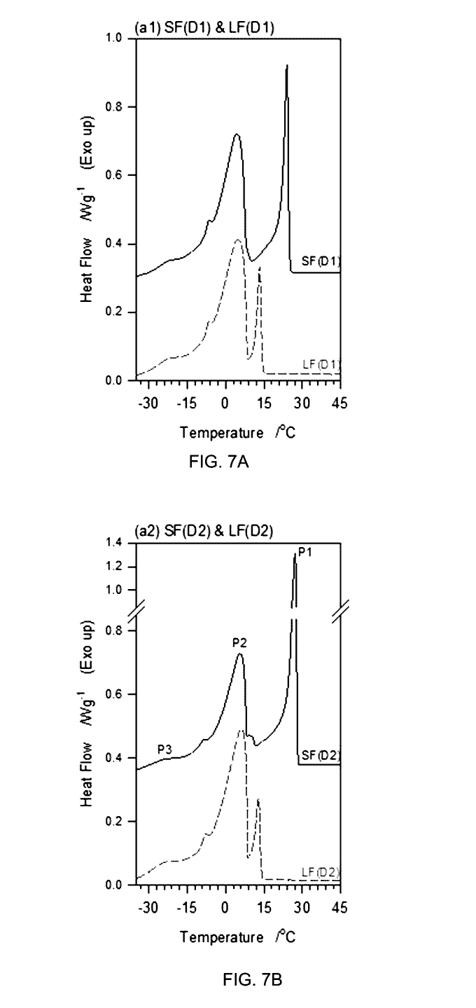

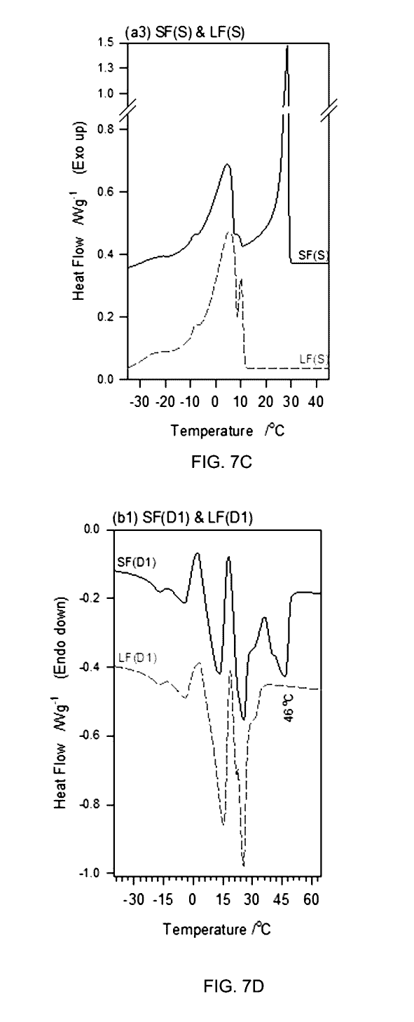

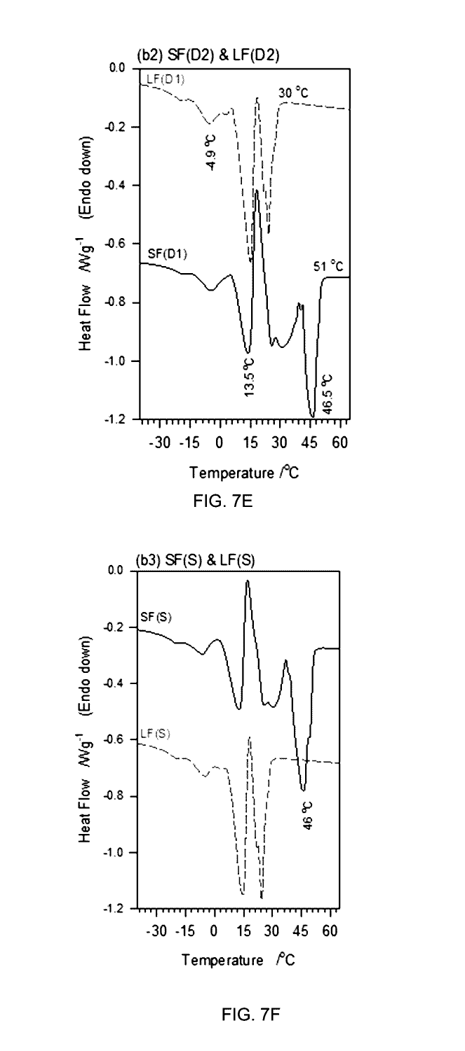

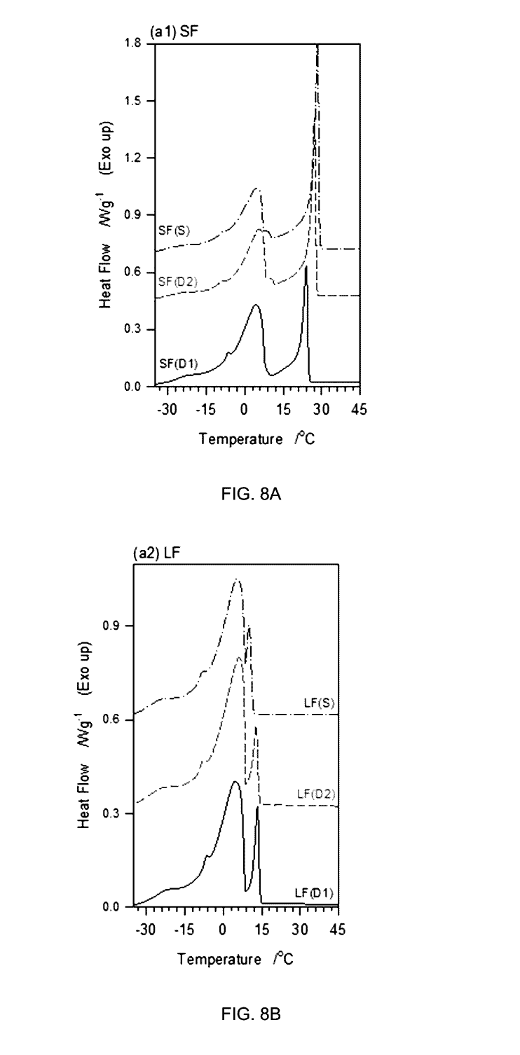

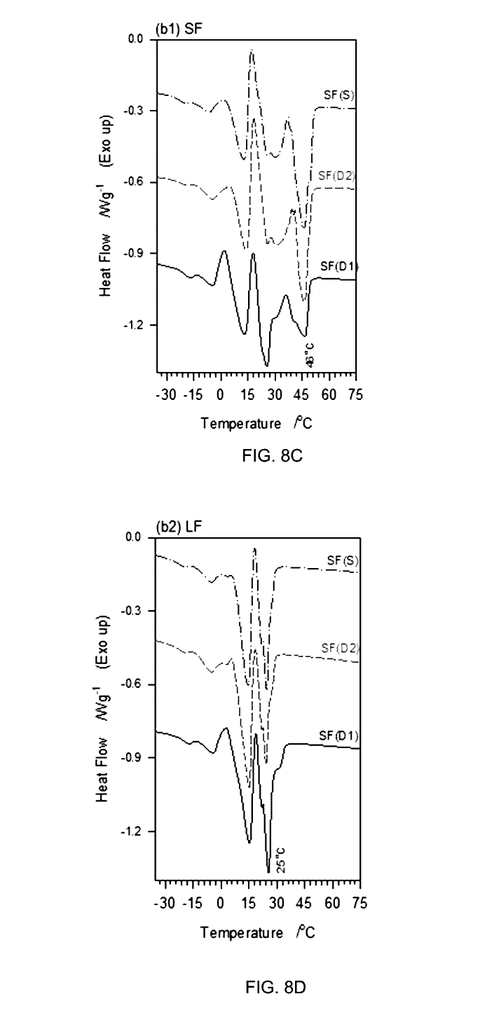

The DSC thermograms of the PMTAG liquid and solid fractions obtained on cooling and subsequent heating (both at 5° C./min) are presented in FIGS. 7A-7F, respectively. The corresponding thermodynamic data are listed in Table 13.

Both the solid and liquid fractions of PMTAG presented three exotherms (FIGS. 7A-7C) which were presented by the PMTAG, indicating that both have stearin and olein components. Note however, that the portions of the MTAG are not exactly the same as those of the native palm oil and are named in this manner for convenience. In fact the PMTAG fractions contain trans- and cis-, as well as short and long chains that have more complex crystallization behavior than the starting palm oil material.

At least five endotherms and one or two resolved exotherms were observed in their DSC heating thermograms indicating that both the solid and liquid fractions are polymorphic. However, the cooling thermograms of the liquid fractions presented a shift to sub ambient temperature of their leading exotherm, and their heating thermograms were missing the highest melting peak at 46-47° C. (in FIGS. 7D-7F). This indicates that the stearin portion of the liquid fraction was depleted from the most high crystallizing components of PMTAG.

Note that the onset of crystallization of LF(S)-PMTAG shifted the most (˜11° C. compared to ˜14° C. for LF(D1)-PMTAG and ˜18° C. for LF(D2)-PMTAG, Table 13) and its heating thermogram did not show two of the highest melting peaks that were present in the heating thermogram of PMTAG (peaks at 30 and 46° C. in FIGS. 7D-7F). Furthermore, the enthalpy of crystallization of the stearin components in the liquid fractions obtained with method D1 and D2 is ˜⅓ of that of the solid fraction counterparts, and the enthalpy of the stearin part in LF(S)-PMTAG is approximately a tenth of that of SF(S)-PMTAG. Also, the enthalpy of melting as determined from the endotherms of the solid fraction, was much higher than that of the liquid fraction (152.7 vs 80.5 J/g in D1; 140 vs 103.4 J/g in D2, and 110.2 vs 78.8 J/g in S), reflecting the imbalance in composition between the two fractions.

TABLE 13
Thermal data of SF- and LF-PMTAG. Tₒₙ, T_{off}, T₁₋₃: onset, offset
and peak temperatures (° C.), ΔH_{S}, ΔH_{O}, and ΔH (J/g):
Enthalpy of the stearin and olein portions, and total enthalpy, respectively.
Cooling cycle (5° C./min)

| | T (° C.) | | Exotherms | | | Enthalpy (J/g) | | |
|---|---|---|---|---|---|---|---|---|
| | Tₒₙ | T_{off} | T₁ | T₂ | T₃ | ΔH_{S} | ΔH_{O} | ΔH |
| SF(D1)- PMTAG | 24.88 | −31.54 | 23.97 | 4.21 | −21.31 | 26 | 64 | 90 |
| SF(D2)- PMTAG | 27.98 | −37.81 | 27.12 | 5.51 | −22.86 | 39 | 51 | 90 |
| SF(S)- PMTAG | 29.09 | −36.24 | 28.39 | 4.47 | −21.66 | 45.3 | 48.0 | 93.3 |
| LF(D1)-PMTAG | 14.31 | −31.46 | 13.31 | 4.88 | −21.65 | 9 | 61 | 70 |
| LF(D2)-PMTAG | 13.85 | −36.46 | 12.83 | 6.37 | −23.79 | 9 | 71 | 80 |
| LF(S)-PMTAG | 11.45 | −37.19 | 10.03 | 5.50 | −23.36 | 7.2 | 69.2 | 76.4 |

Heating cycle (5° C./min)

| | T (° C.) | | Endotherms | | | | | Exotherms | |
|---|---|---|---|---|---|---|---|---|---|
| | Tₒₙ | T_{off} | T₁ | T₂ | T₃ | T₄ | T₅ | T_{R1} | T_{R2} |
| SF(D1)-PMTAG | −25.33 | 49.31 | 46.55 | 25.65 | 13.31 | −4.60 | −17.24 | 18.00 | 2.29 |
| ΔH (J/g) | — | — | 28.4 | 92.7 | 20.7 | 10.9 | | 4.9 | 2.5 |
| SF(D2)-PMTAG | −26.68 | 50.44 | 46.53 | 31.46 | 13.52 | −4.85 | −18.38 | 18.35 | — |
| ΔH (J/g) | — | — | 36 | 39 | 18.6 | 9.8 | | 4.9 | 12.4 |
| SF(S) -PMTAG | −28.23 | 51.29 | 46.34 | 30.25 & | 12.77 | −6.61 | −20.32 | 1.49 | 17.08 |
| | | | | 25.65 | | | | | |
| ΔH (J/g) | | | 52.2 | 29.4 | 18.9 | 9.7 | | 0.2 | 9.6 |
| LF(D1) -PMTAG | −25.62 | 34.53 | 31.56 | 25.56 | 15.38 | −4.36 | −17.25 | 18.68 | 3.06 |
| ΔH (J/g) | | | shoulder | 37.9 | 33.6 | 9.0 | | 0.4 | 2.4 |
| LF(D2) -PMTAG | −31.26 | 28.95 | 27.77 | 25.56 | 15.12 | −4.90 | −19.56 | 18.35 | — |
| ΔH (J/g) | — | — | shoulder | 70 | 42 | 28 | | 0.2 | — |
| LF(S) -PMTAG | −28.17 | 29.14 | 27.49 | 24.50 | 14.79 | −5.18 | −19.47 | — | 18.04 |

| | | | | | | | | |
|---|---|---|---|---|---|---|---|---|
| ΔH (J/g) | — | — | shoulder | 30.5 | 34.7 | 13.6 | — | 1.2 |

### Solid Fat Content of PMTAG Fractions

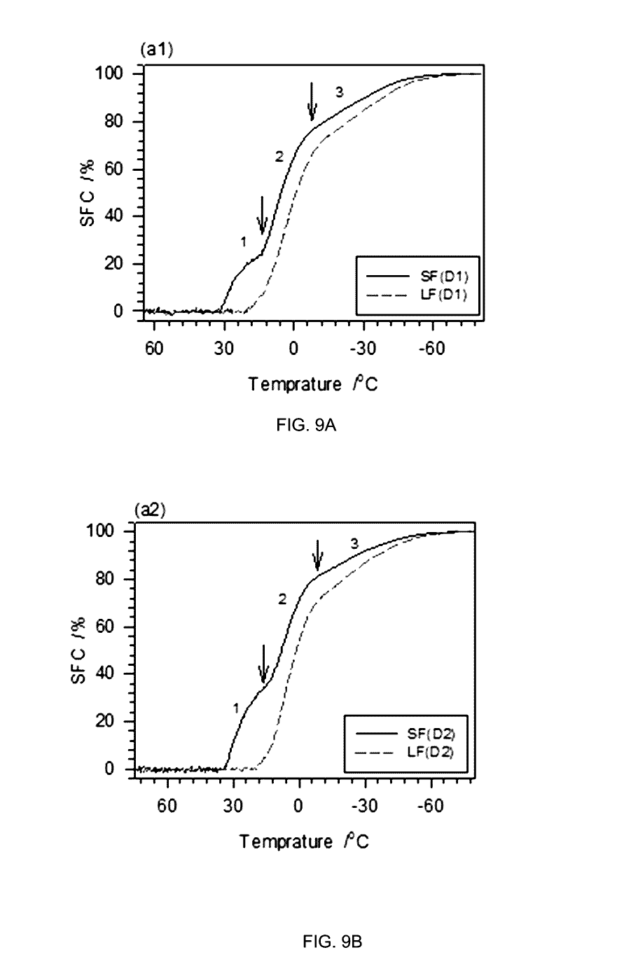

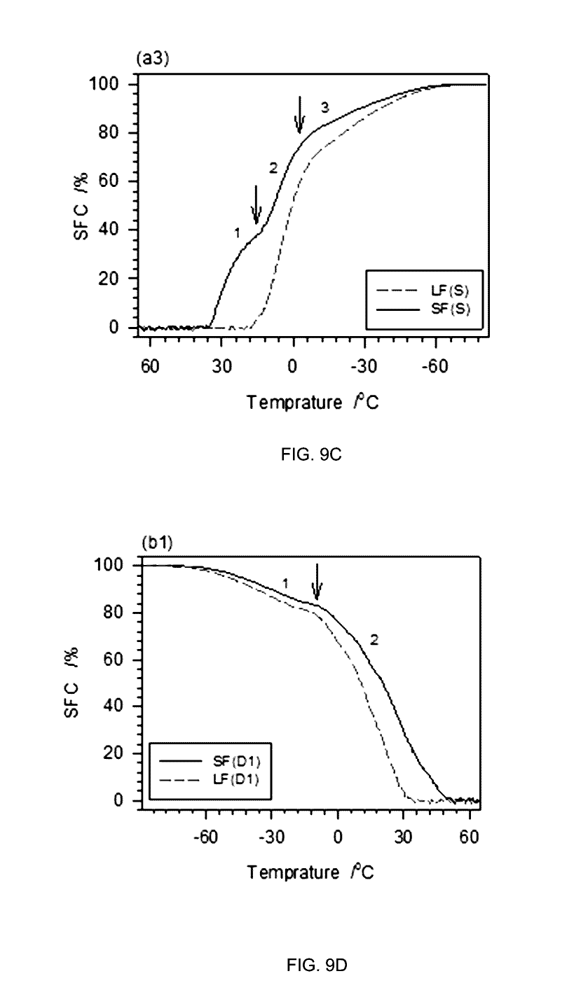

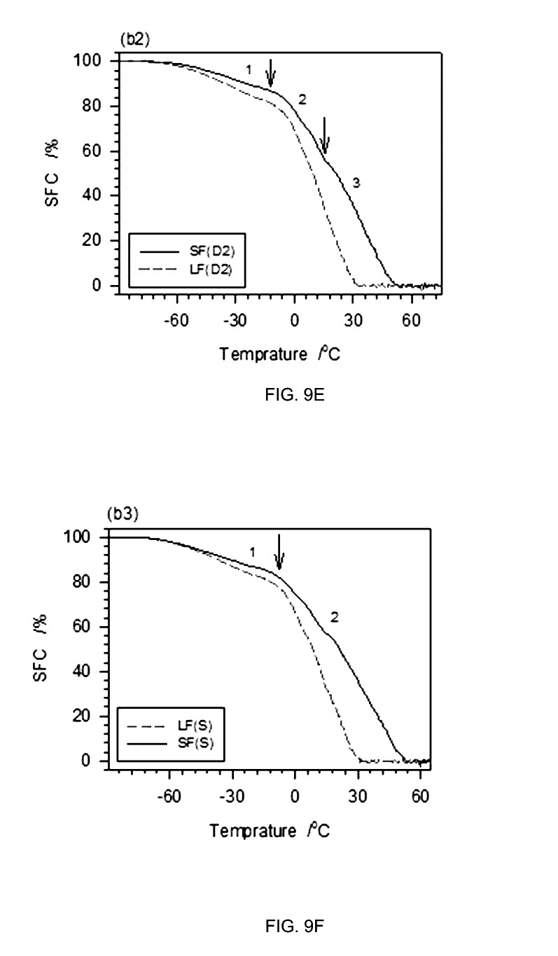

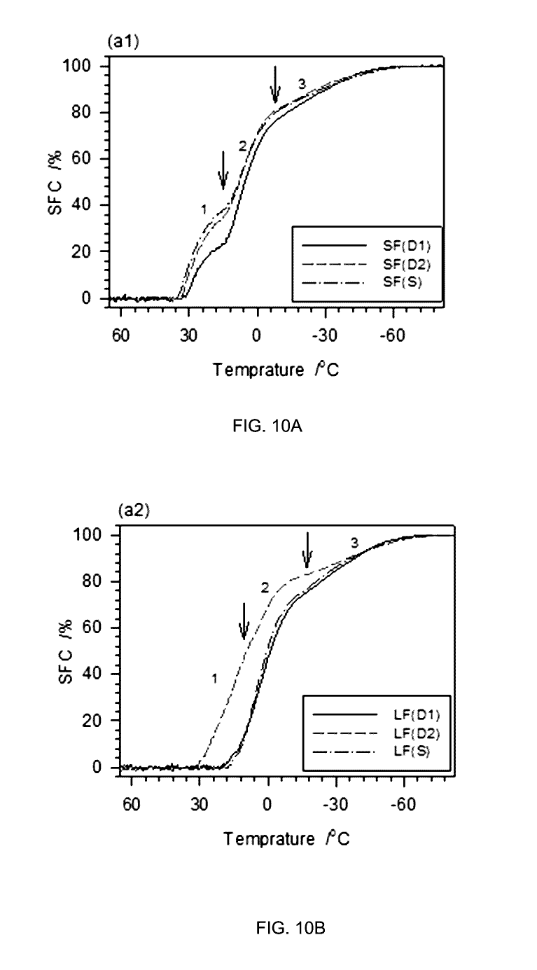

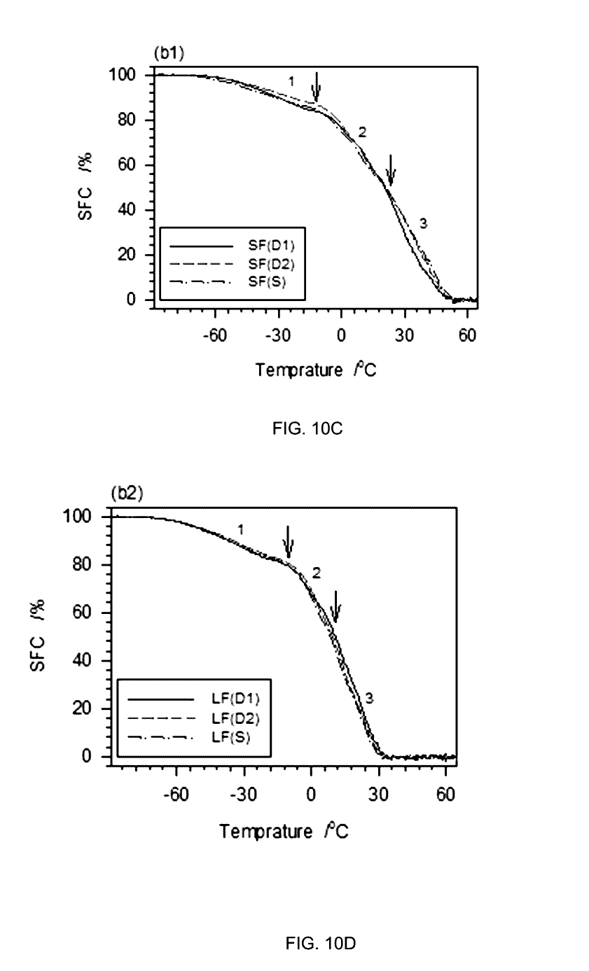

Solid Fat Content (SFC) versus temperature curves of PMTAG fractions obtained during cooling (5° C./min) and heating (5° C./min) are shown in FIGS. 9A-9F, respectively. The extrapolated induction and offset temperatures as determined by SFC are listed in Table 14. As can be seen in FIGS. 9A-9C, the SFC cooling curves of both solid and liquid fractions presented three segments indicative of a three-step solidification process. In each fraction, the first segment (segment 1 in FIGS. 9A-9C) is associated with the solidification of the stearin portion and the two others (segments 2 and 3 in FIGS. 9A-9C) to the olein portion. Noticeably, as indicated by its much more considerable first SFC segment, SF-PMTAG has a larger PMTAG stearin portion than LF-PMTAG. Note that the SFC heating curves of both solid and liquid fractions presented only two identifiable segments (segments 1 and 2 in FIGS. 9D-9F) associated with the melting of two different portions in each fraction.

Also, SF(D1)-PMTAG presented induction and melting temperatures (31.5 and 49.7° C., respectively) higher than LF(D1)-MTAG (19.4 and 31.6° C., respectively) similar to what was observed in the DSC. SF(D2)-PMTAG presented an SFC induction temperature (34.3° C.) higher than LF(D2)-MTAG (19.2° C.) similar to what was observed in the DSC. SF(S)-PMTAG presented induction and melting temperatures (34.8 and 51.2° C., respectively) higher than LF(S)-MTAG (17.1 and 29.8° C., respectively) similar to what was observed in the DSC.

TABLE 14
Extrapolated induction and offset temperatures (T_{ind},
Tₛ, respectively) of SF- and LF-PMTAG as determined by SFC

| Temperature (° C.) | |
|---|---|
| Cooling | Heating |

| | T_{ind} | Tₛ | T_{ind} | Tₛ |
|---|---|---|---|---|
| SF(D1)-PMTAG | 31.5 | −49.2 | −56.6 | 49.7 |
| SF(D2)-PMTAG | 34.3 | −63.2 | −60.1 | 51.5 |
| SF(S)-PMTAG | 34.8 | −50.6 | −65.8 | 51.2 |
| LF(D1)-PMTAG | 19.4 | −53.6 | −61.2 | 31.6 |
| LF(D2)-PMTAG | 19.2 | −63.1 | −62.2 | 30.9 |
| LF(S)-PMTAG | 17.1 | −56.4 | −62.1 | 29.8 |

### Flow Behavior and Viscosity of PMTAG

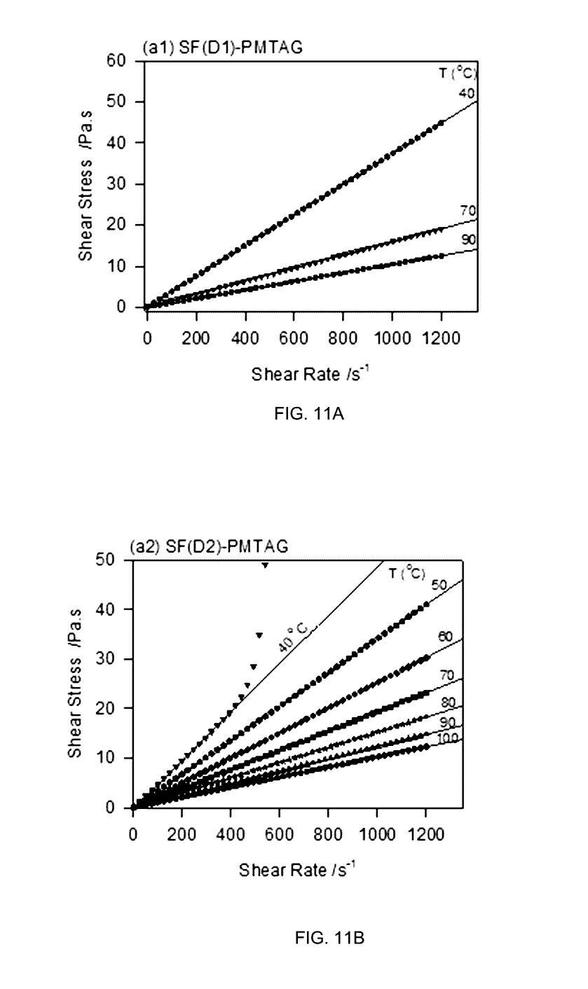

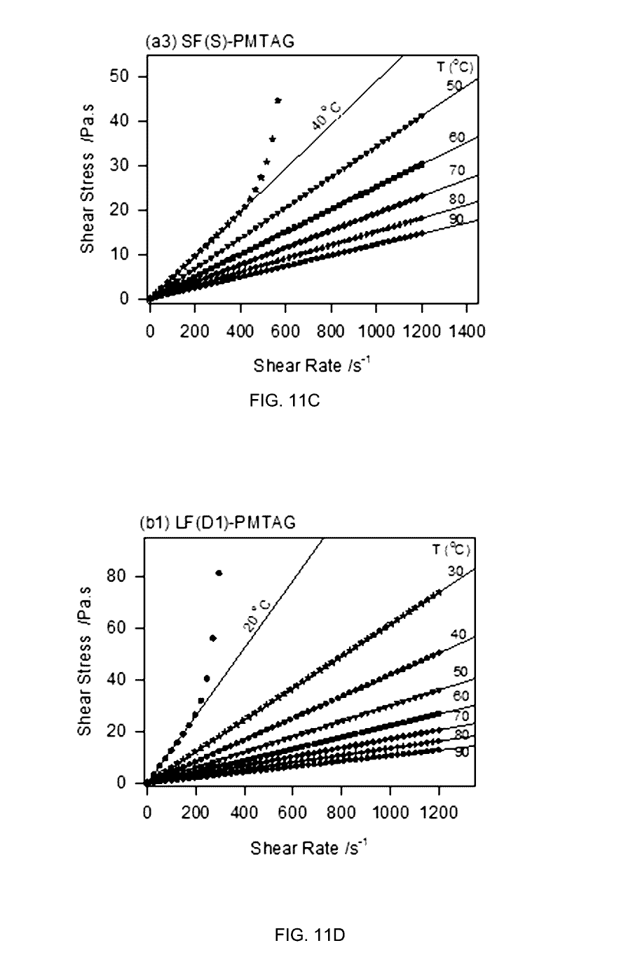

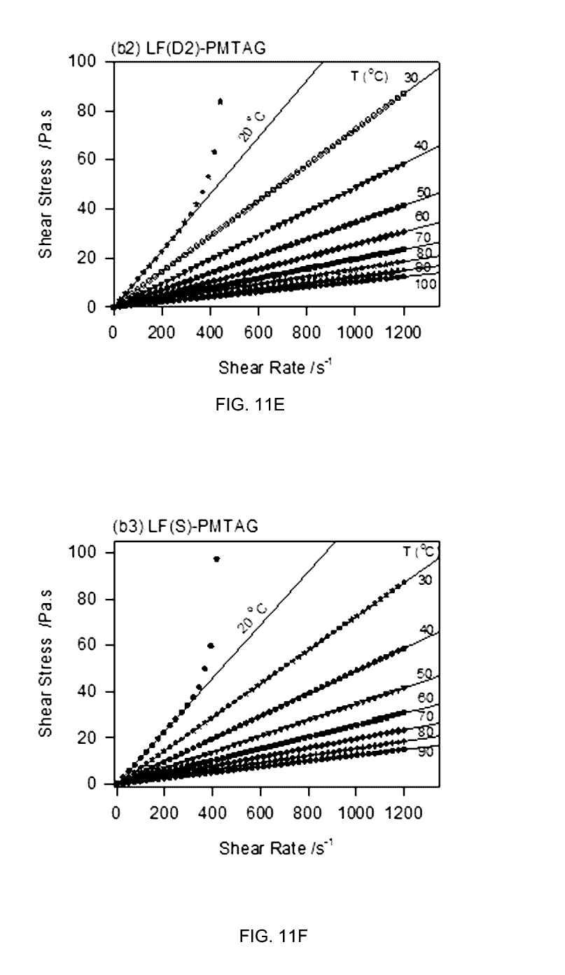

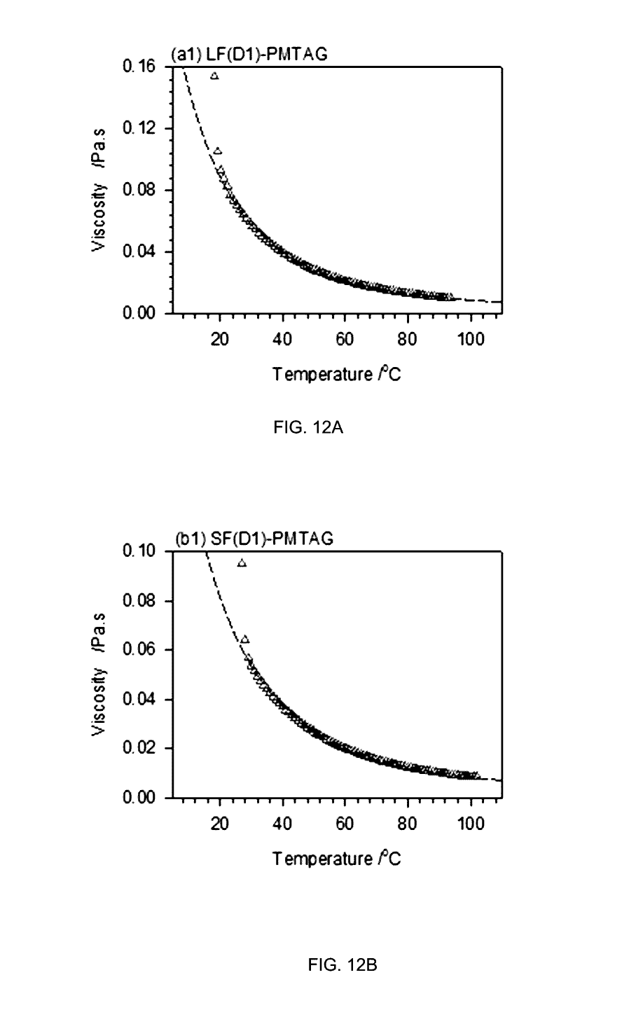

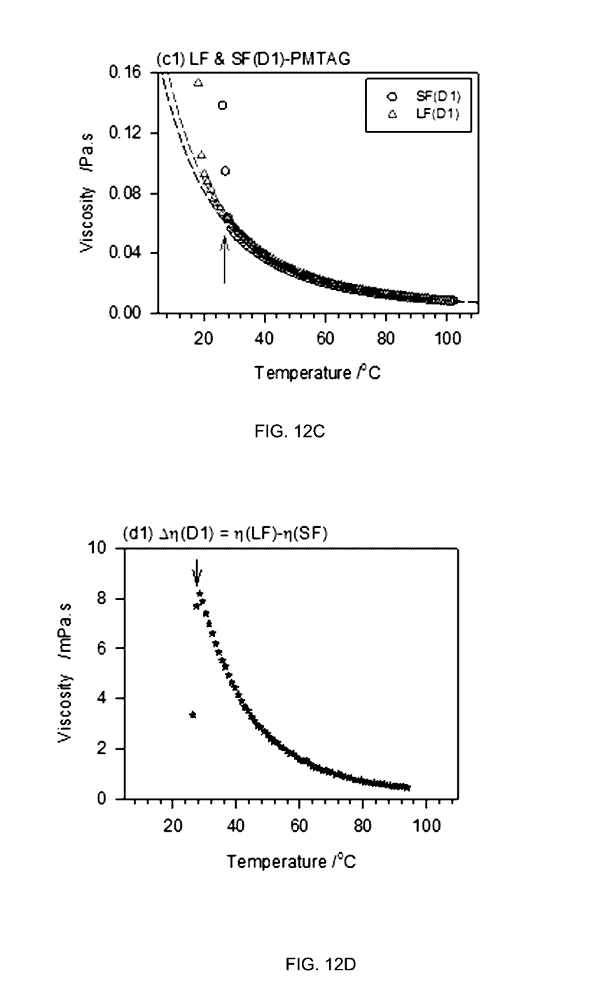

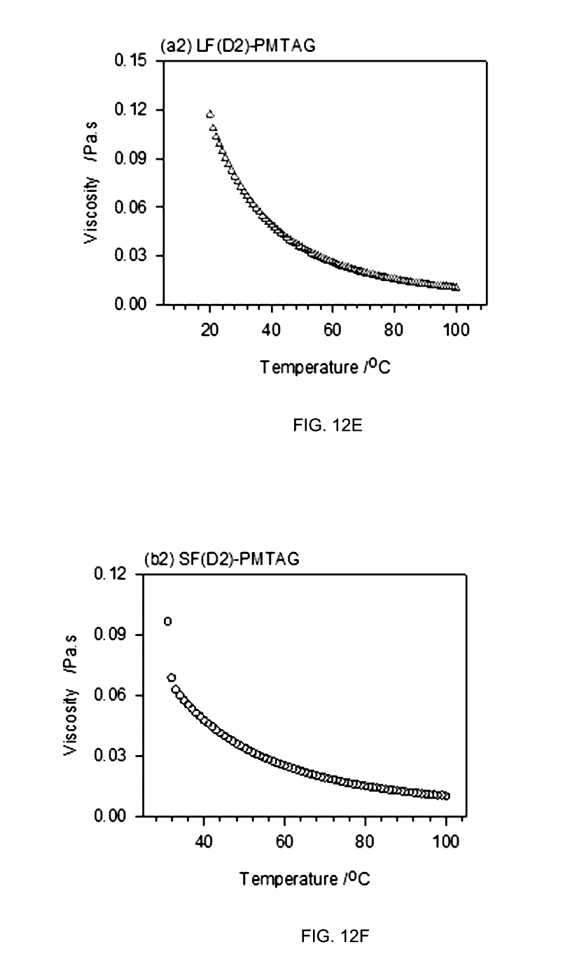

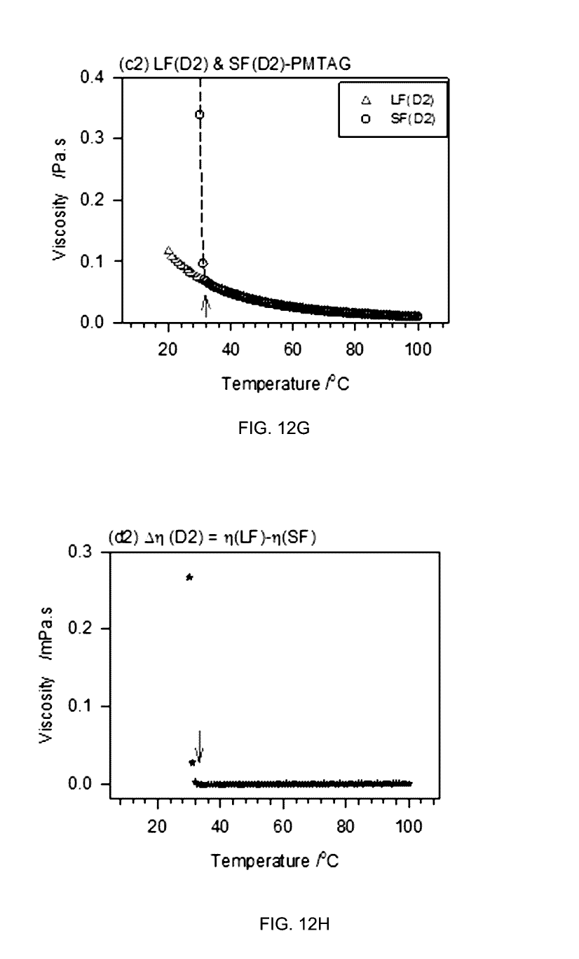

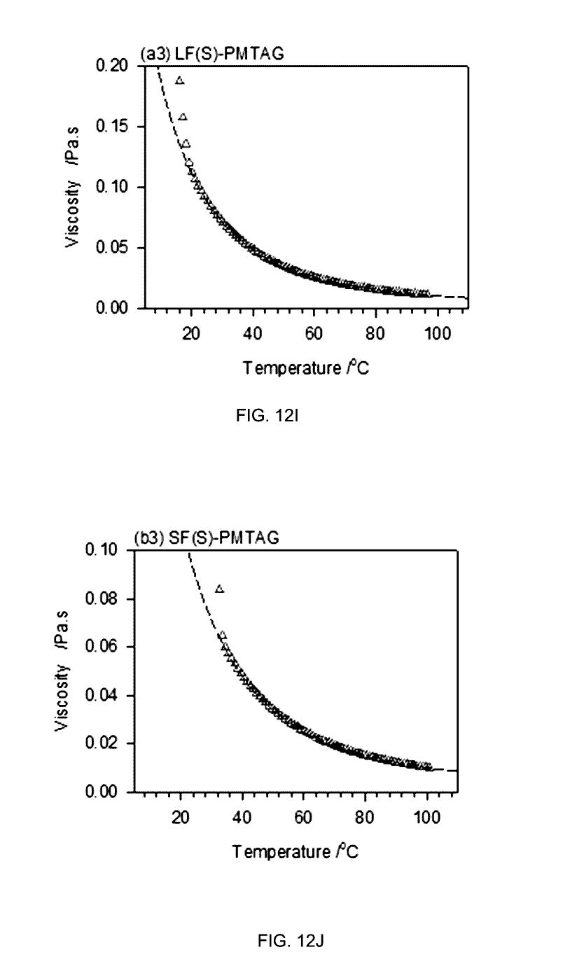

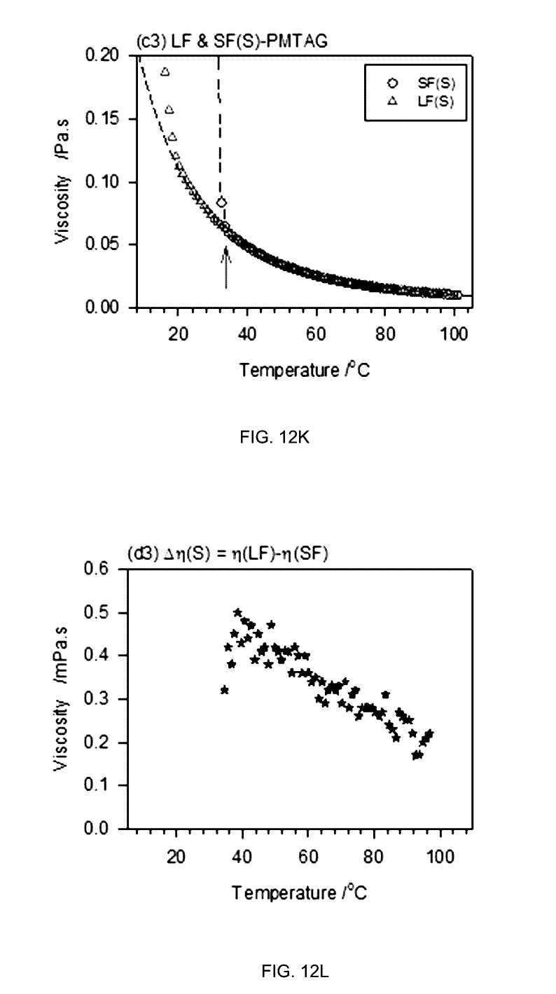

Selected shear rate—shear stress curves of the solid and liquid fractions of palm oil MTAG are displayed in FIGS. 11A-11F. Fits to the Herschel-Bulkley (eq. 1) model are included in FIGS. 11A-11F. FIGS. 12A, 12B, 12E, 12F, 12I, and 12J show their viscosity versus temperature curves obtained during cooling. Viscosity versus temperature curves of the solid and liquid fractions of palm oil MTAG are compared in FIGS. 12C, 12G, and 12K, and their difference

(Δη) is shown in FIGS. 12D, 12H, and 12L.

As can be seen in FIGS. 11A-11F, for the whole range of shear rates used, LF-PMTAG and SF-PMTAG presented a Newtonian behavior at temperatures above 20 and 40° C., respectively. The application of the Herschel-Bulkley equation (Eq. 1) to share rate—shear stress data in the Newtonian region at temperatures above the crystallization temperature generated power index values (1) all approximately equal to unity and no yield stress (Straight Lines in FIGS. 11A-11F, R²>0.99999).

The viscosity versus temperature of both fractions of PMTAG (FIGS. 12A, 12B, 12E, 12F, 12I, and 12J) presented the typical exponential behavior of liquid hydrocarbons. Note that the viscosity of the solid fraction of PMTAG was higher than that of the liquid fraction for temperatures higher than the crystallization temperature of the solid fraction only (Ton of SF(D1)-PMTAG ˜25° C., SF(S)-PMTAG ˜30° C.). For temperatures lower than Ton, the viscosity difference decreased exponentially from 8.2 mPa·s to 0.5 mPa·s for the (D1) fractions (FIG. 12D), whereas, the viscosity of the liquid and solid fractions (D2) and (S) differed by less than 0.5 mPa·s (FIGS. 12H and 12L).

### Comparison of Viscosity of Dry and Solvent Fractions

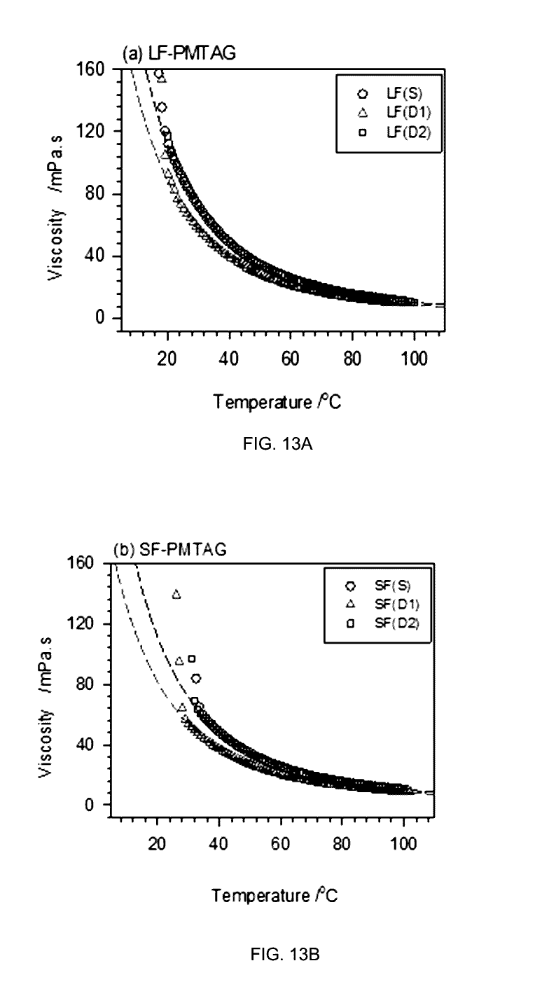

Viscosity versus temperature graphs of LF(S)-PMTAG, LF(D1)-PMTAG and LF(D2)-PMTAG are shown in FIG. 13. As can be seen in FIGS. 13A and 13B, both solid and liquid fractions of PMTAG obtained by solvent fractionation (S) displayed similar viscosities to their dry crystallization quiescent method (D2) counterparts, and higher than their dry crystallization rates method (D1) at all measurement temperatures. The difference which is as high as ˜20 mPa·s at Ton (24° C. for LF, and 34° C. for SF) decreased exponentially with increasing temperature to reach 8-10 mPa·s at 45° C. and 1.5 mPa·s at 100° C. (FIGS. 13C and 13D).

Polyols from Fractions of PMTAG
Note: A description of the PMTAG polyol synthesis with and without solvent is provided. Polyols from the fraction obtained with methods D1 and S were synthesized with the method using solvent, and polyols from the fractions from D2 were synthesized with the method without solvent.
Synthesis of Polyols from PMTAG Fractions

The synthesis of the Polyols from the liquid and solid fractions of MTAG of Palm Oil (LF-PMTAG Polyol and SF-PMTAG Polyol) involves epoxidation and subsequent hydroxylation of the liquid and solid fractions of MTAG of a natural oil. Any peroxyacid may be used in the epoxidation reaction, and this reaction will convert a portion of or all of the double bonds present in the MTAG to epoxide groups. Peroxyacids (peracids) are acyl hydroperoxides and are most commonly produced by the acid-catalyzed esterification of hydrogen peroxide. Any suitable peroxyacid may be used in the epoxidation reaction. Examples of hydroperoxides that may be used include, but are not limited to, hydrogen peroxide, tert-butylhydroperoxide, triphenylsilylhydroperoxide, cumylhydroperoxide, trifluoroperoxyacetic acid, benzyloxyperoxyformic acid, 3,5-dinitroperoxybenzoic acid, m-chloroperoxybenzoic acid and preferably, hydrogen peroxide. The peroxyacids may be formed in-situ by reacting a hydroperoxide with the corresponding acid, such as formic or acetic acid. Other organic peracids may also be used, such as benzoyl peroxide, and potassium persulfate. The epoxidation reaction can be carried out with or without solvent. Commonly used solvents in the epoxidation of the present invention may be chosen from the group including but not limited to aliphatic hydrocarbons (e.g., hexane and cyclohexane), organic esters (i.e. ethyl acetate), aromatic hydrocarbons (e.g., benzene and toluene), ethers (e.g., dioxane, tetrahydrofuran, ethyl ether, tert-butyl methyl ether) and halogenated hydrocarbons (e.g., dicholoromethane and chloroform).

Subsequent to the epoxidation reaction, the reaction product may be neutralized. A neutralizing agent may be added to neutralize any remaining acidic components in the reaction product. Suitable neutralizing agents include weak bases, metal bicarbonates, or ion-exchange resins. Non-limiting examples of neutralizing agents that may be used include ammonia, calcium carbonate, sodium bicarbonate, magnesium carbonate, amines, and resin, as well as aqueous solutions of neutralizing agents. Subsequent to the neutralization, commonly used drying agents may be utilized. Such drying agents include inorganic salts (e.g. calcium chloride, calcium sulfate, magnesium sulfate, sodium sulfate, and potassium carbonate).

After the preparation of the epoxidized MTAG, the next step is to ring-open at least a portion of the epoxide groups via a hydroxylation step. In the present work, all the epoxide groups were opened. The hydroxylation step consists of reacting the oxirane ring of the epoxide in an aqueous or organic solvent in the presence of an acid catalyst in order to hydrolyze the oxirane ring to a dihydroxy intermediate. In some aspects, the solvent may be water, aliphatic hydrocarbons (e.g., hexane and cyclohexane), organic esters (i.e. ethyl acetate), aromatic hydrocarbons (e.g., benzene and toluene), ethers (e.g., dioxane, tetrahydrofuran, ethyl ether, tert-butyl methyl ether) and halogenated hydrocarbons (e.g., dicholoromethane and chloroform), preferably water and/or tetrahydrofuran. The acid catalyst may be an acid such as sulfuric, pyrosulfuric, perchloric, nitric, halosulfonic acids such as fluorosulfonic, chlorosulfonic or trifluoromethane sulfonic, methane sulfonic acid, ethane sulfonic acid, ethane disulfonic acid, benzene sulfonic acid, or the benzene disulfonic, toluene sulfonic, naphthalene sulfonic or naphthalene disulfonic acids, and preferably perchloric acid. As needed, subsequent washing steps may be utilized, and suitable drying agents (i.e. inorganic salts) may be used.

General Materials for Polyol Synthesis from the Fractions of PMTAG

Formic acid (88 wt %) and hydrogen peroxide solution (30 wt %) were purchased from Sigma-Aldrich and perchloride acid (70%) from Fisher Scientific. Hexane, dichloromethane, ethyl acetate and terahydrofuran were purchased from ACP chemical Int. (Montreal, Quebec, Canada) and were used without further treatment.

Synthesis of PMTAG Polyol from the Fractions of PMTAG

PMTAG Polyol was prepared from the solid and liquid fractions of PMTAG in a two-step reaction: epoxidation by formic acid (or acetic acid) and H₂O₂, followed by a hydroxylation using HClO₄ as a catalyst, as described in Scheme 5a when solvent was used and Scheme 5b when solvent was not used. Note that the solvent free procedure was used for the synthesis of polyols from the fractions obtained with the dry fractionation—quiescent method (D2), but not from those obtained with dry fractionation—rates method (D1) or the solvent aided method (S).

### Conventional Method

Standardized polyols were synthesized as described in Scheme 5a using an optimized procedure that has been outlined for PMTAG Polyol.

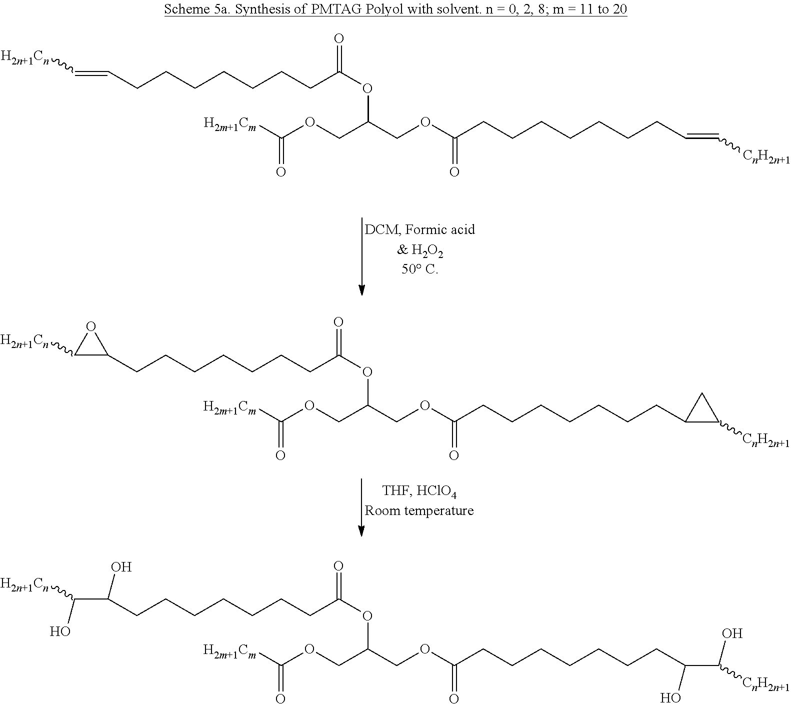

### Epoxidation Procedure

Formic acid (88%; 200 g) was added to a solution of PMTAG (200 g) in dichloromethane (240 mL). This mixture was cooled to 0° C. Hydrogen peroxide (30%, 280 g) was added dropwise. The resulting mixture was stirred at 50° C., and the progress of the reaction was monitored by a combination of TLC and ¹H-NMR. The reaction was completed after 48 to 50 h.

Upon completion, the reaction mixture was diluted with 250 mL dichloromethane, washed with water (200 mL×2), and then with saturated sodium hydrogen carbonate (200 mL×2), and water again (200 mL×2), then dried over anhydrous sodium sulfate. After removing the drying agent by filtration, solvent was removed by rotary evaporation.

### Hydroxylation Procedure

Approximately 200 g crude epoxide was dissolved into a 500 mL solvent mixture of THF: H₂O (3:2) containing 14.5 g perchloric acid. The reaction mixture was stirred at room temperature and the progress of the reaction was monitored by a combination of TLC and ¹H-NMR. The reaction was completed after 36 h. The reaction mixture was poured into 240 mL water and extracted with CH₂Cl₂ (2×240 mL). The organic phase was washed by water (2×240 mL), followed by 5% aqueous NaHCO₃ (2×200 mL) and then water (2×240 mL) again. The organic phase was then dried over Na₂SO₄. After removing the drying agent by filtration, the solvent was removed with a rotary evaporator and further dried by vacuum overnight, giving a light yellow grease-like solid.

### Solvent Free Procedure of Synthesis of PMTAG Polyol

PMTAG Polyol was prepared from the solid and liquid fractions of PMTAG in a two-step reaction: epoxidation by formic acid (or acetic acid) and H₂O₂, followed by a hydroxylation using HClO₄ as a catalyst, as described in Scheme 5b.

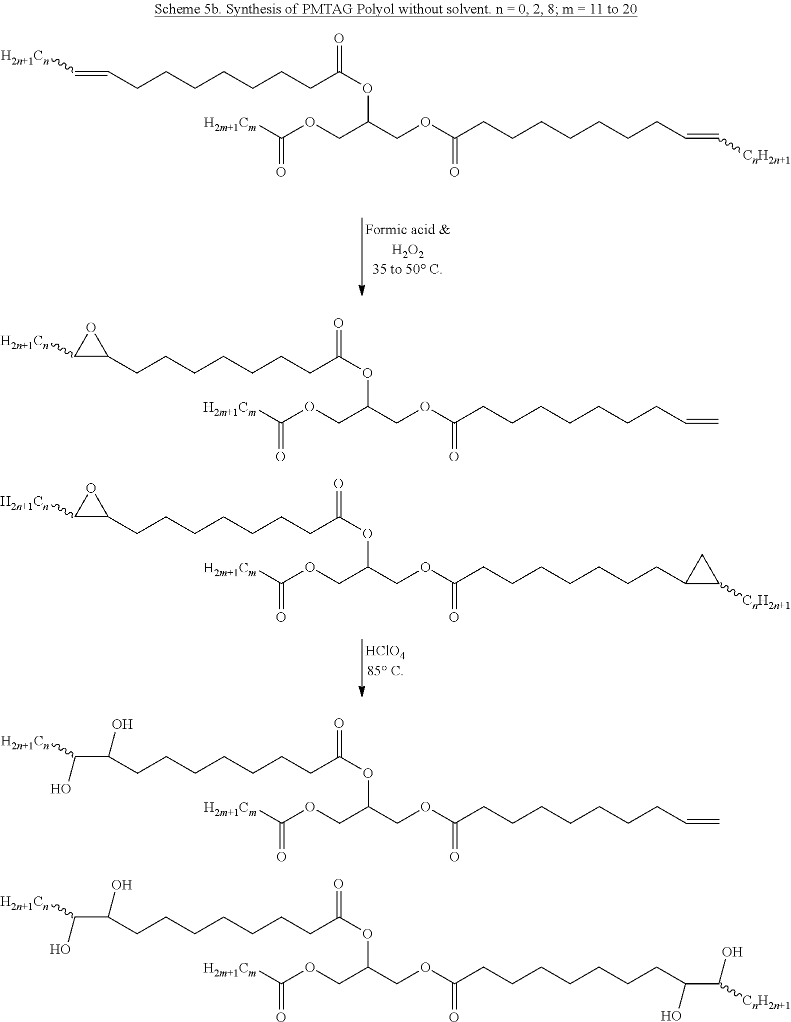

### Epoxidation Procedure

2 kg PMTAG was added into 2 kg formic acid (88%) in a reactor. The temperature was controlled at 30-35° C. 2.8 L of hydrogen peroxide (30%) was added to the reactor slowly (addition rate of ˜1 L/h) with good stirring to maintain the reaction temperature under 50° C. The reaction temperature was raised to ˜48° C. after the hydrogen peroxide was all added. The reaction was continued at 45 to 48° C. overnight, and then the reaction mixture was washed with 1×2 L water, 1×1 L 5% NaHCO₃ and 2×2 L water sequentially. The mixture was used for next step directly.

### Hydroxylation Procedure

The epoxide of PMTAG (2 kg) was added into 10 L water, and then 140 g HClO₄ (70%) was added to the reactor. The reaction mixture was heated to 80-85° C. for 16 h. The reaction was kept still for phase separation. The organic layer was separated from the water layer. The organic layer was washed with 1×2 L water, 1×1 L 5% NaHCO₃ and 2×2 L water sequentially, and then dried on a rotary evaporator.

Analytical Methods for Polyol from the Fractions of PMTAG

The PMTAG Polyols were analyzed using different techniques. These techniques can be broken down into: (i) chemistry characterization techniques, including OH value, acid value, nuclear magnetic resonance (NMR), and high pressure liquid chromatography (HPLC); and (ii) physical characterization methods, including thermogravimetric analysis (TGA), differential scanning calorimetry (DSC), and rheology.

### Chemistry Characterization Techniques for LF(S)- and SF(S)-PMTAG Polyols

OH and acid values of the PMTAG Polyol was determined according to ASTM S957-86 and ASTM D4662-03, respectively.

¹H-NMR spectra were recorded in CDCl₃ on a Varian Unity-INOVA at 499.695 MHz. ¹H chemical shifts are internally referenced to CDCl₃ (7.26 ppm). All spectra were obtained using an 8.6 μs pulse with 4 transients collected in 16 202 points. Datasets were zero-filled to 64 000 points, and a line broadening of 0.4 Hz was applied prior to Fourier transforming the sets. The spectra were processed using ACD Labs NMR Processor, version 12.01.

HPLC analysis was performed on a Waters Alliance (Milford, Mass.) e2695 HPLC system fitted with a Waters ELSD 2424 evaporative light scattering detector. The HPLC system was equipped with an inline degasser, a pump, and an auto-sampler. The ELSD nitrogen flow was set at 25 psi with nebulization and drifting tube maintained at 12° C. and 55° C., respectively. Gain was set at 500. All solvents were HPLC grade and obtained from VWR International, Mississauga, ON. The analysis was performed on a Betasil Diol column (250 mm×4.0 mm, 5.0 m). The temperature of the column was maintained at 50° C. The mobile phase was started with heptane: ethyl acetate (90:10)v run for 1 min at a flow rate of 1 mL/min and in a Gradient mode, then was changed to heptane: ethyl acetate (67:33) in 55 min and then the ratio of Ethyl acetate was increased to 100% in 20 min and held for 10 min. 5 mg/ml (w/v) solution of crude sample in chloroform was filtered through single step filter vial, and 4 μL of sample was passed through the diol column by normal phase in Gradient mode. Waters Empower Version 2 software was used for data collection and data analysis. Purity of eluted samples was determined using the relative peak area.

Physical Characterization Techniques for Polyols from PMTAG Fractions

TGA was carried out on a TGA Q500 (TA Instruments, DE, USA) equipped with a TGA heat exchanger (P/N 953160.901). Approximately 8.0-15.0 mg of sample was loaded in the open TGA platinum pan. The sample was heated from 25 to 600° C. under dry nitrogen at a constant rate of 10° C./min.

DSC measurements of the PMTAG Polyol were run on a Q200 model (TA Instruments, New Castle, Del.) under a nitrogen flow of 50 mL/min. PMTAG Polyol samples between 3.5 and 6.5 (±0.1) mg were run in standard mode in hermetically sealed aluminum DSC pans. The sample was equilibrated at 90° C. for 10 min to erase thermal memory, and then cooled at 5.0° C./min to −90° C. where it was held isothermally for 5 min, and subsequently reheated at a constant rate of 5.0° C./min to 90° C. The “TA Universal Analysis” software was used to analyze the DSC thermograms and extract the peak characteristics. Characteristics of non-resolved peaks were obtained using the first and second derivatives of the differential heat flow.

A temperature-controlled Rheometer (AR2000ex, TA Instruments, DE, USA) was used to measure the viscosity and flow property of the PMTAG Polyol using a 40 mm 2° steel geometry. Temperature control was achieved by a Peltier attachment with an accuracy of 0.1° C. Shear Stress was measured at each temperature by varying the shear rate from 1 to 1200 s⁻¹. Measurements were taken at 10° C. intervals from high temperature (100° C.) to 10° C. below the DSC onset of crystallization temperature of each sample. Viscosities of samples were measured from each sample's melting point up to 110° C. at constant temperature rate (1.0 and 3.0° C./min) with constant shear rate (200 s⁻¹). Data points were collected at intervals of 1° C. The viscosity obtained in this manner was in very good agreement with the measured viscosity using the shear rate/share stress. The shear rate range was optimized for torque (lowest possible is 10 Nm) and velocity (maximum suggested of 40 rad/s).

Results of Synthesis of Polyol from the Solid and Liquid Fractions of PMTAG

### ¹H-NMR Results of Epoxidized LF- and SF-PMTAG

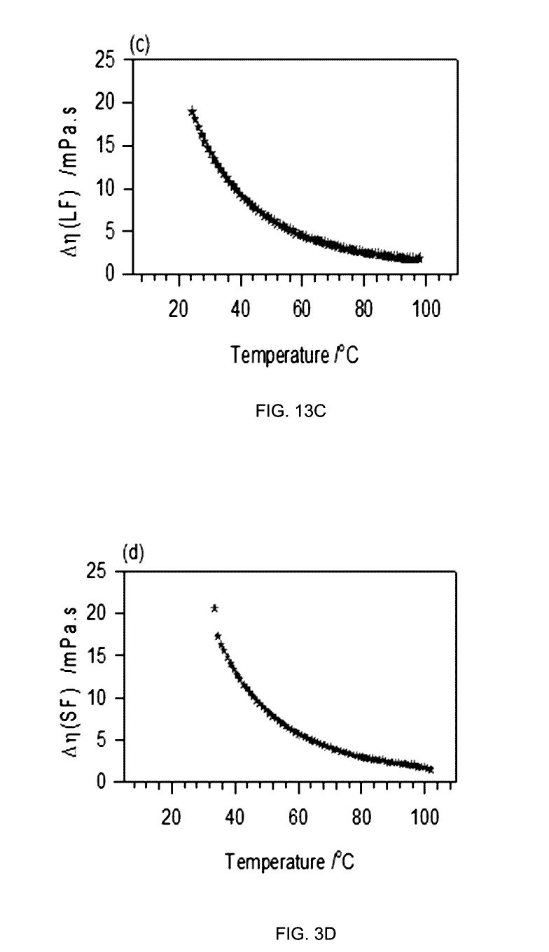

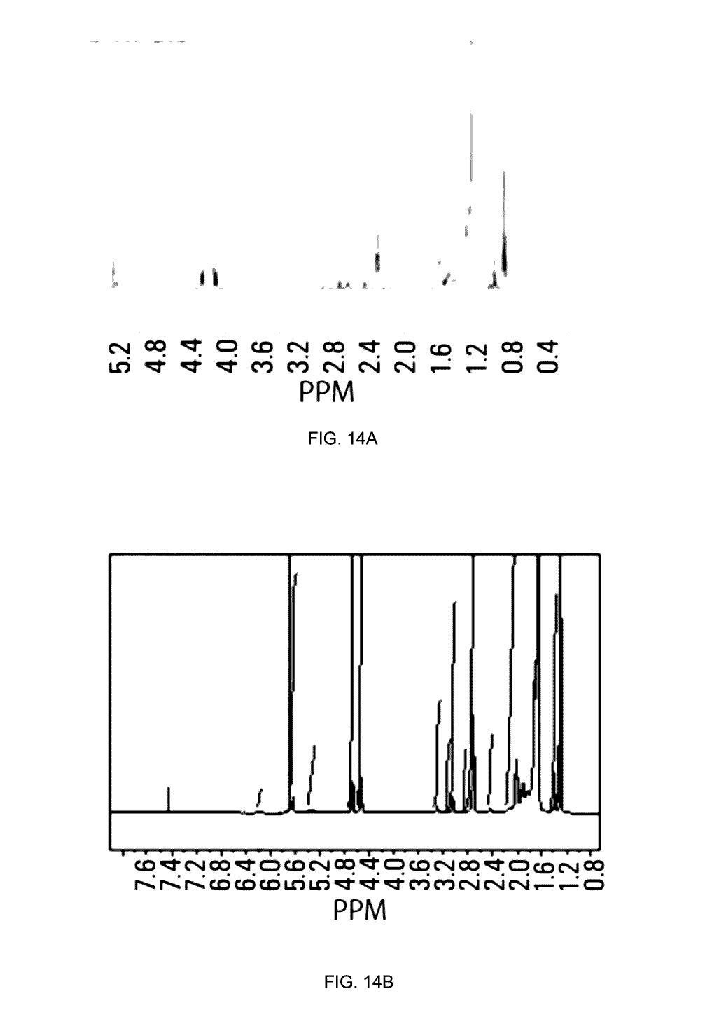

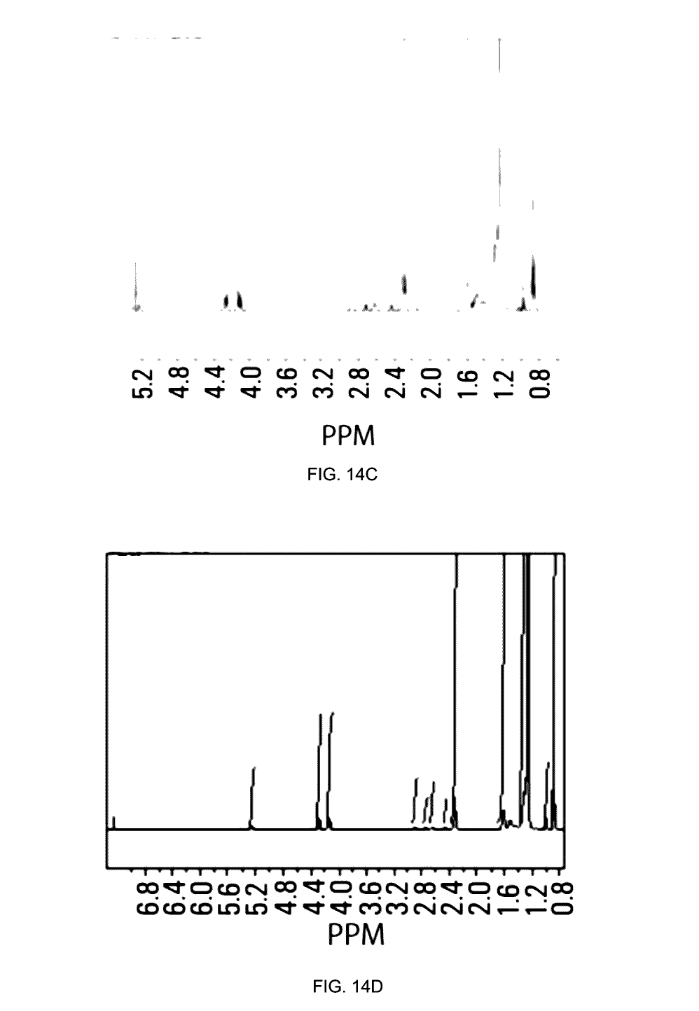

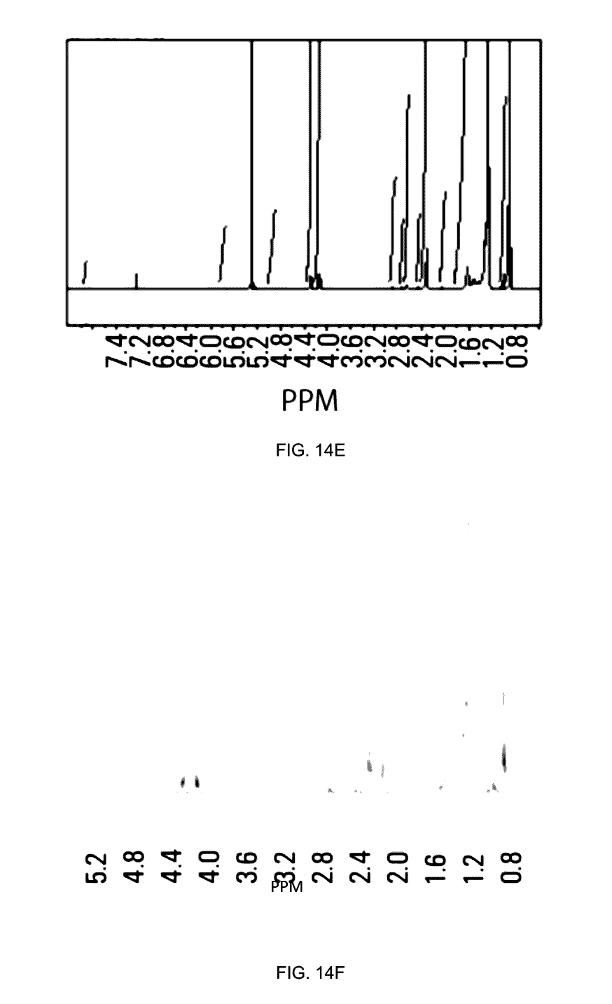

The ¹H-NMR of Epoxy of LF(D1)-PMTAG and SF(D1)-PMTAG (Epoxy LF(D1)-PMTAG and Epoxy SF(D1)-PMTAG, respectively) are shown in FIGS. 14A-14F, respectively.

In all the epoxidized fractions, the protons of the glycerol skeleton, —CH₂CH(O)CH₂— and —OCH₂CHCH₂O— are present at δ 5.3-5.2 ppm and 4.4-4.1 ppm, respectively; —C(═O)CH₂— at δ 2.33-2.28 ppm; α-H to —CH═CH— at δ=2.03-1.98 ppm; and —C(═O)CH₂CH₂— at δ 1.60 ppm. There are two types of —CH₃, one with n=2 and another with n=8. The first presented a proton at δ=1.0-0.9 ppm, and the second a proton at 0.9-0.8 ppm.

In the epoxidized LF(D1)- and SF(D1)-PMTAG and the epoxidized LF(S)- and SF(S)-PMTAG, the chemical shift at 5.8, 5.4 and 5.0 ppm, characteristic of double bonds, disappeared, whereas, the chemical shift at 2.85 ppm, related to non-terminal epoxy ring, and the chemical shift at 2.7 to 2.4 ppm, related to terminal epoxy ring, appeared, indicating that the epoxidation reaction was successful and complete.

In the epoxidized LF(D2)- and SF(D2)-PMTAG, the chemical shift at δ 5.8 ppm and 5.0 to 4.9 ppm are related to the terminal double bond —CH═CH₂ and —CH═CH₂, respectively, which indicate that the epoxidation of the terminal double bonds was not complete. The chemical shift at δ 5.5 ppm to δ 5.3 ppm related to the internal double bond (—CH═CH—) disappeared, indicating that all of the internal double bonds were converted into epoxy rings. The chemical shift at 2.85 ppm that is related to non-terminal epoxy ring, and the chemical shift at 2.7 to 2.4 ppm that is related to terminal epoxy ring, appeared, indicating that the epoxidation reaction was successful.

Results of the Synthesis of Polyols from LF- and SF-PMTAG

Standard polyols were obtained from both the liquid and solid fractions of PMTAG. As listed in Table 15, the produced Polyol presented very low acid values and high OH numbers. Note that standard polyols from the liquid and solid fractions obtained by dry quiescent fractionation of PMTAG (LF(D2)-PMTAG Polyol and SF(D2)-PMTAG Polyol, respectively) were synthesized without solvent.

TABLE 15
Acid value and OH number of PMTAG polyols

| | | |
|---|---|---|
| | OH Value | Acid Value |
| Iodine Value | (mg KOH/g) | (mg KOH/g) |

Polyols from Liquid Fractions

| | | | |
|---|---|---|---|
| LF(D1)-PMTAG Polyol | — | 184 | <4 |
| LF(D2)-PMTAG Polyol | 9 | 170 | <2.3 |
| LF(S)-PMTAG Polyol | — | 182 | <4 |

Polyols from Solid Fractions

| | | | |
|---|---|---|---|
| SF(D1)-PMTAG Polyol | — | 136 | <3 |
| SF(D2)-PMTAG Polyol | 5 | 80 | <1.3 |
| SF(S)-PMTAG Polyol | — | 136 | <3 |

### Compositional Analysis of SF- and LF-PMTAG Polyols

The theoretical structures of SF- and LF-PMTAG Polyols based on the TAG analysis of palm oil are given below in Scheme 6. The actual composition of the PMTAG Polyols was characterized by ¹H-NMR and HPLC.

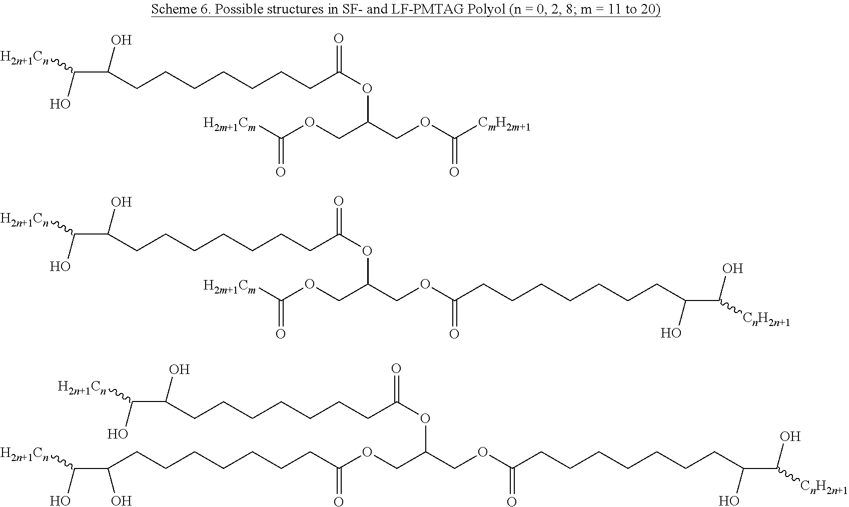

### ¹H-NMR Results of Standard LF- and SF-PMTAG Polyols

¹H-NMR Results of Standard LF-PMTAG Polyols

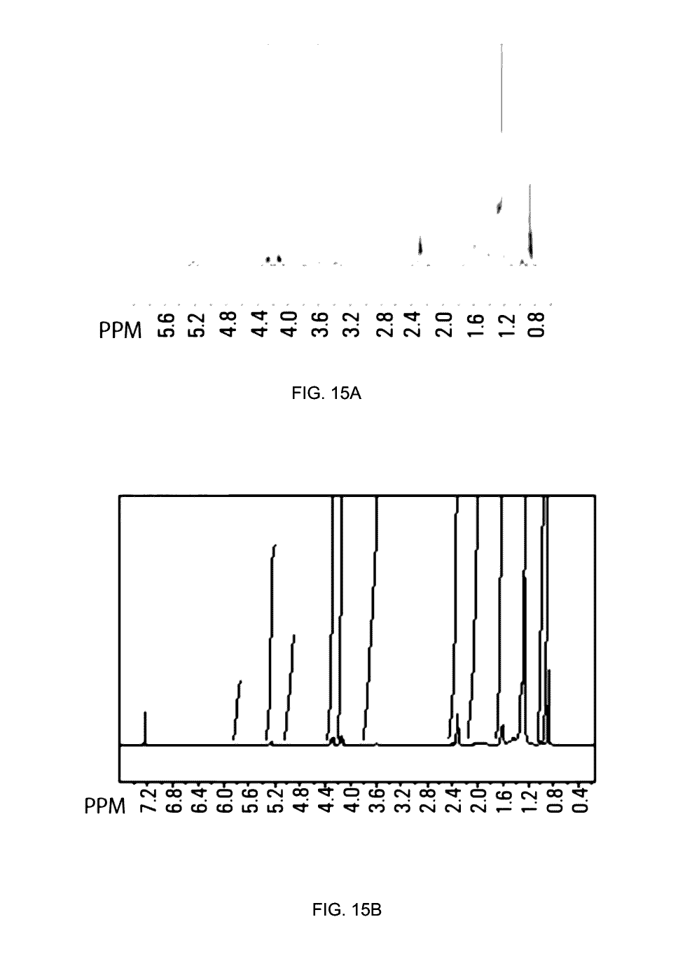

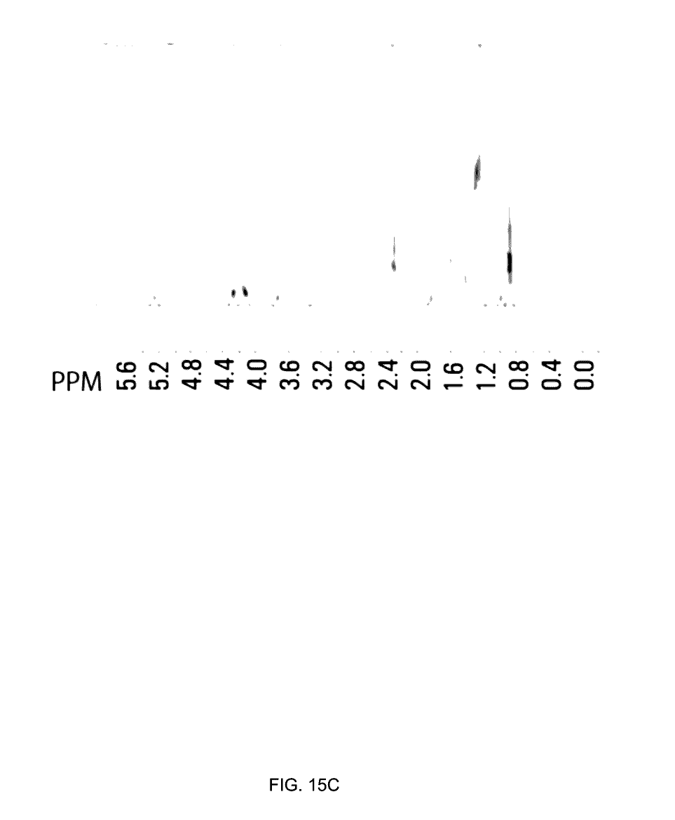

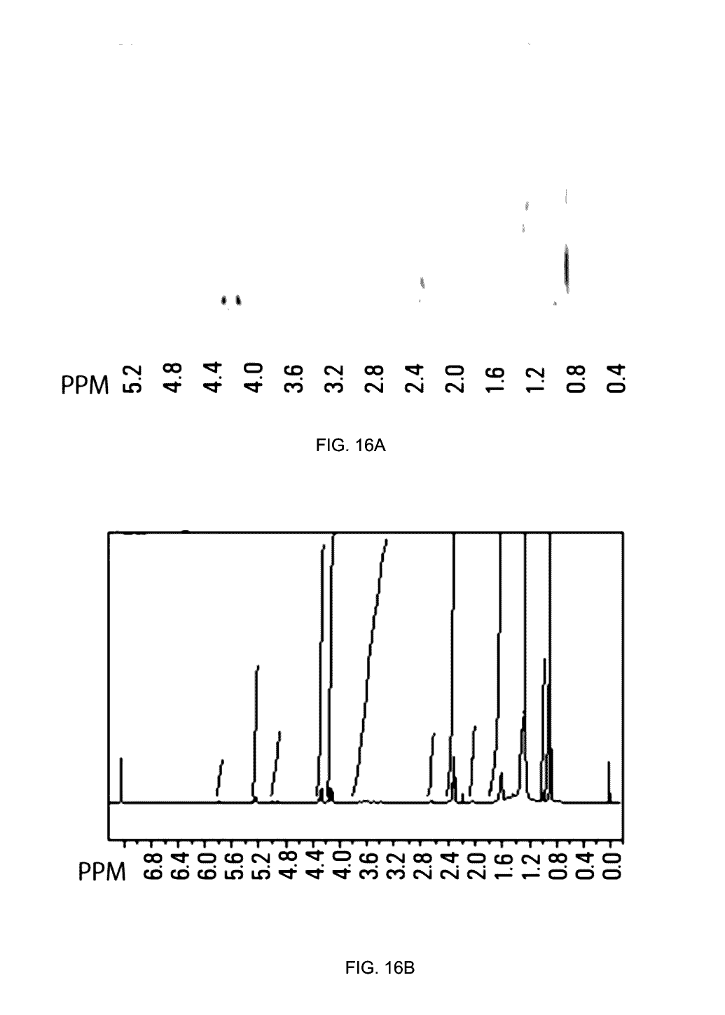

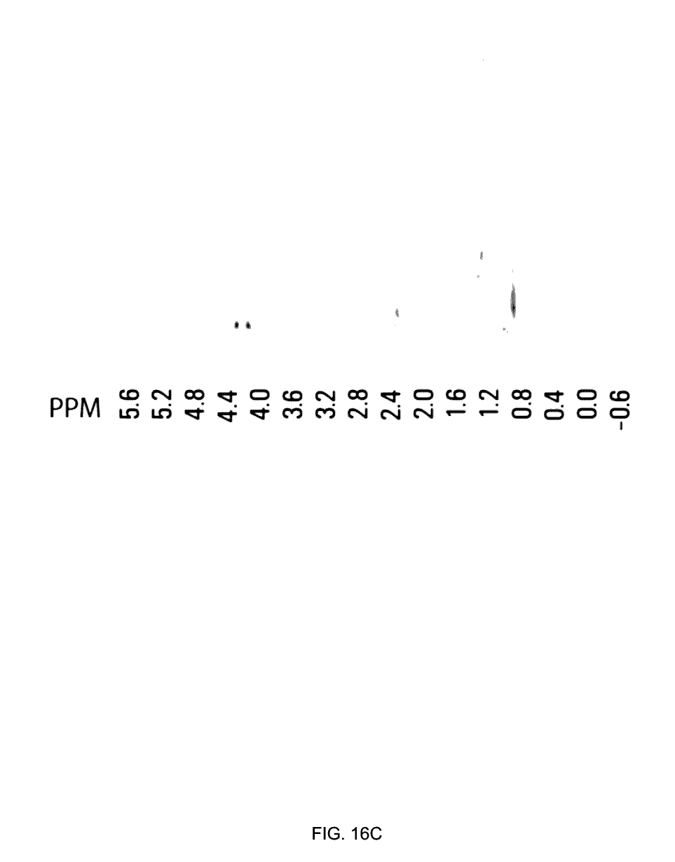

The ¹H-NMR of polyols obtained by the different fractionation methods—Dry rates method (D1), Dry quiescent method (D2) and Solvent aided method (S) are shown in FIGS. 15A, 15B, and 15C, respectively, for the liquid fractions, and in FIGS. 16A, 16B, and 16C, respectively, for the solid fractions. The related ¹H-NMR chemical shifts, δ, in CDCl₃ are listed in Table 16.

The spectra of all polyols presented the chemical shifts at 3.8-3.4 ppm related to protons neighbored by —OH and did not present the chemical shifts at 2.8-2.4 ppm related to epoxy ring, indicating that the hydroxylation of the epoxy ring was complete. A typical TAG-like glycerol backbone was clearly shown in the ¹H-NMR spectra of all the polyols, indicating that the hydrolysis of the ester link in TAG was avoided.

TABLE 16

¹H-NMR Chemical shifts, δ, in CDCl₃ (ppm)

Liquid Fractions

| | |
|---|---|
| LF(D1)-PMTAG Polyol | 5.2 (D2), 4.4-4.2 (dd), 4.2-4.0 (dd), 3.8-3.2 |
| | (D2), 2.4-2.2 (D2), 1.6-1.2 (D2), 1.0 (t), 0.8 (t) |
| LF(D2)-PMTAG Polyol | 5.8 (D2), 5.2 (D2), 5.0-4.8 (dd), 4.4-4.2 (dd), |
| | 4.2-4.0 (dd), 3.8-3.2 (D2), 2.4-2.2 (D2), |
| | 1.6-1.2 (D2), 1.0 (t), 0.8 (t) |
| LF(S)-PMTAG Polyol | 5.2 (D2), 4.4-4.2 (dd), 4.2-4.0 (dd), 3.8-3.2 |
| | (D2), 2.4-2.2 (D2), 1.6-1.2 (D2), 1.0 (t), 0.8 (t) |

Solid Fractions

| | |
|---|---|
| SF(D1)-PMTAG Polyol | 5.2 (D2), 4.4-4.2 (dd), 4.2-4.0 (dd), 3.8-3.2 |
| | (D2), 2.4-2.2 (D2), 1.6-1.2 (D2), 1.0 (t), 0.8 (t) |
| SF(D2)-PMTAG Polyol | 5.8 (D2), 5.2 (D2), 5.0-4.8 (dd), 4.4-4.2 (dd), |
| | 4.2-4.0 (dd), 3.8-3.2 (D2), 2.4-2.2 (D2), |
| | 1.6-1.2 (D2), 1.0 (t), 0.8 (t) |
| SF(S)-PMTAG Polyol | 5.2 (D2), 4.4-4.2 (dd), 4.2-4.0 (dd), 3.8-3.2 |
| | (D2), 2.4-2.2 (D2), 1.6-1.2 (D2), 1.0 (t), 0.8 (t) |

### HPLC of LF- and SF-PMTAG Polyol Results

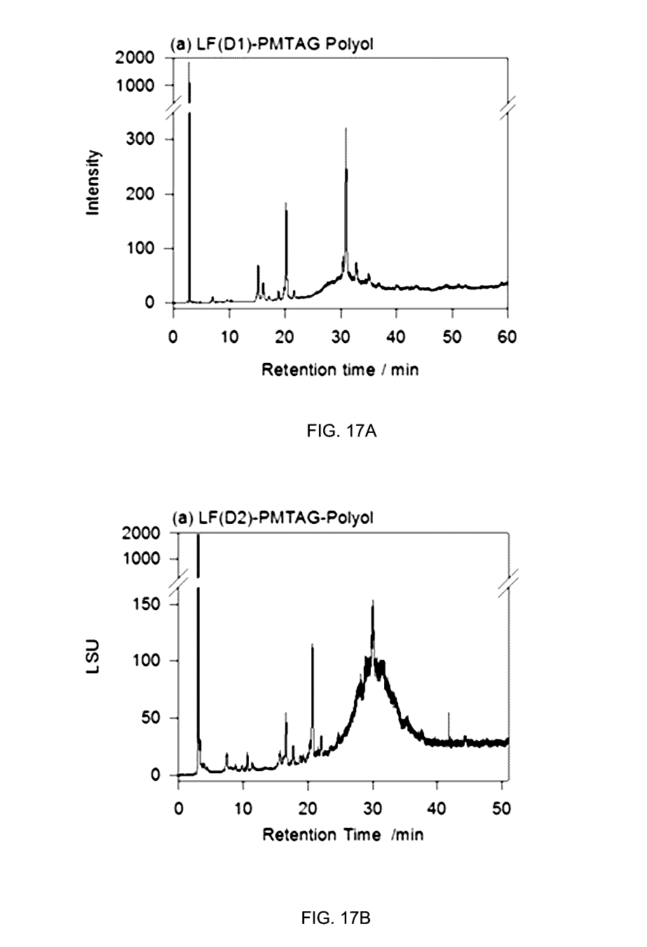

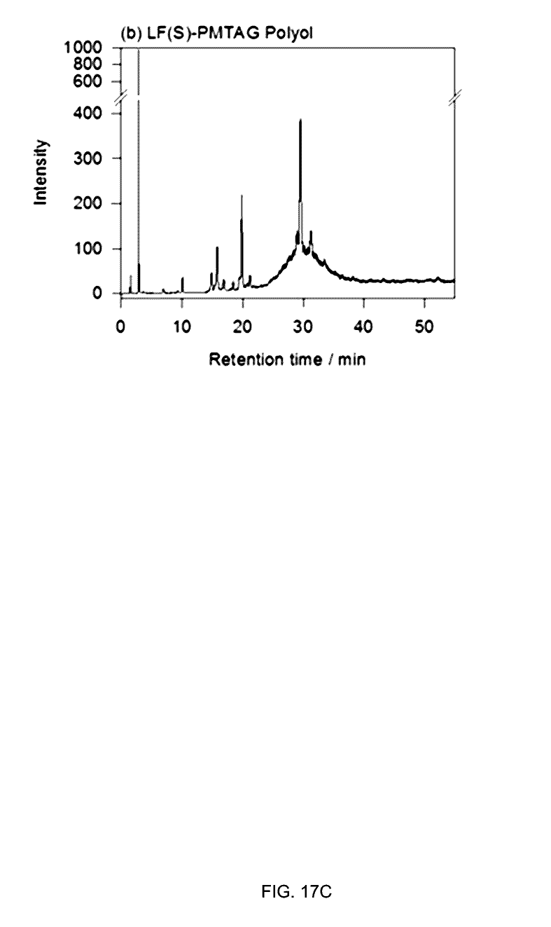

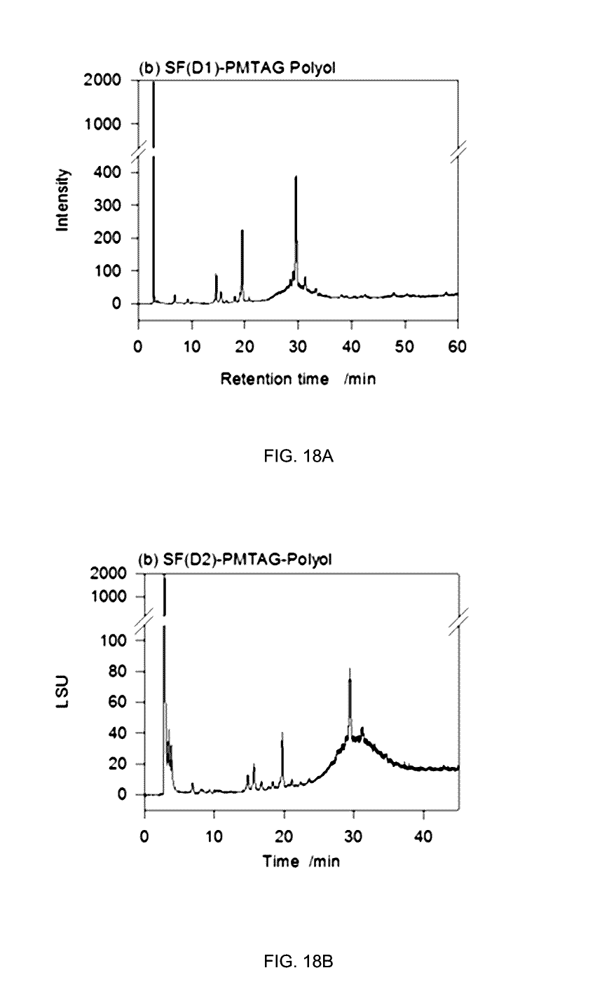

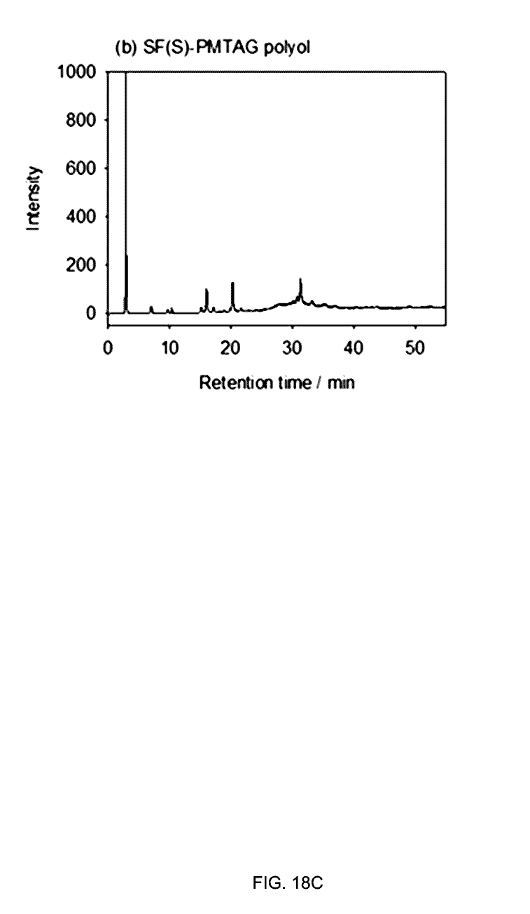

The HPLC curve of the Polyols obtained from PMTAG with the dry fractionation rates method (D1), dry fractionation quiescent method (D2), and solvent fractionation method (S), are shown in FIGS. 17A, 17B, and 17C, respectively, for the liquid fractions, and in FIGS. 18A, 18B, and 18C, respectively, for the solid fractions. HPLC results and analyses are listed in Table 17. HPLC of the polyol obtained from non-fractionated PMTAG obtained via the conventional route (PMTAG Polyol) and the green route (PMTAG Green Polyol) are shown in FIGS. 19A and 19B for comparison purposes. Corresponding data are listed in Table 18.

TABLE 17a
HPLC Retention Time (RT, min) of LF(D1)-,
LF(D2)- and SF(D1)-PMTAG Polyols

| | LF(D1)-PMTAG Polyol | | LF(D2)-PMTAG Polyol | | LF(S)-PMTAG Polyol | |
|---|---|---|---|---|---|---|
| | RT | Area | RT | Area | RT | Area |
| Peak | (min) | (%) | (min) | (%) | (min) | (%) |
| 1 | 2.38 | 0.11 | 2.9 | 67.96 | 1.52 | 0.37 |
| 2 | 2.82 | 20.71 | 7.4 | 1.61 | 2.84 | 5.76 |
| 3 | 7.01 | 0.52 | 8.8 | 0.42 | 7.01 | 0.28 |
| 4 | 10.26 | 0.36 | 9.8 | 0.44 | 10.09 | 0.59 |
| 5 | 15.16 | 3.57 | 10.6 | 1.38 | 14.89 | 1.23 |
| 6 | 16.05 | 2.39 | 15.7 | 1.76 | 15.82 | 2.97 |
| 7 | 17.15 | 0.62 | 16.6 | 5.05 | 16.89 | 0.95 |
| 8 | 18.81 | 0.88 | 17.7 | 1.81 | 18.47 | 0.85 |
| 9 | 19.86 | 0.84 | 18.8 | 0.62 | 19.87 | 5.99 |
| 10 | 20.19 | 8.71 | 19.3 | 0.51 | 21.2 | 1.49 |
| 11 | 21.56 | 0.91 | 20.3 | 1.73 | 25.83 | 5.34 |
| 12 | 26.65 | 1.33 | 20.7 | 8.82 | 27.82 | 11.10 |
| 13 | 27.09 | 1.42 | 22.0 | 1.15 | 28.44 | 4.48 |
| 14 | 27.56 | 1.54 | 30.0 | 6.74 | 28.98 | 5.17 |
| 15 | 28.40 | 4.82 | | | 29.55 | 14.91 |
| 16 | 29.90 | 5.85 | | | 30.21 | 7.80 |
| 17 | 30.38 | 5.65 | | | 31.24 | 7.00 |
| 18 | 30.91 | 20.47 | | | 31.90 | 8.80 |
| 19 | 31.55 | 6.06 | | | 33.49 | 7.03 |
| 20 | 32.73 | 6.75 | | | 35.31 | 7.89 |
| 21 | 34.90 | 4.97 | | | | |
| 22 | 39.93 | 1.52 | | | | |

TABLE 17b
HPLC data of SF(D1)-PMTAG Polyol, SF(D2)-PMTAG Polyol
and SF(S)-PMTAG Polyol. RT: Retention Time (min)

| | SF(D1)-PMTAG Polyol | | SF(D2)-PMTAG Polyol | | SF(S)-PMTAG Polyol | |
|---|---|---|---|---|---|---|
| | RT | Area | RT | Area | RT | Area |
| Peak | (min) | (%) | (min) | (%) | (min) | (%) |
| 1 | 2.78 | 31.37 | 2.8 | 81.22 | 2.86 | 38.29 |
| 2 | 6.86 | 0.79 | 3.5 | 3.61 | 7.04 | 1.27 |
| 3 | 9.28 | 0.65 | 3.9 | 2.28 | 9.66 | 0.82 |
| 4 | 14.60 | 3.18 | 6.9 | 0.57 | 10.30 | 0.57 |
| 5 | 15.42 | 1.91 | 8.1 | 0.84 | 15.13 | 1.09 |
| 6 | 18.11 | 0.77 | 14.8 | 1.09 | 15.97 | 4.30 |
| 7 | 19.47 | 7.56 | 15.7 | 2.14 | 17.11 | 1.26 |
| 8 | 20.79 | 0.61 | 18.4 | 0.41 | 18.87 | 0.59 |
| 9 | 28.42 | 15.04 | 19.8 | 2.72 | 20.23 | 5.72 |
| 10 | 29.01 | 3.66 | 21.1 | 0.27 | 21.61 | 0.98 |
| 11 | 29.57 | 16.06 | 23.5 | 0.24 | 22.90 | 0.40 |
| 12 | | | 27.6 | 0.54 | 27.78 | 11.24 |
| 13 | 29.95 | 4.91 | 29.4 | 3.29 | 30.70 | 9.68 |
| 14 | | | 31.2 | 0.79 | 31.29 | 8.07 |
| 15 | 31.29 | 6.07 | | | 32.01 | 3.47 |
| | 33.27 | 3.56 | | | 33.15 | 3.62 |
| | 34.03 | 3.03 | | | 33.78 | 3.60 |
| | | | | | 35.29 | 2.41 |
| | | | | | 36.88 | 2.61 |
| | 38.18 | 0.51 | | | | |
| | 39.00 | 0.32 | | | | |
| 16 | | | | | | |

TABLE 18
HPLC of PMTAG Polyol and PMTAG Green Polyol

| | |
|---|---|
| PMTAG Polyol | PMTAG Green Polyol |

| | RT | Area | RT | Area |
|---|---|---|---|---|
| Peak | (min) | (%) | (min) | (%) |
| 1 | 2.4 | 0.90 | 2.8 | 72.15 |
| 2 | 2.8 | 42.17 | 3.1 | 1.16 |
| 3 | 7.2 | 0.67 | 6.9 | 0.70 |
| 4 | 9.8 | 0.18 | 10.0 | 0.58 |
| 5 | 10.5 | 0.08 | 14.8 | 1.16 |
| 6 | 15.7 | 5.07 | 15.7 | 4.04 |
| 7 | 16.6 | 1.66 | 16.7 | 1.21 |
| 8 | 17.6 | 0.30 | 18.3 | 1.20 |
| 9 | 19.4 | 0.94 | 19.7 | 9.64 |
| 10 | 20.9 | 14.04 | | |
| 11 | 22.3 | 0.95 | | |
| 12 | 30.8 | 3.05 | 29.4 | 6.84 |
| 13 | 31.4 | 25.58 | 31.2 | 1.32 |
| 14 | 33.2 | 2.99 | | |
| 15 | 35.6 | 0.96 | | |
| 16 | 40.4 | 0.45 | | |

### Structures of LF- and SF-PMTAG Polvols

The analysis of the HPLC of the different PMTAG Polyols was carried out with the help of PMTAG Polyol fractions separated using column chromatography (Table 19).

The structures of LF- and SF-PMTAG Polyols suggested based on HPLC and ¹H-NMR are shown in Scheme 7. These structures can be directly related to the theoretical structures of PMTAG Polyols based on the TAG composition of PMTAG shown in Scheme 6. The saturated TAG composition appeared at 2.80 min; the hydrolyzed by-products at 7 to 12 min; PMTAG diols with long fatty acid chain at 15 to 19 min; PMTAG diols with short fatty acid chain, or PMTAG tetrols with long fatty acid chain at 19 to 21 min; PMTAG tetrols with short fatty acid and PMTAG diols with terminal OH group at 21 to 23 min; PMTAG tetrols with terminal OH group and PMTAG hexols appeared at 30 min and up.

The HPLC results indicate that the polyols produced from the different fractions are composed of the same fractions, but with a different content for each fraction. More fractions eluting at ˜19 to 29.5 min were presented in SF-PMTAG Polyol, and more fractions eluting at 29.5 min and up were presented in SF-PMTAG Polyol. There are more saturated TAGs (RT=2.8 min), long chain diols, tetrols and hexols (RT=17 to 28 min), but less short chain tetrols and hexols (RT>29 min) in SF-PMTAG Polyol than in LF-PMTAG Polyol. There are less diols (long and short fatty acid chains), diols with terminal OH group, and tetrols with long fatty acid chain in LF-PMTAG polyol than in LF-PMTAG polyol.

TABLE 19
Characterization of PMTAG polyol fractions obtained from column chromatography.
RT: HPLC Retention Time. Structures: suggested based on ¹H-NMR and MS (Scheme 4).

| | RT | Area | MS and possible | |
|---|---|---|---|---|
| Fraction | (min) | (%) | formula | Structures |
| F1 | 2.801 | 42.2 | 947.8 (C₆₁H₁₁₈O₆) | Saturated TAGs |
| | | | 849.8 (C₅₄H₁₀₄O₆) | |
| F2 | 7.196 | 0.7 | 667.5 (C₄₂H₈₂O₅) | Not a TAG structure; |
| | | | | Contain hydrolysed by-products |
| F3 | 9.827 | 0.2 | — | Mixture of F1, F2, and unreacted |
| | | | | terminal double bond structures |
| F4 | 10.531 | 0.1 | 825.29 (C₅₀H₉₆O₈) | Not typical TAG structure; |
| | | | | Contain hydrolysed by-products |
| | | | | with oleic acid derived diols |
| F5 | 15.660 | 5.1 | 884.6 (C₅₃H₁₀₂O₈•H₂O) | TAG-like diols containing one |
| F6 | 16.577 | 1.7 | 889.5 (C₅₂H₁₀₀O₈•2H₂O) | oleic acid-like derived diol |
| F7 | 19.415 | 0.9 | 889.7 (C₅₅H₁₀₆O₈) | TAG-like diols containing one |
| | | | 805.2 (C₄₈H₉₂O₈•H₂O) | oleic acid-like derived or/and one |
| | | | 833.4 (C₄₈H₉₂O₈•2H₂O) | 9-dodenonic acid-like derived |
| | | | | diol |
| F8 | 20.854 | 14.0 | 872.8 (C₅₁H₉₈O₁₀) | TAG-like diols containing one 9- |
| | | | 833.4 (C₄₇H₉₀O₁₀•H₂O, | dodenonic acid-like derived diol; |
| | | | C₄₅H₈₆O₁₀•2H₂O, | TAG-like tetrols containing one |
| | | | C₄₈H₉₂O₈•2H₂O) | or two oleic acid-like derived |
| | | | 805.4 (C₄₅H₈₆O₁₀•H₂O, | diols or/and one 9-dodenonic |
| | | | C₄₈H₉₂O₈•H₂O) | acid-like derived diol |
| F9 | 20.601 | 1.0 | 805.4 (C₄₅H₈₆O₁₀•H₂O) | |
| | 21.945 | | 817.8 (C₄₇H₉₀O₁₀) | |
| | | | 844.8 (C₄₉H₉₄O₁₀) | |
| F10 | 22.296 | 1.0 | 719.5 (C₃₉H₇₄O₁₀•H₂O) | TAGs-like diols containing one9- |
| | | | 805.6 (C₄₅H₈₆O₁₀•H₂O) | denonic acid- like derived diol; |
| | | | 847.6 (C₄₈H₉₂O₁₀•H₂O) | TAGs-like tetrols containing one |
| | | | | or two oleic acid-like, one or two |
| | | | | 9-dodenonic acid-like or/and one |
| | | | | 9-denonic acid- like derived diols |
| F11 | 30.751, | 25.6% + | 777.3 (C₄₂H₈₀O₁₂) | TAG-like hexols containing one |
| | 31.374 | 3.1% | 805.3 (C₄₄H₈₄O₁₂, | or two oleic acid-like and one or |
| | | | C₄₅H₈₆O₁₀•H₂O, | two 9-dodenonic acid-like |
| | | | C₄₈H₉₂O₈•H₂O) | derived diol; |
| | | | 877.7 (C₄₉H₉₄O₁₂) | TAG-like tetrols containing one |
| | | | 651.4 (C₃₃H₆₂O₁₂) | 9-denonic acid- like derivatives |
| | | | | and one oleic acid-like or 9- |
| | | | | dodenonic acid-like derived diol; |
| | | | | TAG-like diols containing one 9- |
| | | | | denonic acid- like derived diol. |

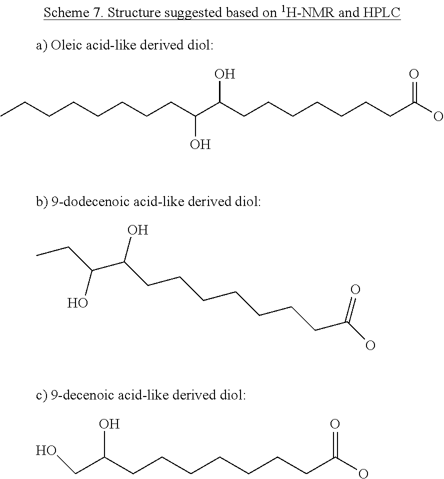 [SMILES: CCCCCCCCC(O)C(O)CCCCCCCC(=O)O ; CCC(O)C(O)CCCCCCCC(=O)O ; O=C(O)CCCCCCCC(O)CO]

### Physical Properties of LF-PMTAG Polyols

### Thermogravimetric Analysis of LF- and SF-PMTAG Polyols

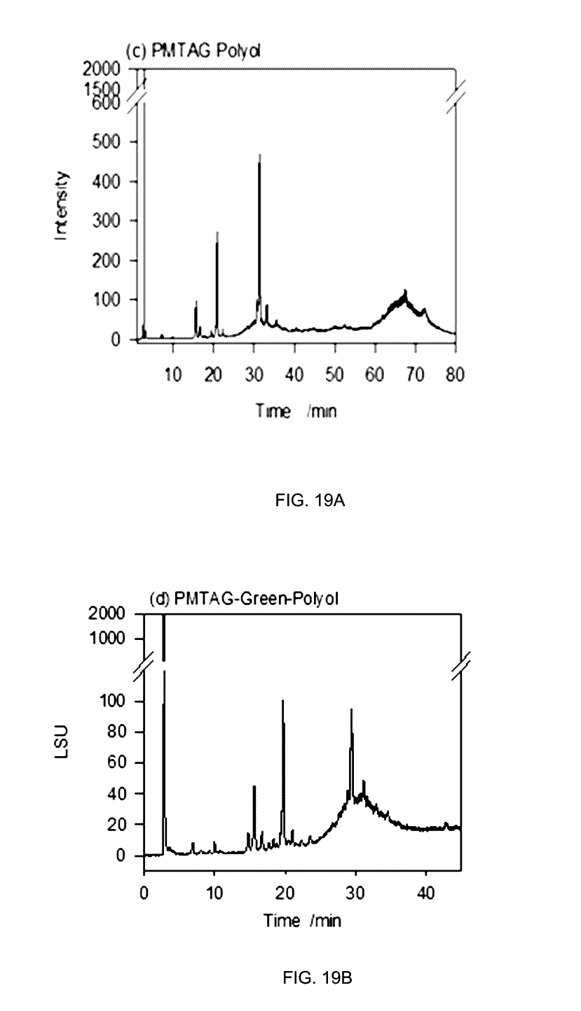

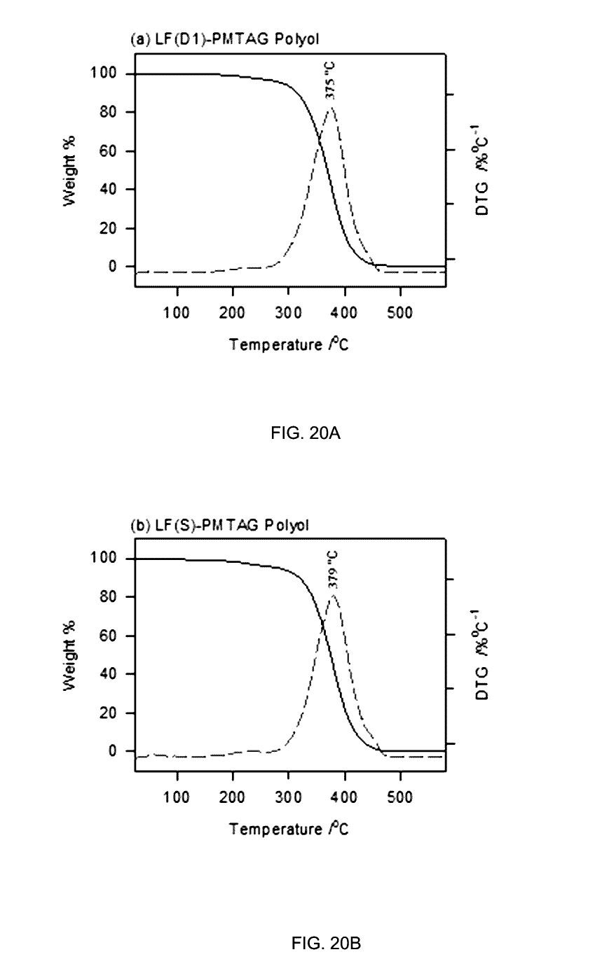

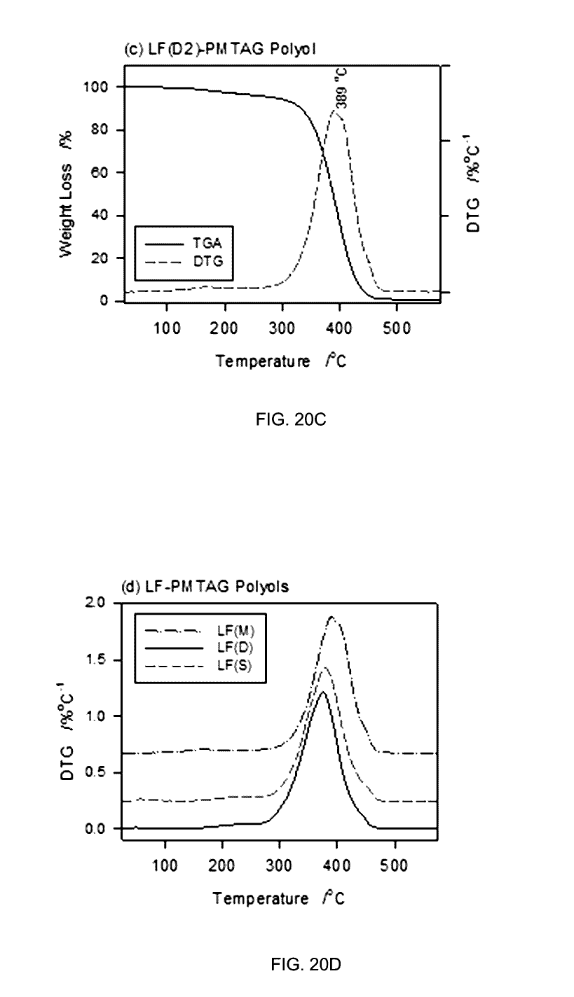

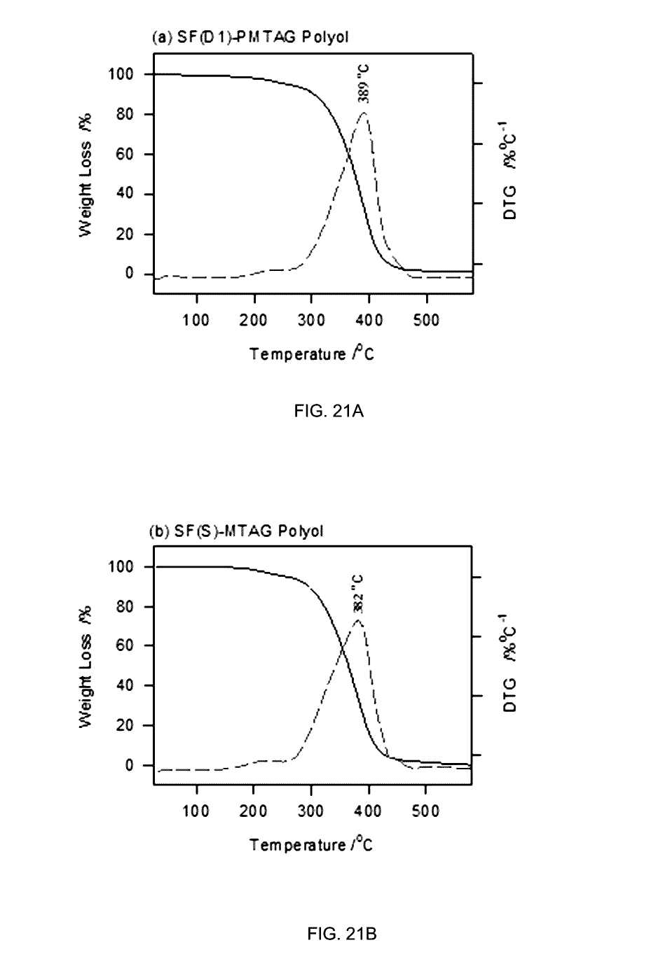

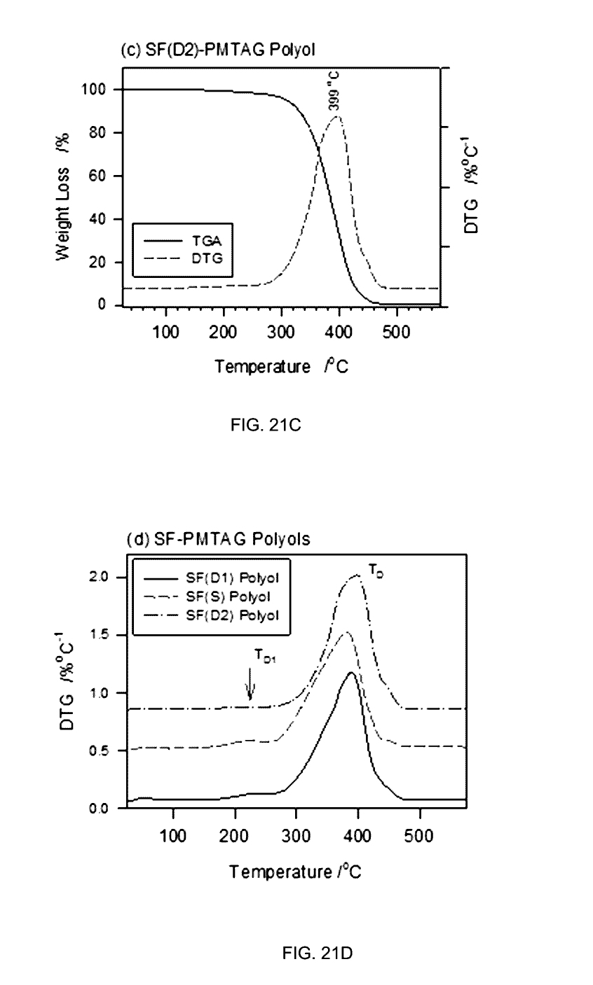

The TGA and DTG profiles of LF(D1)-, LF(S)- and LF(D2)-PMTAG Polyols are shown in FIGS. 20A, 20B, and 20C, respectively, and those of SF(D1)-, SF(S)- and SF(D2)-PMTAG Polyols in FIGS. 21A, 21B, and 21C, respectively. The corresponding data (extrapolated onset and offset temperatures of degradation, temperature of degradation measured at 1, 5 and 10% decomposition, and the DTG peak temperatures) are provided in Table 20. For comparison purposes, the DTG curves of the polyols made from the liquid fractions are presented in FIG. 20D, and those of the solid fraction in FIG. 21D.

The TGA and DTG data indicate that polyols synthesized from the fractions undergo degradation mechanisms similar to the polyols made from the MTAG itself. The DTG curves presented a very weak peak at ˜170 to 240° C. followed by a large peak at 375-400° C. (T_{D1} and T_{D}, respectively, in FIGS. 20 and 21) indicating two steps of degradation. The first step involved ˜1 to 3% weight loss only. The second DTG peak (where ˜50-67% weight loss was recorded), is associated with the breakage of the ester bonds, the dominant mechanism of degradation that was also observed in the TGA of the LF- and SF-PMTAG starting materials.

LF-PMTAG Polyols presented very similar thermal stabilities with practically similar rates of decomposition (˜1.2%°/C. at the DTG peak temperatures); whereas, the SF-PMTAG Polyols thermal stability were somehow different. SF(D2)-PMTAG Polyol was the most stable, followed by SF(D1)- and SF(S)-PMTAG Polyols. The maximum rates of degradation of SF(D2)-PMTAG, SF(D1)-PMTAG and SF(S)-PMTAG Polyols, as recorded at the DTG peaks, were 1.16, ˜1.11 and 1.00%°/C., respectively. Note that all the LF-PMTAG Polyols presented relatively lower thermal stabilities than the SF-PMTAG Polyols. For example, the main degradation step of SF(D2)-PMTAG Polyol peaked 10° C. higher than that of LF(D2)-PMTAG Polyol, and the main DTG peaks of SF(D1)-PMTAG Polyol was 13° C. higher than that of LF(D1)-MTAG Polyol.

TABLE 20
Temperature of degradation at 1, 5 and 10% weight loss (T_{1%}^{d}, T_{5%}^{d},
T_{10%}^{d}, respectively), DTG peak temperatures (T_{D}), and extrapolated onset (Tₒₙ) and
offset (T_{off}) temperatures of degradation of LF- and SF-PMTAG Polyols

| Temperature (° C.) | | | | | | | Weight loss (%) at | | |
|---|---|---|---|---|---|---|---|---|---|
| T_{1%}^{d} | T_{5%}^{d} | T_{10%}^{d} | Tₒₙ | T_{D1} | T_{D} | T_{off} | Tₒₙ | T_{D1} | T_{D} |

Polyols from the liquid fractions of PMTAG

| | | | | | | | | | | |
|---|---|---|---|---|---|---|---|---|---|---|
| LF(D1) | 194 | 291 | 315 | 328 | 228 | 376 | 469 | 15 | 2 | 58 |
| LF(S) | 155 | 288 | 318 | 334 | 232 | 379 | 470 | 15 | 3 | 55 |
| LF(D2) | 153 | 287 | 332 | 344 | 168 | 389 | 431 | 15 | 1.5 | 49 |

Polyols from the solid fractions of PMTAG

| | | | | | | | | | | |
|---|---|---|---|---|---|---|---|---|---|---|
| SF(D1) | 177 | 261 | 304 | 294 | 237 | 389 | 422 | 8 | 4 | 67 |
| SF(S) | 186 | 255 | 296 | 279 | 223 | 382 | 420 | 7 | 3 | 63 |
| SF(D2) | 218 | 310 | 331 | 338 | 215 | 399 | 428 | 12 | 1 | 66 |

### Crystallization and Melting Behavior of LF- and SF-PMTAG Polyols

### Crystallization and Melting Behavior of LF-PMTAG Polyols

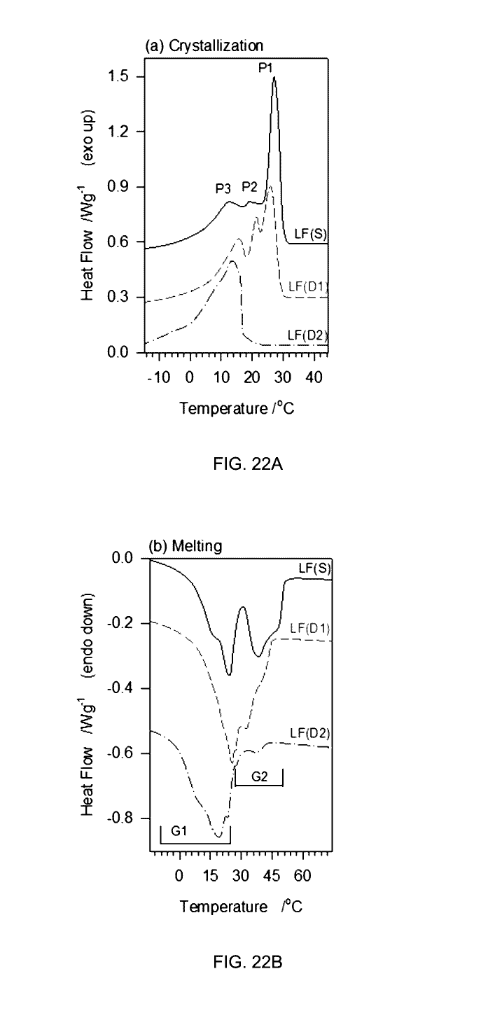

The crystallization and heating profiles (both at 5° C./min) of LF-PMTAG Polyols are shown in FIGS. 22A and 22B, respectively. The corresponding thermal data are listed in Table 21.

LF(S)- and LF(D1)-PMTAG Polyols were liquid above sub ambient temperature (Tₒₙ˜30° C.); whereas, LF(D2)-PMTAG Polyol was liquid at ambient temperature (Tₒₙ˜17° C.). Three defined peaks were observed in the cooling thermograms of LF(S)- and LF(D1)-PMTAG Polyols (P1, P2 and P3 in FIG. 22A) and one peak in LF(D2)-PMTAG Polyol (P3 in FIG. 22A). The presence of P1 and P2 in the cooling thermograms of LF(S)- and LF(D1)-PMTAG Polyols indicates that they contain high melting temperature components that were not present in LF(D2)-PMTAG Polyol. P1 and P2 are therefore collectively associated with a high crystallizing portion of the LF-PMTAG Polyol and the following P3 is associated with its low crystallizing portion.

The heating thermogram of LF(S)- and LF(D1)-PMTAG Polyols displayed two corresponding groups of endothermic events (G1 and G2 in FIG. 22B), constituted of a prominent and shoulder peaks. LF(D2)-PMTAG Polyol presented only G1. G1 and G2 are associated with the melting of the low and high melting portion of the polyols, respectively. Note that the heating thermograms of the LF-PMTAG Polyols did not display any exotherm, suggesting that polymorphic transformations mediated by melt do not occur with the LF-PMTAG Polyols.

TABLE 21
Thermal data of LF-PMTAG Polyols obtained on cooling
and heating (both at 5° C./min). Onset (Tₒₙ), offset (T_{off}),
and peak temperatures (T₁₋₃), Enthalpy of crystallization (Δ
H_{C}), and Enthalpy of melting (ΔH_{M}).

| | | | | | | | Enthalpy |
|---|---|---|---|---|---|---|---|
| | Temperature (° C.) | | | | | | (J/g) |
| Cooling | | | | | | | |
| | Tₒₙ | T₁ | T₂ | T₃ | | T_{off} | ΔH_{C} |
| LF(D1)- | 28.99 | 25.77 | 21.25 | 15.51 | | 0.80 | 99.64 |
| PMTAG | | | | | | | |
| Polyol | | | | | | | |
| LF(S)- | 29.82 | 27.02 | 18.91 | 12.57 | | −0.61 | 93.8 |
| PMTAG | | | | | | | |
| Polyol | | | | | | | |
| LF(D2)- | 16.87 | — | — | 16.87 | | −37.72 | 84.1 |
| PMTAG | | | | | | | |
| Polyol | | | | | | | |
| Heating | | | | | | | |
| | Tₒₙ | T₁^{a} | T₂ | T₃ | T₄^{a} | T_{off} | ΔH_{M} |
| LF(D1)- | 6.60 | 41.58 | 32.48 | 27.23 | 20.88 | 44.80 | 89.9 |
| PMTAG | | | | | | | |
| Polyol | | | | | | | |
| LF(S)- | 3.74 | 48.35 | 38.62 | 24.46 | 16.34 | 50.99 | 92.8 |
| PMTAG | | | | | | | |
| Polyol | | | | | | | |
| LF(D2)- | −40.90 | — | 38.41 | 18.86 | 10.55 | 42.17 | 93.3 |
| PMTAG | | | | | | | |
| Polyol | | | | | | | |

^{a}Shoulder peak

### Crystallization and Melting Behavior of SF-PMTAG Polyols

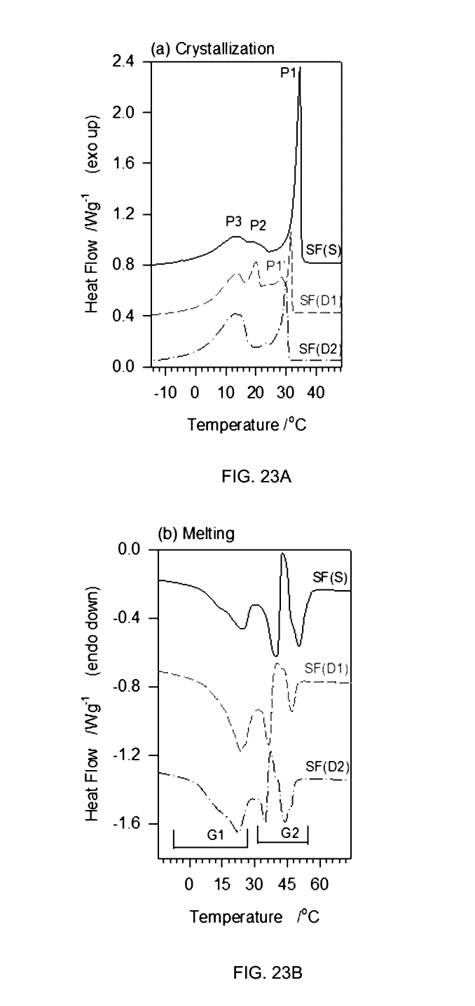

The crystallization and heating profiles (both at 5° C./min) of SF-PMTAG Polyols are shown in FIGS. 23A and 23B, respectively. The corresponding thermal data are listed in Table 22.

Unlike the polyols from the liquid fractions, the cooling thermograms of all the polyols from the solid fractions presented three peaks (FIG. 23A), indicating the presence of both the high and low melting fractions of the polyols. The onset temperature of crystallization (D2:˜31° C., D1:˜32° C. and S:˜35° C.) and offset temperature of melting (˜49, 50 and 57° C.) indicate that SF-PMTAG Polyols are not liquid at ambient and sub ambient temperature. The heating thermogram of the SF-PMTAG Polyols displayed two corresponding groups of endothermic events (G1 and G2 in FIG. 23b FIG. 23B), separated by a large recrystallization event indicating that polymorphic transformation mediated by melt occur with the SF-PMTAG Polyols.

TABLE 22
Thermal data of SF-PMTAG Polyols obtained on cooling
and heating (both at 5° C./min). Onset (Tₒₙ), offset (T_{off}),
and peak temperatures (T₁₋₃), Enthalpy of crystallization (Δ
H_{C}), and Enthalpy of melting (ΔH_{M}).

| | | | | | | | Enthalpy |
|---|---|---|---|---|---|---|---|
| | Temperature (° C.) | | | | | | (J/g) |
| Cooling | | | | | | | |
| | Tₒₙ | T₁ | T₁′ | T₂ | T₃ | T_{off} | ΔH_{C} |
| SF(D1)- | 32.09 | 31.61 | 28.27 | 20.05 | 13.40 | −1.17 | 113 |
| PMTAG | | | | | | | |
| Polyol | | | | | | | |
| SF(S)- | 35.07 | 34.63 | — | 19.48 | 13.06 | −3.46 | 107 |
| PMTAG | | | | | | | |
| Polyol | | | | | | | |
| SF(D2)- | 30.84 | 29.94 | — | 23.30 | 14.00 | −4.11 | 100 |
| PMTAG | | | | | | | |
| Polyol | | | | | | | |
| Heating | | | | | | | |
| | T_{off} | T₁ | T₂ | T₃ | T₄ | Tₒₙ | ΔH_{M} |
| SF(D1)- | 49.78 | 47.18 | 36.65 | 25.60^{a} | 23.83 | 2.79 | 101 |
| PMTAG | | | | | | | |
| Polyol | | | | | | | |
| SF(S)- | 56.48 | 50.36 | 39.83 | 24.32 | 14.61^{a} | 3.41 | 102 |
| PMTAG | | | | | | | |
| Polyol | | | | | | | |
| SF(D2)- | 48.50 | 44.12 | 34.61 | 22.66 | 12.53 | 4.03 | 112 |
| PMTAG | | | | | | | |
| Polyol | | | | | | | |

^{a}Shoulder peak

### Comparison of the Crystallization and Melting of SF and LF-PMTAG Polvols

LF- and SF-PMTAG Polyols presented significant differences in their cooling and heating thermograms, particularly prominently for those synthesized from the fractions of method D2 where the thermal events associated with the highest melting components were absent. The polyols made from the solid fractions crystallized at higher temperatures than their liquid fraction counterpart with differences of 3, 5, and 14° C. for D1, S and D2 polyols, respectively. The differences in crystallization behavior between the polyols made from the solid and liquid fractions manifested in the melting thermograms by extra high temperature endotherms, higher offsets of melting and significant polymorphic activity (recrystallization peak in the SF-PMTAG polyols (exotherms in FIG. 23B). These differences are a consequence of the differences in composition of their starting materials.

### Solid Fat Content of LF- and SF-PMTAG Polyols

### Solid Fat Content of LF-PMTAG Polyols

Solid Fat Content (SFC) versus temperature curves on cooling (5° C./min) and heating (5° C./min) of the polyols from the liquid fractions of PMTAG obtained by dry, solvent and melt fractionation are shown in FIGS. 24A and 24B, respectively. Extrapolated induction and offset temperatures as determined by SFC during cooling and heating are listed in Table 23. As can be seen in FIG. 24A, the SFC cooling curves of LF(S)-PMTAG Polyol presented two segments indicative of a two-step solidification process, whereas, LF(D1)- and LF(D2)-PMTAG Polyols presented only one segment. The SFC heating curves of the polyols mirrored the SFC cooling curves, with also two identifiable segments (segments 1 and 2 in FIG. 24B) for LF(S)-PMTAG Polyol and a single segment for LF(D1)- and LF(D2)-PMTAG Polyol. These SFC data indicate the presence of high and low temperature polyol fraction in LF(S)-PMTAG Polyol but not LF(D1)- and LF(D2)-PMTAG Polyols. The induction temperature of LF(S)-PMTAG Polyol (36.1° C.) was somewhat higher than LF(D1)-PMTAG Polyol (33.5° C.) and LF(D2)-PMTAG Polyol (25.8° C.).

TABLE 23
Extrapolated induction and offset temperatures (T_{ind}, Tₛ, respectively)
of LF(D1)- and LF(S)-PMTAG Polyols as determined by SFC

| | |
|---|---|
| Cooling | Heating |

| Temperature (° C.) | T_{ind} | Tₛ | T_{ind} | Tₛ |
|---|---|---|---|---|
| LF(D1)-PMTAG Polyol | 33.5 | −0.8 | −8.5 | 39.9 |
| LF(S)-PMTAG Polyol | 36.1 | −3 | −2.8 | 50.1 |
| LF(D2)-PMTAG Polyol | 25.8 | 0.5 | −0.1 | 40.2 |

### Solid Fat Content of SF-PMTAG Polvols

Solid Fat Content (SFC) versus temperature curves on cooling (5° C./min) and heating (5° C./min) of the polyols from the solid fractions of PMTAG are shown in FIGS. 25A and 25B, respectively. Extrapolated induction (T_{ind}^{c}) and completion of solidification (Tₛ), and onset and offset temperatures of melting (Tₒₙ^{M} and T_{off}^{M}) as determined by SFC are listed in Table 24.

As can be seen in FIG. 25A, the SFC cooling curves of the polyol presented two segments indicative of a two-step solidification process, corroborating the DSC. However, the segments were much less defined for SF(D1)-PMTAG Polyol than the two others. The SFC heating curves of the polyols mirrored the SFC cooling curves, with also two segments (segments 1 and 2 in FIG. 25B) that are also identifiable much more easily for SF(S)- and SF(D2)- than SF(D1)-PMTAG Polyols. SF(S)-PMTAG Polyol presented a T_{ind}^{c} (˜41° C.) somewhat higher than SF(S)- and SF(D2)-PMTAG Polyols (˜37° C.) but much lower offset of melting (˜45° C. compared to ˜55° C.).

TABLE 24
Extrapolated induction and offset temperatures of solidification
(T_{ind}^{c}, Tₛ, respectively) and melting (T_{ind}^{M} and T_{off}^{M}, respectively)
of SF(D1)- and SF(S)-PMTAG Polyols as determined by SFC

| | |
|---|---|
| Cooling | Heating |

| Temperature (° C.) | T_{ind}^{c} | Tₛ | Tₒₙ^{M} | T_{off}^{M} |
|---|---|---|---|---|
| SF(D1)-PMTAG Polyol | 36.8 | −7.5 | −8.1 | 45.2 |
| SF(S)-PMTAG Polyol | 40.9 | −4.3 | −7.8 | 55.1 |
| SF(D2)-PMTAG Polyol | 36.6 | −10.7 | −12.9 | 45.1 |

### Flow Behavior and Viscosity of LF- and SF-PMTAG Polyols

### Flow Behavior and Viscosity of LF-PMTAG Polyols

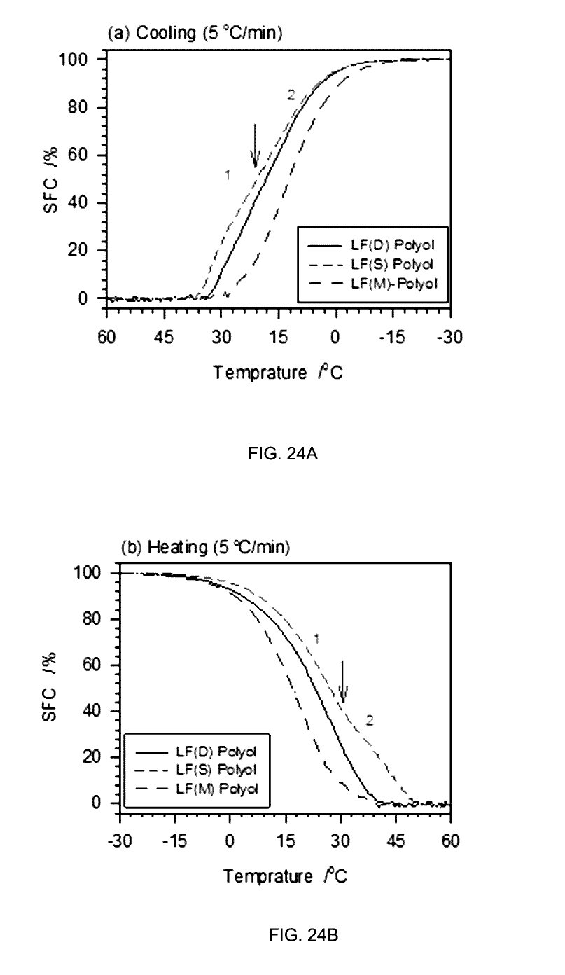

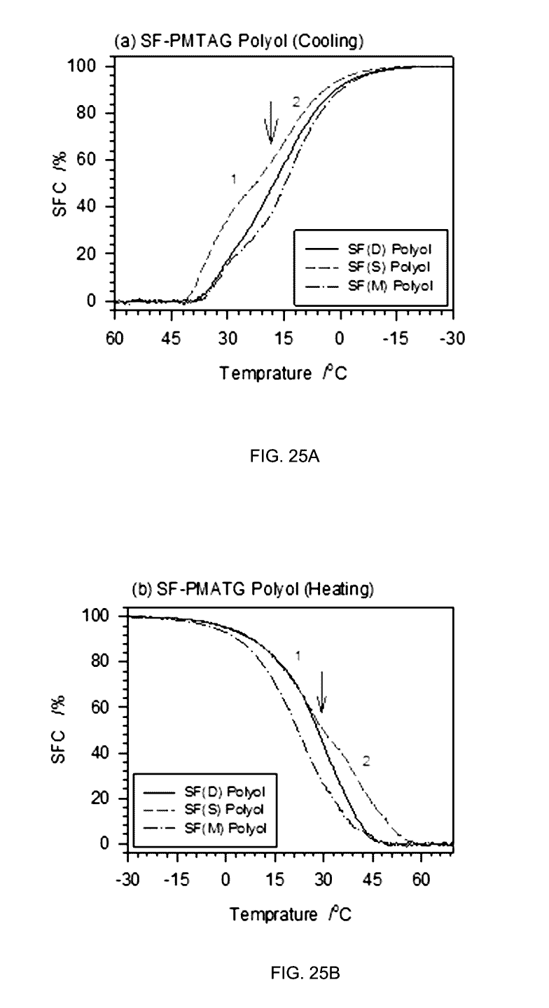

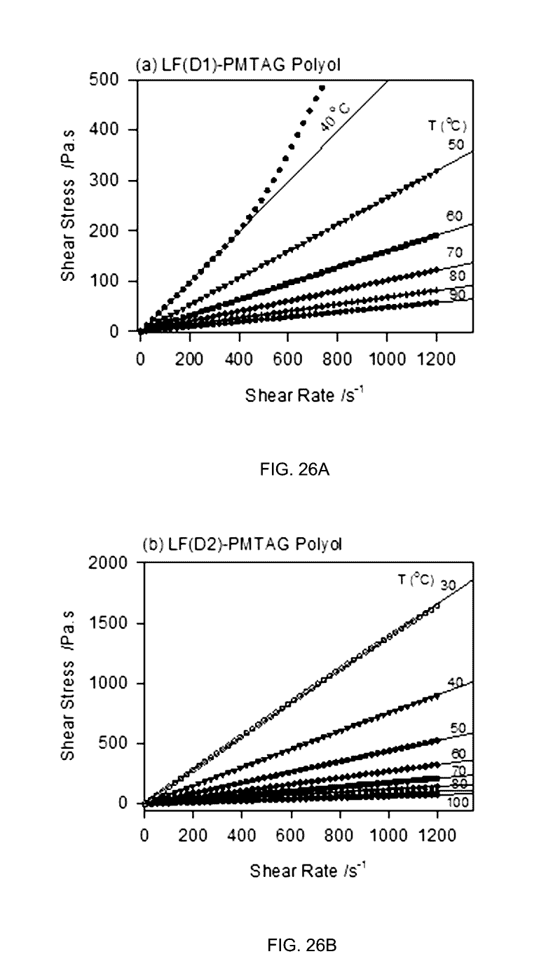

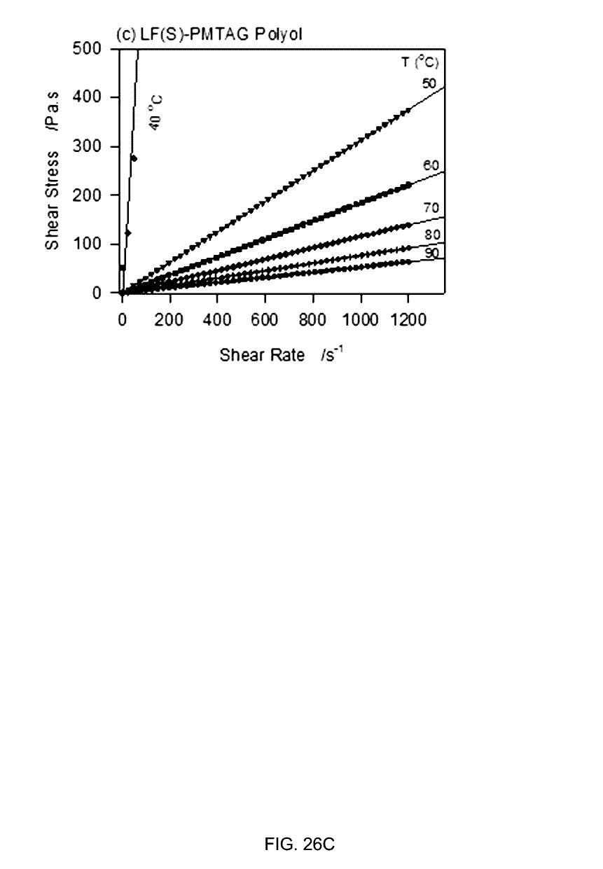

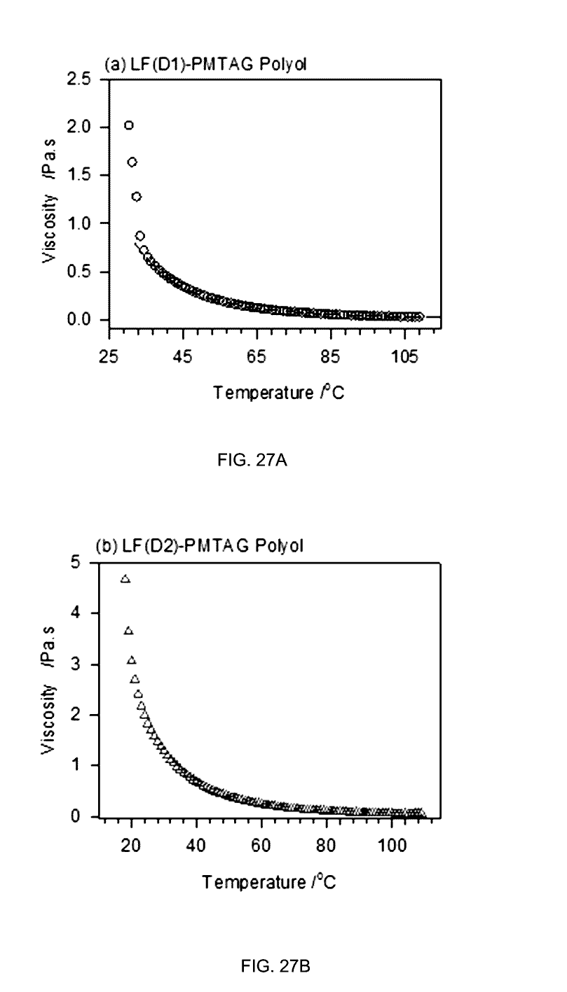

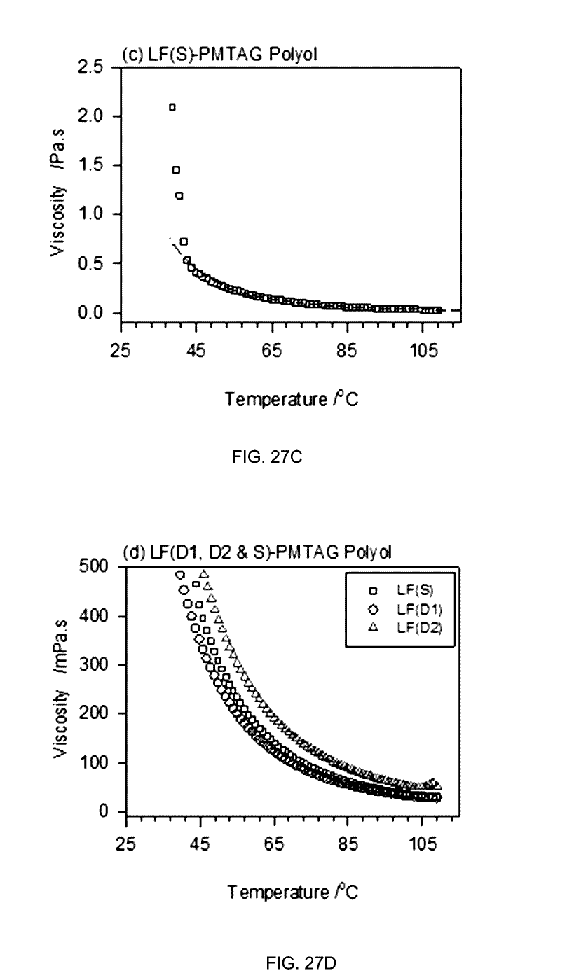

FIGS. 26A, 26B, and 26C show shear rate—shear stress curves obtained at different temperatures for LF(D1)- LF(S)- and LF(D2)-PMTAG Polyols, respectively. Fits to the Herschel-Bulkley (Eq. 1) model are included in the figures. FIGS. 27A, 27B, and 27C show the viscosity versus temperature curves obtained during cooling at 1° C./min for LF(D1)-, LF(S) and LF(D2)-PMTAG Polyols, respectively. Viscosity versus temperature graphs of LF(S)-, LF(D1)- and LF(D2)-PMTAG Polyols are shown together in FIG. 27D for comparison purposes.

The power index values (n) obtained for LF-PMTAG Polyol at temperatures above the onset temperature of crystallization (Tₒₙ) were approximately equal to 1, indicating a Newtonian behavior in the whole range of the used shear rates. The data collected below Tₒₙ (not shown) indicated that the sample has crystallized.

The viscosity versus temperature of liquid PMTAG Polyol obtained using the ramp procedure presented the typical exponential behavior of liquid hydrocarbons. As can be seen in FIG. 27D, LF(S)-PMTAG Polyol displayed higher viscosity at all temperatures. The difference which is as high as ˜300 mPa·s at 42° C., decreased exponentially with increasing temperature to reach 70 mPa·s at 45° C. and 3.5 mPa·s at 100° C.

### Flow Behavior and Viscosity of SF-PMTAG Polyols

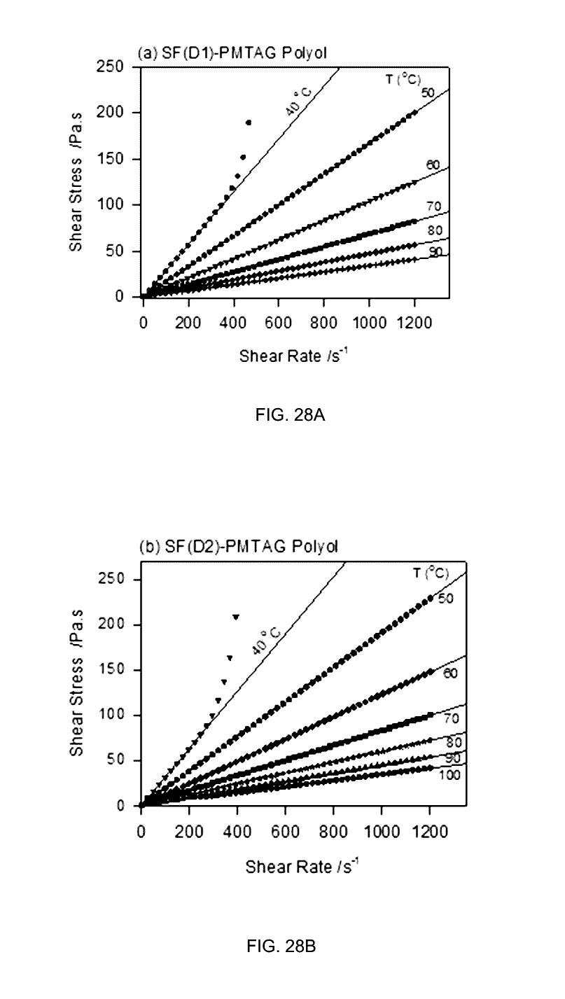

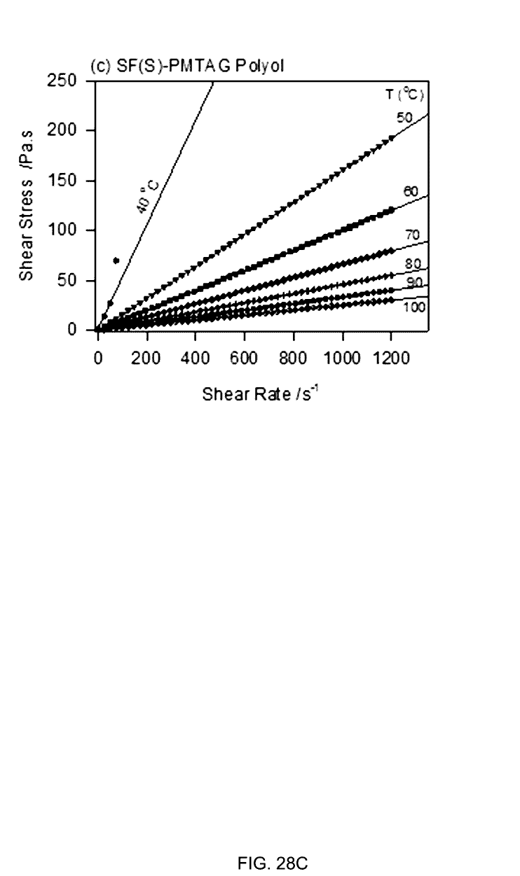

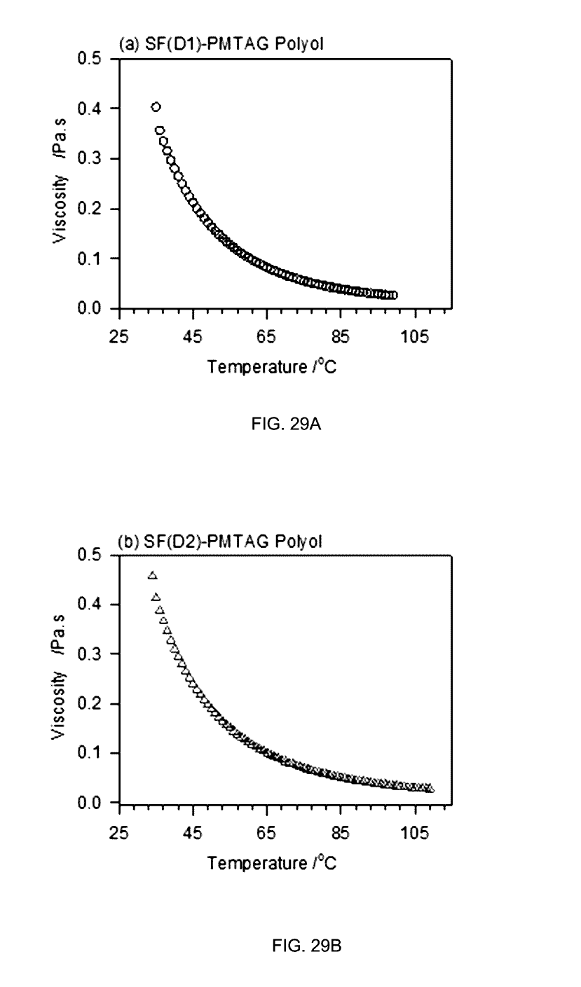

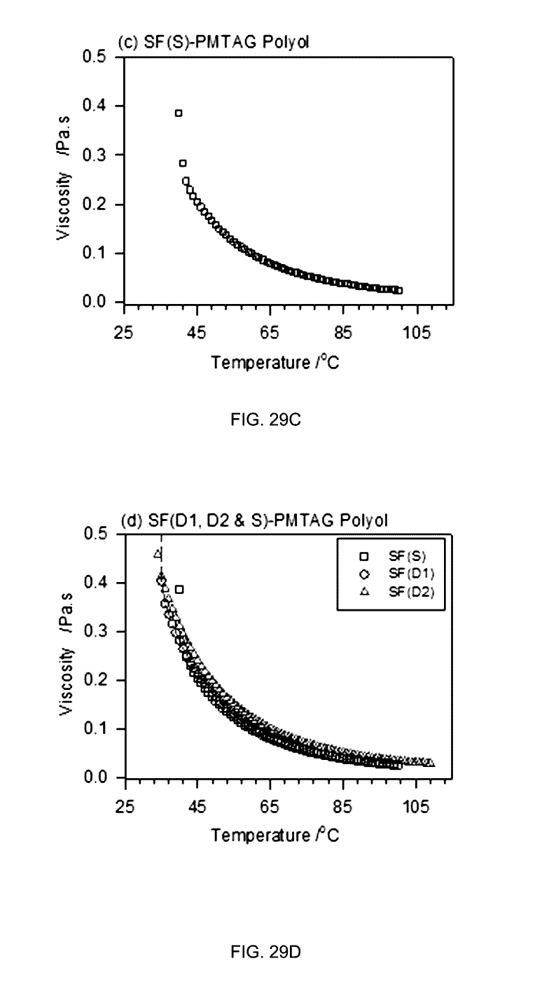

FIGS. 28A, 28B, and 28C show shear rate—shear stress curves obtained at different temperatures for SF(D1)- SF(S)- and SF(D2)-PMTAG Polyols, respectively. Fits to the Herschel-Bulkley (Eq. 1) model are included in the figures. FIGS. 29A, 29B, and 29C show the viscosity versus temperature curves obtained during cooling at 1° C./min for SF(D1)-, SF(S)- and SF(D2)-PMTAG Polyols, respectively. The three curves are shown together in FIG. 29D for comparison purposes.

As indicated by the values obtained for the power index (n), the PMTAG Polyols presented a Newtonian behavior in the whole range of the used shear rates above the onset temperature of crystallization (Tₒₙ). The data collected at the closest temperature to Tₒₙ (40° C.) indicate a Newtonian behavior only for small shear rates (lower than ˜100 s⁻¹ for SF(S)-PMTAG Polyol and ˜300 s−1 for the two others). The data collected below 40° C. (not shown) indicated that the sample has crystallized.

The viscosity versus temperature of liquid MTAG Polyol obtained using the ramp procedure presented the typical exponential behavior of liquid hydrocarbons. As can be seen, the Polyols made from the SF-PMTAG displayed almost the same viscosity at temperatures above the onset of crystallization. The difference in viscosity between SF(S) and SF(D1)-PMTAG Polyols was only ˜8 mPa·s at 40° C. and ˜0.7 mPa·s at 100° C.

### Comparison of Viscosity of LF(D)- and LF(S)-PMTAG Polyols

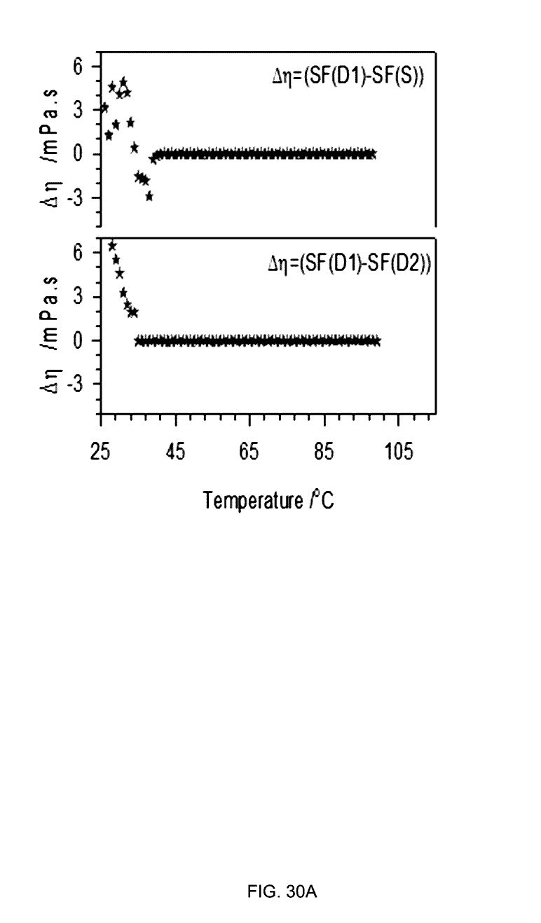

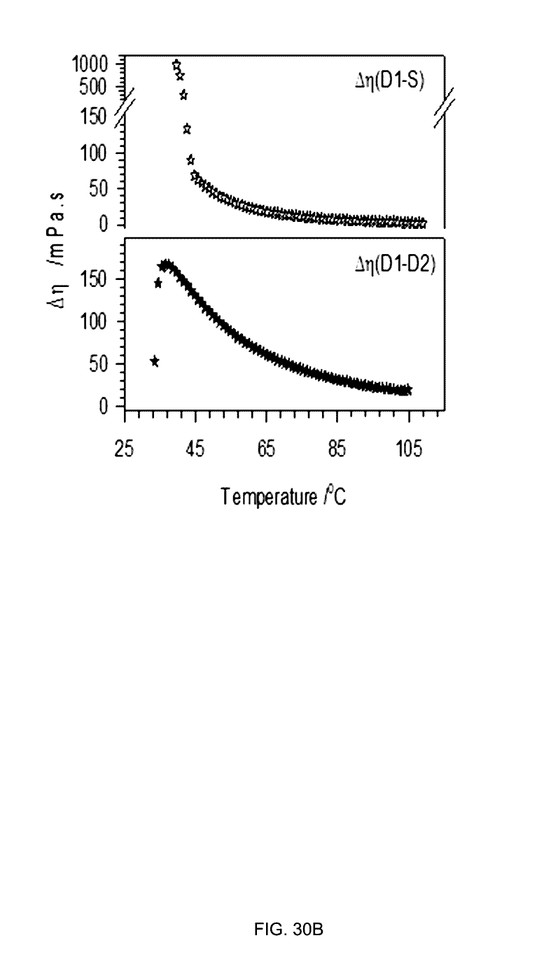

Viscosity difference versus temperature graphs between the solid fractions and between the liquid fractions are shown in FIGS. 30A and 30B. As can be seen in FIG. 30A, there was practically no significant difference in viscosity between the solid fractions below the onset temperature of crystallization. LF(D2)-PMTAG Polyol presented the highest viscosity at all temperatures below the onset temperature of crystallization, followed by LF(S)-PMTAG Polyol and LF(D1)-PMTAG Polyol. The difference between the liquid fractions decreased exponentially with increasing temperature (FIG. 30B). It was as high as ˜300 mPa·s at 42° C., reached 70 mPa·s at 45° C. and 3.5 mPa·s at 100° C. in the case of LF(S)-PMTAG Polyol and LF(D1)-PMTAG Polyol (Upper panel in FIG. 30B).

Polyurethane Foams from Polyols of PMTAG Fractions

### Polyurethane Foam Polymerization

Polyurethanes are one of the most versatile polymeric materials with regards to both processing methods and mechanical properties. The proper selection of reactants enables a wide range of polyurethanes (PU) elastomers, sheets, foams etc. Polyurethane foams are cross linked structures usually prepared based on a polymerization addition reaction between organic isocyanates and polyols, as generally shown in Scheme 8 below. Such a reaction may also be commonly referred to as a gelation reaction.

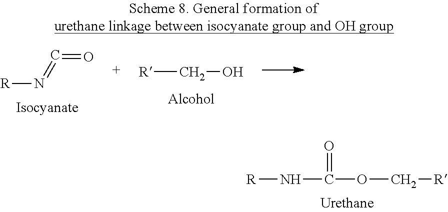

A polyurethane is a polymer composed of a chain of organic units joined by the carbamate or urethane link. Polyurethane polymers are usually formed by reacting one or more monomers having at least two isocyanate functional groups with at least one other monomer having at least two isocyanate-reactive groups, i.e. functional groups which are reactive towards the isocyanate function. The isocyanate (“NCO”) functional group is highly reactive and is able to react with many other chemical functional groups. In order for a functional group to be reactive to an isocyanate functional group, the group typically has at least one hydrogen atom which is reactive to an isocyanate functional group. A polymerization reaction is presented in Scheme 9, using a hexol structure as an example.

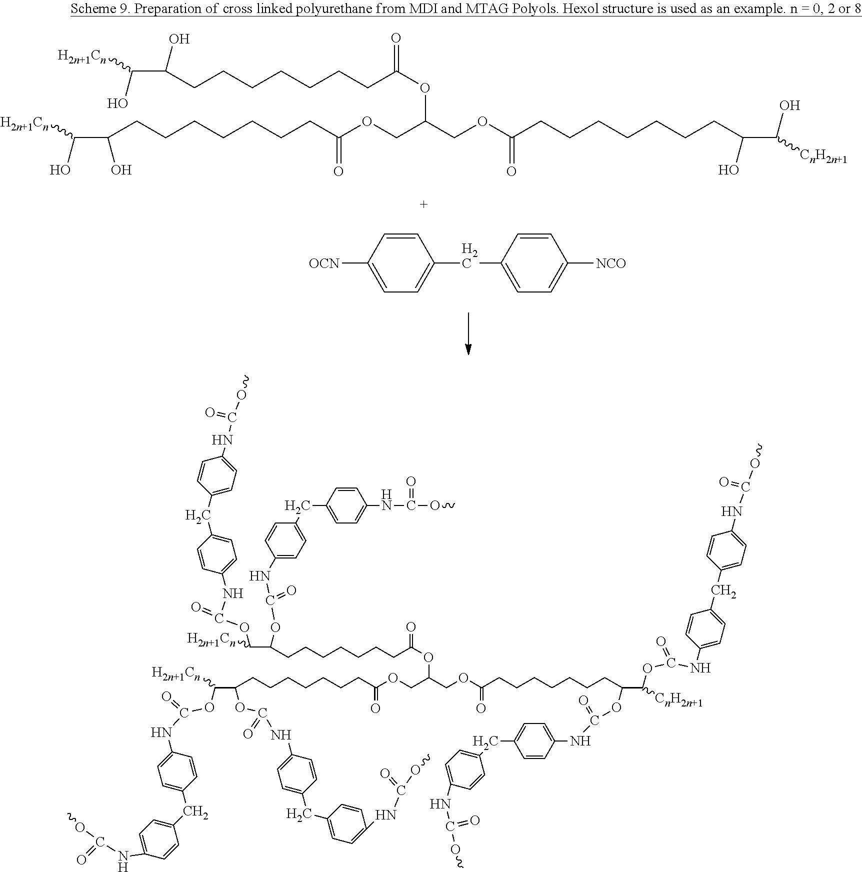

In addition to organic isocyanates and polyols, foam formulations often include one or more of the following non-limiting components: cross-linking components, blowing agents, cell stabilizer components, and catalysts. In some embodiments, the polyurethane foam may be a flexible foam or a rigid foam.

### Organic Isocyanates

The polyurethane foams of the present invention are derived from an organic isocyanate compound. In order to form large linear polyurethane chains, di-functional or polyfunctional isocyanates are utilized. Suitable polyisocyanates are commercially available from companies such as, but not limited to, Sigma Aldrich Chemical Company, Bayer Materials Science, BASF Corporation, The Dow Chemical Company, and Huntsman Chemical Company. The polyisocyanates of the present invention generally have a formula R(NCO)ₙ, where n is between 1 to 10, and wherein R is between 2 and 40 carbon atoms, and wherein R contains at least one aliphatic, cyclic, alicyclic, aromatic, branched, aliphatic- and alicyclic-substituted aromatic, aromatic-substituted aliphatic and alicyclic group. Examples of polyisocyanates include, but are not limited to diphenylmethane-4,4′-diisocyanate (MDI), which may either be crude or distilled; toluene-2,4-diisocyanate (TDI); toluene-2,6-diisocyanate (TDI); methylene bis (4-cyclohexylisocyanate (H₁₂MDI); 3-isocyanatomethyl-3,5,5-trimethyl-cyclohexyl isocyanate (IPDI); 1,6-hexane diisocyanate (HDI); naphthalene-1,5-diisocyanate (NDI); 1,3- and 1,4-phenylenediisocyanate; triphenylmethane-4,4′,4″-triisocyanate; polyphenylpolymethylenepolyisocyanate (PMDI); m-xylene diisocyanate (XDI); 1,4-cyclohexyl diisocyanate (CHDI); isophorone diisocyanate; isomers and mixtures or combinations thereof.

### Polyols

The polyols used in the foams described herein are based on the fractions of metathesized triacylglycerol (MTAG) derived from natural oils, including palm oil. The synthesis of the MTAG Polyol was described earlier, and involves epoxidation and subsequent hydroxylation of a fraction of an MTAG derived from a natural oil, including palm oil.

### Cross-Linking Components and Chain Extenders

Cross-linking components or chain extenders may be used if needed in preparation of polyurethane foams. Suitable cross-linking components include, but are not limited to, low-molecular weight compounds containing at least two moieties selected from hydroxyl groups, primary amino groups, secondary amino groups, and other active hydrogen-containing groups which are reactive with an isocyanate group. Crosslinking agents include, for example, polyhydric alcohols (especially trihydric alcohols, such as glycerol and trimethylolpropane), polyamines, and combinations thereof. Non-limiting examples of polyamine crosslinking agents include diethyltoluenediamine, chlorodiaminobenzene, diethanolamine, diisopropanolamine, triethanolamine, tripropanolamine, 1,6-hexanediamine, and combinations thereof. Typical diamine crosslinking agents comprise twelve carbon atoms or fewer, more commonly seven or fewer. Other cross-linking agents include various tetrols, such as erythritol and pentaerythritol, pentols, hexols, such as dipentaerythritol and sorbitol, as well as alkyl glucosides, carbohydrates, polyhydroxy fatty acid esters such as castor oil and polyoxy alkylated derivatives of poly-functional compounds having three or more reactive hydrogen atoms, such as, for example, the reaction product oftrimethylolpropane, glycerol, 1,2,6-hexanetriol, sorbitol and other polyols with ethylene oxide, propylene oxide, or other alkylene epoxides or mixtures thereof, e.g., mixtures of ethylene and propylene oxides.

Non-limiting examples of chain extenders include, but are not limited to, compounds having hydroxyl or amino functional group, such as glycols, amines, diols, and water. Specific non-limiting examples of chain extenders include ethylene glycol, diethylene glycol, propylene glycol, dipropylene glycol, 1,4-butanediol, 1,3-butanediol, 1,5-pentanediol, neopentyl glycol, 1,6-hexanediol, 1,10-decanediol, 1,12-dodecanediol, ethoxylated hydroquinone, 1,4-cyclohexanediol, N-methylethanolamine, N-methylisopropanolamine, 4-aminocyclohexanol, 1,2-diaminoethane, 2,4-toluenediamine, or any mixture thereof.

### Catalyst

The catalyst component can affect the reaction rate and can exert influence on the open celled structures and the physical properties of the foam. The proper selection of catalyst (or catalysts) appropriately balance the competing interests of the blowing and polymerization reactions. A correct balance is needed due to the possibility of foam collapse if the blow reaction proceeds relatively fast. On the other hand, if the gelation reaction overtakes the blow reaction, foams with closed cells might result and this might lead to foam shrinkage or ‘pruning’. Catalyzing a polyurethane foam, therefore, involves choosing a catalyst package in such a way that the gas produced becomes sufficiently entrapped in the polymer. The reacting polymer, in turn, must have sufficient strength throughout the foaming process to maintain its structural integrity without collapse, shrinkage, or splitting.

The catalyst component is selected from the group consisting of tertiary amines, organometallic derivatives or salts of, bismuth, tin, iron, antimony, cobalt, thorium, aluminum, zinc, nickel, cerium, molybdenum, vanadium, copper, manganese and zirconium, metal hydroxides and metal carboxylates. Tertiary amines may include, but are not limited to, triethylamine, triethylenediamine, N,N,N′,N′-tetramethylethylenediamine, N,N,N′,N′-tetraethylethylenediamine, N-methylmorpholine, N-ethylmorpholine, N,N,N′,N′-tetramethylguanidine, N,N,N′,N′-tetramethyl-1,3-butanediamine, N,N-dimethylethanolamine, N,N-diethylethanolamine. Suitable organometallic derivatives include di-n-butyl tin bis(mercaptoacetic acid isooctyl ester), dimethyl tin dilaurate, dibutyl tin dilaurate, dibutyl tin sulfide, stannous octoate, lead octoate, and ferric acetylacetonate. Metal hydroxides may include sodium hydroxide and metal carboxylates may include potassium acetate, sodium acetate or potassium 2-ethylhexanoate.

### Blowing Agents

Polyurethane foam production may be aided by the inclusion of a blowing agent to produce voids in the polyurethane matrix during polymerization. The blowing agent promotes the release of a blowing gas which forms cell voids in the polyurethane foam. The blowing agent may be a physical blowing agent or a chemical blowing agent. The physical blowing agent can be a gas or liquid, and does not chemically react with the polyisocyanate composition. The liquid physical blowing agent typically evaporates into a gas when heated, and typically returns to a liquid when cooled. The physical blowing agent typically reduces the thermal conductivity of the polyurethane foam. Suitable physical blowing agents for the purposes of the invention may include liquid carbon dioxide, acetone, and combinations thereof. The most typical physical blowing agents typically have a zero ozone depletion potential. Chemical blowing agents refers to blowing agents which chemically react with the polyisocyanate composition.

Suitable blowing agents may also include compounds with low boiling points which are vaporized during the exothermic polymerization reaction. Such blowing agents are generally inert or they have low reactivity and therefore it is likely that they will not decompose or react during the polymerization reaction. Examples of blowing agents include, but are not limited to, water, carbon dioxide, nitrogen gas, acetone, and low-boiling hydrocarbons such as cyclopentane, isopentane, n-pentane, and their mixtures. Previously, blowing agents such as chlorofluorocarbons (CFCs), hydrofluorocarbons (HFCs), hydrochlorofluorocarbons (HCFCs), fluoroolefins (FOs), chlorofluoroolefins (CFOs), hydrofluoroolefins (HFOs), and hydrochlorfluoroolefins (HCFOs), were used, though such agents are not as environmentally friendly. Other suitable blowing agents include water that reacts with isocyanate to produce a gas, carbamic acid, and amine, as shown below in Scheme 10.

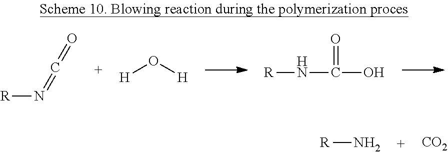

Various methods were adopted in the present study to produce rigid and flexible foams from fractions of PMTAG and Polyols derived therefrom.

### Cell Stabilizers

Cell stabilizers may include, for example, silicone surfactants or anionic surfactants. Examples of suitable silicone surfactants include, but are not limited to, polyalkylsiloxanes, polyoxyalkylene polyol-modified dimethylpolysiloxanes, alkylene glycol-modified dimethylpolysiloxanes, or any combination thereof. Suitable anionic surfactants include, but are not limited to, salts of fatty acids, salts of sulfuric acid esters, salts of phosphoric acid esters, salts of sulfonic acids, and combinations of any of these. Such surfactants provide a variety of functions, reducing surface tension, emulsifying incompatible ingredients, promoting bubble nucleation during mixing, stabilization of the cell walls during foam expansion, and reducing the defoaming effect of any solids added. Of these functions, a key function is the stabilization of the cell walls, without which the foam would behave like a viscous boiling liquid.

### Additional Additives

If desired, the polyurethane foams can have incorporated, at an appropriate stage of preparation, additives such as pigments, fillers, lubricants, antioxidants, fire retardants, mold release agents, synthetic rubbers and the like which are commonly used in conjunction with polyurethane foams.

### Flexible and Rigid Foam Embodiments

In some embodiments, the polyurethane foam may be a flexible foam, where such composition comprises (i) at least one polyol composition derived from a fraction of a natural oil based metathesized triacylglycerols component; (ii) at least one polyisocyanate component, wherein the ratio of hydroxy groups in said at least one polyol to isocyanate groups in said at least one polyisocyanate component is less than 1; (iii) at least one blowing agent; (iv) at least one cell stabilizer component; and (v) at least one catalyst component; wherein the composition has a wide density range, which can be between about 85 kgm⁻³ and 260 kgm⁻³, but can in some instances be much wider.

In other embodiments, the polyurethane foam may be a rigid foam, where the composition comprises (i) at least one polyol derived from a fraction of a natural oil based metathesized triacylglycerols component; (ii) at least one polyisocyanate component, wherein the ratio of hydroxy groups in said at least one polyol to isocyanate groups in said at least one polyisocyanate component is less than 1; (iii) at least one cross-linking component (iv) at least one blowing agent; (v) at least one cell stabilizer component; and (vi) at least one catalyst component; wherein the composition has a wide density range, which can be between about 85 kgm⁻³ and 260 kgm⁻³, but can in some instances be much wider.

### Analytical Methods for PMTAG Polyol Foam Analysis

The PMTAG Polyol foam was analyzed using different techniques. These techniques can be broken down into: (i) chemistry characterization techniques, including NCO value and Fourier Transform infrared spectroscopy (FTIR); and (ii) physical characterization methods, including thermogravimetric analysis (TGA), differential scanning calorimetry (DSC), scanning electron microscopy (SEM) and compressive test.

### Chemistry Characterization Techniques of PMTAG Polyol Foam

The amount of reactive NCO (% NCO) for diisocyanates was determined by titration with dibutylamine (DBA). MDI (2±0.3 g) was weighed into 250 ml conical flasks. 2N DBA solution (20 ml) was pipetted to dissolve MDI. The mixture is allowed to boil at 150° C. until the vapor condensate is at an inch above the fluid line. The flasks were cooled to RT and rinsed with methanol to collect all the products. 1 ml of 0.04% bromophenol blue indicator is then added and titrated against 1N HCl until the color changes from blue to yellow. A blank titration using DBA is also done.

The equivalent weight (EW) of the MDI is given by Eq. 2

$$EW = \frac{W \times 1000}{( {V}_{1} - {V}_{2} ) \times N} \qquad Eq . 2$$

where W is the weight of MDI in g, V₁ and V₂ are the volume of HCl for the blank and MDI samples, respectively. N is the concentration of HCl. The NCO content (%) is given by Eq. 3:

$$\% NCO content = \frac{42}{EW} \times 100 \qquad Eq . 3$$

FTIR spectra were obtained using a Thermo Scientific Nicolet 380 FT-IR spectrometer (Thermo Electron Scientific Instruments, LLC, USA) equipped with a PIKE MIRacle™ attenuated total reflectance (ATR) system (PIKE Technologies, Madison, Wis., USA.). Foam samples were loaded onto the ATR crystal area and held in place by a pressure arm, and sample spectra were acquired over a scanning range of 400-4000 cm⁻¹ for 32 repeated scans at a spectral resolution of 4 cm⁻¹.

### Physical Characterization Techniques of PMTAG Polyol Foam

TGA was carried out on a TGA Q500 (TA Instruments, DE, USA) equipped with a TGA heat exchanger (P/N 953160.901). Approximately 8.0-15.0 mg of sample was loaded in the open TGA platinum pan. The sample was heated from 25 to 600° C. under dry nitrogen at a constant rate of 10° C./min.

DSC measurements were run on a Q200 model (TA Instruments, New Castle, Del.) under a nitrogen flow of 50 mL/min. PMTAG Polyol Foam samples between 3.0 and 6.0 (±0.1) mg were run in hermetically sealed aluminum DSC pans. In order to obtain a better resolution of the glass transition, PMTAG Polyol foams were investigated using modulated DSC following ASTM E1356-03 standard. The sample was first equilibrated at −90° C. and heated to 150° C. at a constant rate of 5.0° C./min (first heating cycle), held at 150° C. for 1 min and then cooled down to −90° C. with a cooling rate of 5° C./min, and subsequently reheated to 150° C. at the same rate (second heating cycle). Modulation amplitude and period were 1° C. and 60 s, respectively. The “TA Universal Analysis” software was used to analyze the DSC thermograms.

A scanning electron microscope (SEM), model Tescan Vega II, was used under standard operating conditions (10 keV beam) to examine the pore structure of the foams. A sub-sample of approximately 2 cm×2 cm and 0.5 cm thick was cut from the center of each sample. The sample was coated with a thin layer of carbon (˜30 nm thick) using an Emitech K950X turbo evaporator to ensure electrical conductivity in the SEM chamber and prevent the buildup of electrons on the surface of the sample. All images were acquired using a secondary electron detector to show the surface features of the samples.

The compressive strength of the foams was measured at room temperature using a texture analyzer (Texture Technologies Corp, NJ, USA). Samples were prepared in cylindrical Teflon molds of 60-mm diameter and 36-mm long. The cross head speed was 3.54 mm/min with a load cell of 500 kgf or 750 kgf. The load for the rigid foams was applied until the foam was compressed to approximately 80% of its original thickness, and compressive strengths were calculated based on the 5, 6, 10 and 15% deformations. The load for the flexible foams was applied until the foam was compressed to approximately 35% of its original thickness, and compressive strengths were calculated based on 10, 25 and 50% deformation.

### Polymerization Conditions and Foams Produced

### General Materials

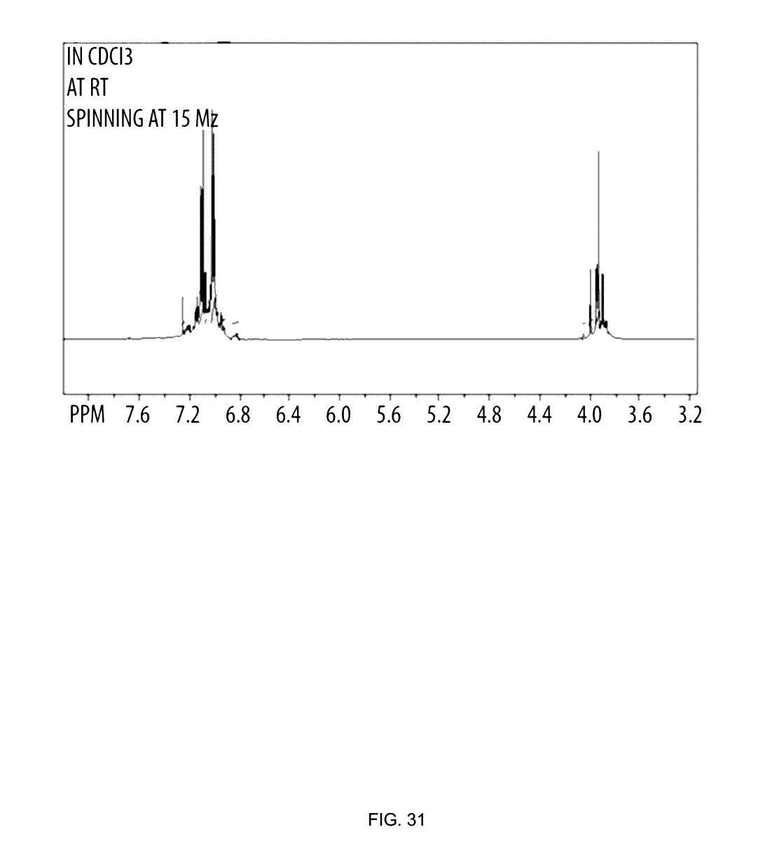

The materials used to produce the foams are listed in Table 25. The polyols were obtained from the liquid fractions of MTAG of palm oil as generally described above. A commercial isocyanate, methylene diphenyl diisocyanate (MDI) and a general-purpose silicone surfactant, polyether-modified (TEGOSTAB B-8404, Goldschmidt Chemical Canada) were used in the preparation. FIG. 31 shows the ¹H-NMR spectrum of MDI, and Table 26 presents the corresponding chemical shift values. The physical properties of the crude MDI are reported in Table 27.

TABLE 25
Materials used in the polymerization reaction

| Material | |
|---|---|
| Polyol | LF(D1)-PMTAG Polyol |
| Isocyanate | Crude MDI^{a} |
| Catalyst | DBTDL^{b}, 95% |
| | DMEA^{c}, 99.5% |
| Cross linker | Glycerin, 99.5% |
| Surfactant | TEGOSTAB ® B-8404^{d} |
| Blowing agent | CO₂ from addition of 2% deionized H₂O |

^{a}MDI: Diphenylmethane diisocynate, from Bayer Materials Science, Pittsburgh, PA
^{b}DBTDL: Dibutin Dilaurate, main catalyst, from Sigma Aldrich, USA
^{c}DMEA: N,N-Dimethylethanolamine, co-catalyst, from Fischer Chemical, USA
^{d}TEGOSTAB ® B-8404, Polyether-modified, a general-purpose silicone surfactant, from Goldschmidt Chemical, Canada

TABLE 26
¹H-NMR data of the diisocyanates; Chemical shift δ (ppm)
MDI

| | NCO at position of Benzene | | | CH₂ in | | | | |
|---|---|---|---|---|---|---|---|---|
| | 2 | 4 | 4 | isomer | | | | |
| Protons | p, o, m (CH═CH) | m(CH═CH) | o(CH═CH) | 2,2′ | 2,4′ | 4,4′ | Others | Oligomers |
| δ (ppm) | 7.1386-7.1599 | 7.0779-7.1275 | 7.0175-7.0384 | 4.04 | 3.9904 | 3.9420 | 3.8929 | 3.9253 |

TABLE 27
Physical properties of crude MDI as provided by the supplier.

| Property | Value |
|---|---|
| Form | Dark brown liquid |
| Boiling Point (° C.) | 208 |
| NCO content (% wt.) | 31.5 |
| | 31.4^{a} |
| Equivalent weight | 133 |
| | 133.8^{a} |
| Functionality | 2.4 |
| Viscosity @ 25° C. (mPas) | 200 |
| Bulk density (kgm⁻³) | 1234 |
| Composition | Polymeric MDI: 40-50% |
| | (4,4′ diphenylmethane diisocyanate): 30-40% |
| | MDI mixed isomers: 15-25% |

^{a}as measured

The hydroxyl value (OH value) and acid value of the polyols, measured using ASTM D1957-86 and ASTM D4662-03, respectively, are listed in Table 28. There were no free fatty acids detected by ¹H-NMR. There was also no signal that can be associated with the loss of free fatty acids in the TGA of the LF-PMTAG Polyols. The acid value reported in Table 28 was probably due to the hydrolysis of LF-PMTAG Polyol during the actual titration, which uses strong base as the titrant, with the result that the actual titration causes hydrolysis.

TABLE 28
OH and acid values of LF-PMTAG Polyol
used in the foams formulation

| | OH Value | Acid Value |
|---|---|---|
| Polyol | (mg KOH/g) | (mg KOH/g) |
| LF(D1)-PMTAG Polyol | 184 | <4 |
| LF(D2)-PMTAG Polyol | 170 | <2.3 |
| LF(S)-PMTAG Polyol | 182 | <4 |

Synthesis of Foams from LF-PMTAG Polyols

Rigid and flexible polyurethane foams of different densities were obtained using appropriate recipe formulations. The amount of each component of the polymerization mixture was based on 100 parts by weight of total polyol. The amount of MDI was taken based on the isocyanate index 1.2. All the ingredients, except MDI, were weighed into a beaker and MDI was added to the beaker using a syringe, and then mechanically mixed vigorously for ˜20 s. At the end of the mixing period, mixed materials was added into a cylindrical Teflon mold (60 mm diameter and 35 mm long) which was previously greased with silicone release agent and sealed with a hand tightened clamp. The sample was cured for four (4) days at 40° C. and post cured for one (1) day at room temperature.

Rigid Foam formulation was determined based on a total hydroxyl value of 450 mg KOH/g according to teachings known in the field. Table 29 presents the formulation recipe used to prepare the rigid and flexible foams. Note that in the case of rigid foams, around 14.5 or 15.3 parts of glycerin were added into the reaction mixture in order to reach the targeted hydroxyl value of 450 mg KOH/g. Flexible Foam formulation was based on a total hydroxyl value of 184 mg KOH/g according to teachings known in the field. In the case flexible foams, no glycerin was added into the reaction mixture, and the catalyst amount was fixed to 0.1 parts for proper control of the polymerization reaction.

TABLE 29
Formulation Recipes for Rigid and Flexible Foams

| | Rigid Foams | Flexible Foams |
|---|---|---|
| Ingredient | Parts | Parts |

| LF(D1)-PMTAG Polyol | | | 100 | 100 |
|---|---|---|---|---|
| OH:NCO ratio | | | 1:1.2 | 1:1.2 |
| | Glycerin | D1 | 14.5 | 0 |
| | | D2 | 15.3 | 0 |
| | | S | 14.7 | 0 |

| | | |
|---|---|---|
| Water | 2 | 2 |
| Surfactant | 2 | 2 |
| Catalyst | 1 | 0.1 |
| Co-catalyst | 1 | 0.1 |
| Mixing Temperature (° C.) | 40 | 40 |
| Oven Temperature (° C.) | 40 | 40 |

### LF-PMTAG Polyol Foams Produced

Two different rigid foams (LF(D1)-RF160 and LF(D1)-RF163, with densities of 160 and 163 kgm⁻³, respectively) and two different flexible foams (LF(D1)-FF160 and LF(D1)-FF165, with densities of 160 and 165 kgm⁻³, respectively) were prepared from LF(D1)-PMTAG Polyol.

One rigid foams (LF(D2)-RF167, with density of 167 kgm⁻³) and one flexible foam (LF(D2)-FF155, with density of 155 kgm⁻³) were prepared from LF(D2)-PMTAG Polyol.

Two different rigid foams (LF(S)-RF153 and LF(S)-RF166, with densities of 153 and 166 kgm⁻³, respectively) and two different flexible foams (LF(S)-FF155 and LF(S)-FF165, with densities of 155 and 165 kgm⁻³, respectively) were prepared from LF(S)-PMTAG Polyol.

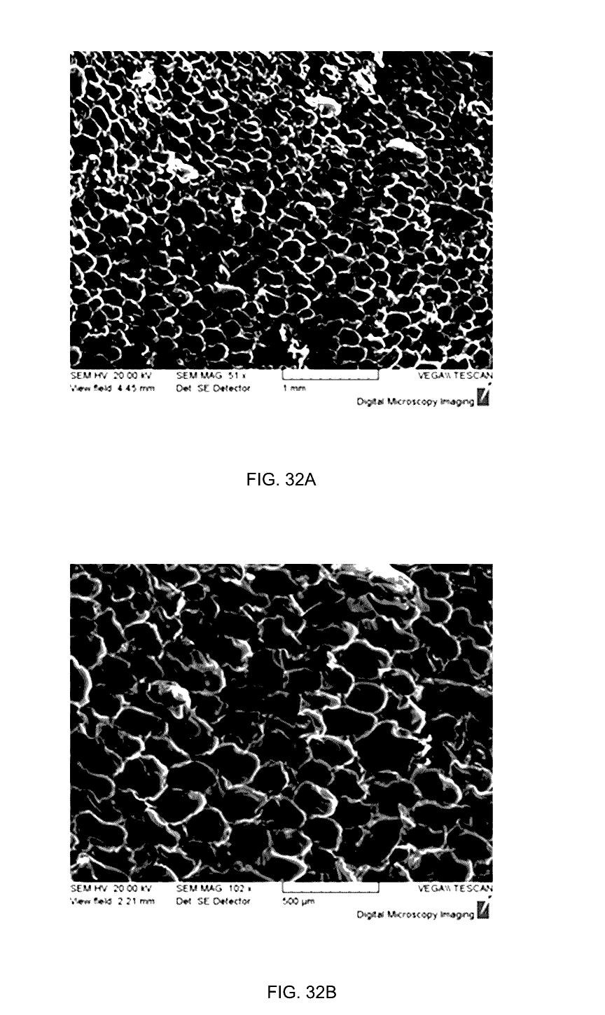

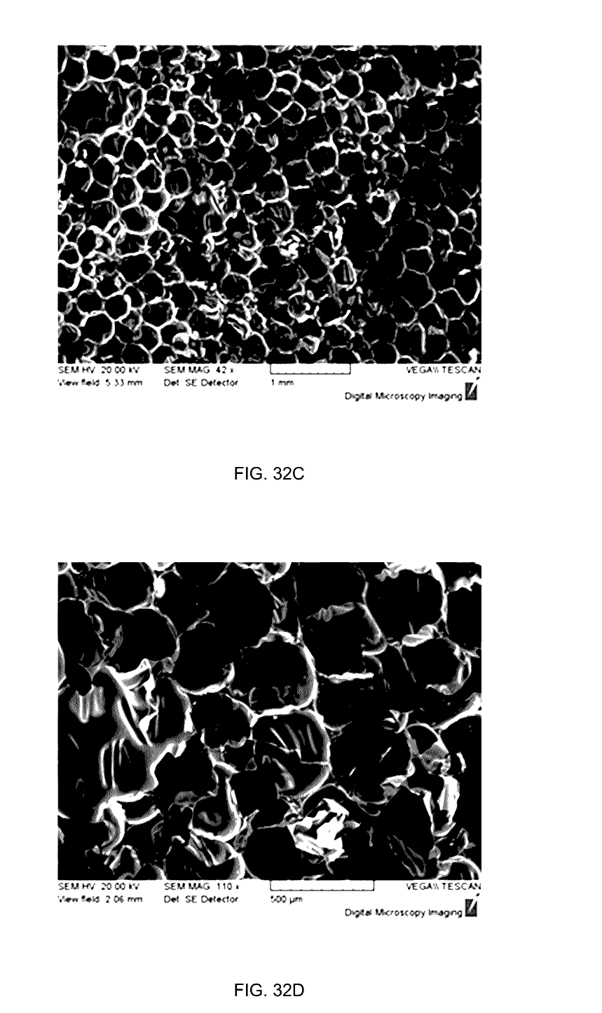

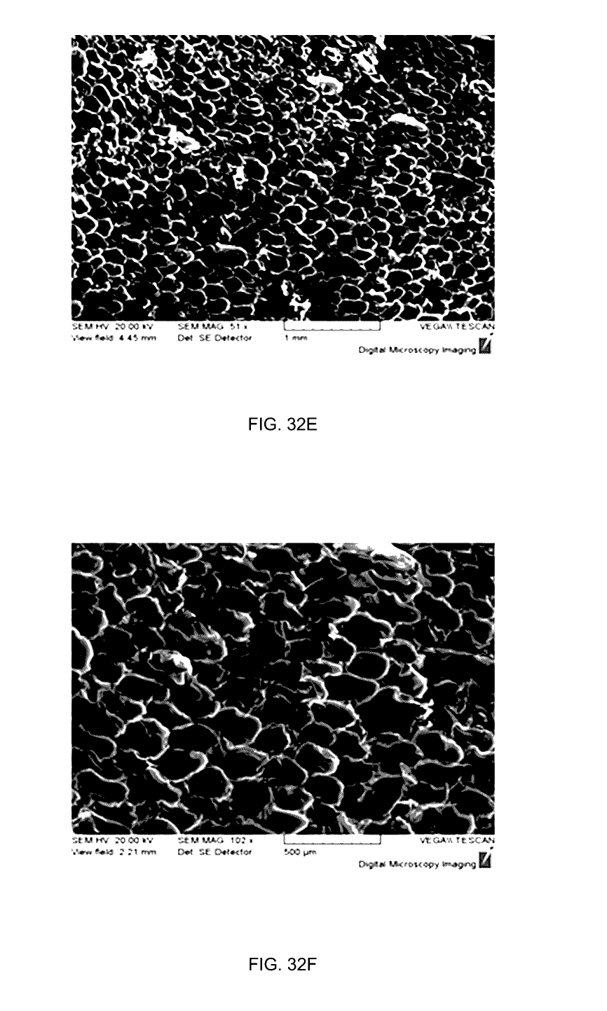

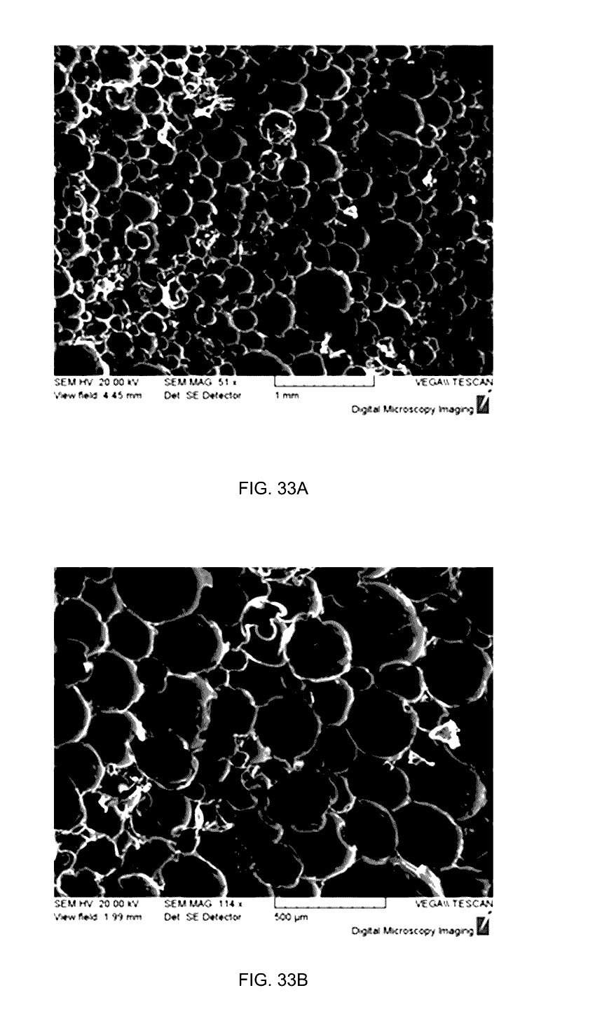

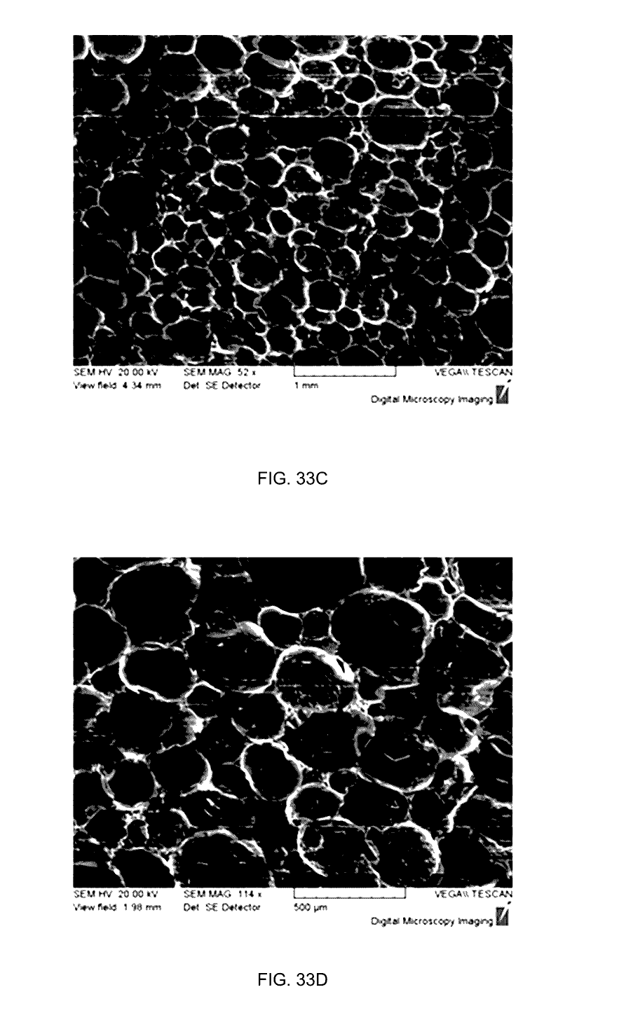

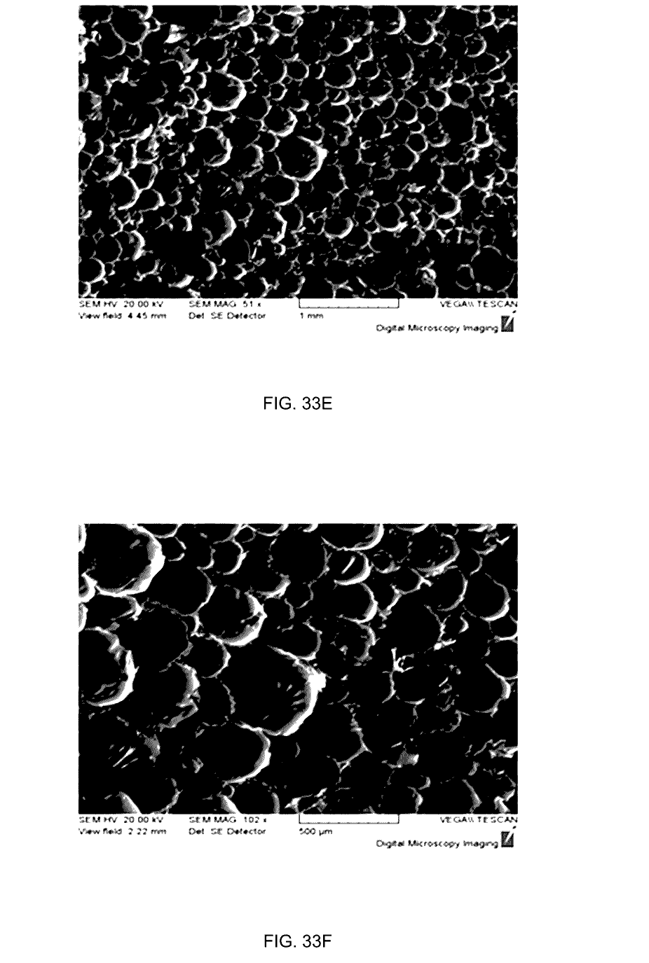

Pictures of the LF(D1)-, LF(D2)- and LF(S)-PMTAG Polyol foams (not shown) show the resulting foams appearing as very regular and smooth. The foams presented a homogenous closed cell structure elucidated through SEM micrographs, examples of which are shown in FIGS. 32A-32F for the rigid LF-PMTAG Polyol foams, respectively, and in FIGS. 33A-33F for the flexible LF-PMTAG Polyol foams, respectively.

### FTIR of LF-PMTAG Polyol Foams

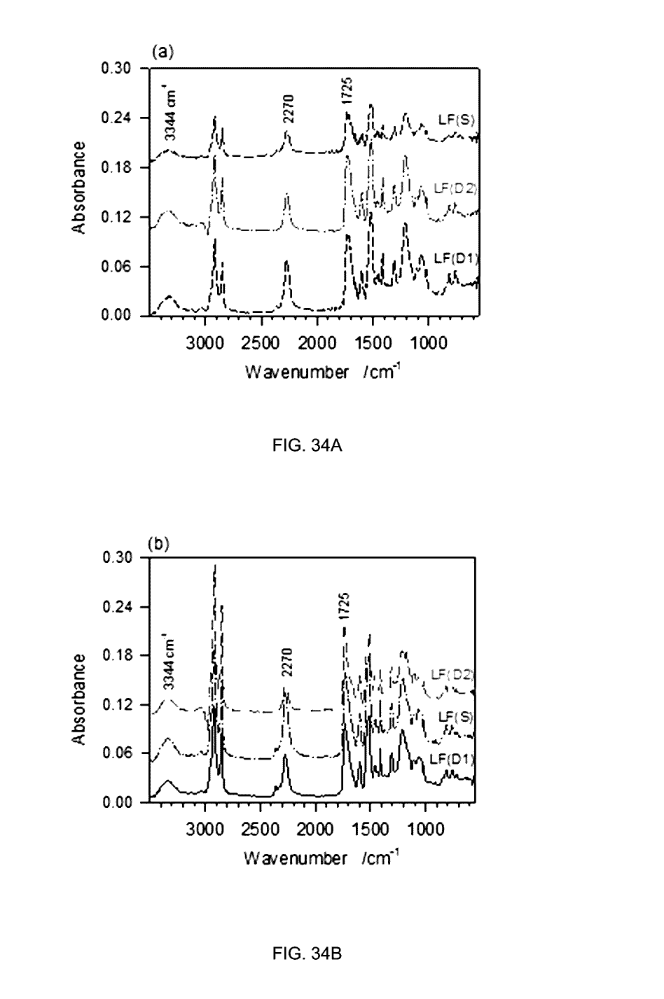

FTIR spectra typical of rigid and flexible LF-PMTAG Polyol Foams are shown in FIGS. 34A and 34B, respectively. Table 30 lists the characteristic vibrations of the foams. The broad absorption band observed at 3300-3400 cm⁻¹ in the foam is characteristic of NH group associated with the urethane linkage. The overlapping peaks between 1710 and 1735 cm⁻¹ suggest the formation of urea, isocyanurate and free urethane in the PMTAG Polyol foams.

The CH₂ stretching vibration is clearly visible at 2800-3000 cm⁻¹ region in the spectra. The band centered at 1700 cm⁻¹ is characteristic of C═O, which demonstrates the formation of urethane linkages. The band at 1744 cm⁻¹ is attributed to the C═O stretching of the ester groups. The sharp band at 1150-1160 cm⁻¹ and 1108-1110 cm⁻¹ are the O—C—C and C—C(═O)—O stretching bands, respectively, of the ester groups. The band at 1030-1050 cm⁻¹ is due to CH₂ bend.

TABLE 30
FTIR data of LF-PMTAG Polyol foams

| Moiety | Wavelengths (cm⁻¹) |
|---|---|
| H-bonded and free N—H groups | 3300-3400 |
| Free NCO | 2270 |
| Urea | 1717 |
| Isocyanurate | 1710 |
| Free Urethane | 1735 |

### Physical Properties of LF-PMTAG Polvol Foams

### Thermal Stability of LF-PMTAG Polyol Foams

The thermal stability of the LF-PMTAG Polyol foams was determined by TGA. Typical DTG curves of rigid and flexible LF-PMTAG Polyol Foams are shown in FIGS. 35A and 35B, respectively. The corresponding data (extrapolated onset and offset temperatures of degradation, temperature of degradation measured at 1, 5 and 10% decomposition, and the DTG peak temperatures) are provided in Table 31.

The initial step of decomposition indicated by the DTG peak at ˜300° C. with a total weight loss of 17% is due to the degradation of urethane linkages, which involves dissociations to the isocyanate and the alcohol, amines and olefins or to secondary amines. The second decomposition step in the range of temperature between 330 and 430° C. and indicated by the DTG peak at ˜360-370° C. with a total weight loss of 65-80%, was due to degradation of the ester groups.

TABLE 31
Temperature of degradation at 1, 5 and 10% weight loss (T_{1%}^{d}, T_{5%}^{d},
T_{10%}^{d}, respectively), DTG peak temperatures (T_{D}), and extrapolated onset (Tₒₙ)
and offset (T_{off}) temperatures of degradation of LF-PMTAG Polyol Foams

| | Temperature (° C.) | | | | | | | | Weight loss (%) at | | | |
|---|---|---|---|---|---|---|---|---|---|---|---|---|
| Rigid LF-PMTAG | | | | | | | | | | | | |
| Polyol Foams | | | | | | | | | | | | |
| | T_{1%}^{d} | T_{5%}^{d} | T_{10%}^{d} | Tₒₙ | T_{D1} | T_{D2} | T_{D3} | T_{off} | Tₒₙ | T_{D1} | T_{D2} | T_{D3} |
| LF(D1) | 216 | 265 | 285 | 251 | 302 | 361 | 462 | 494 | 3 | 16 | 45 | 67 |
| LF(D2) | 178 | 254 | 274 | 254 | 302 | 364 | 467 | 496 | 5 | 21 | 44 | 69 |
| LF(S) | 209 | 253 | 274 | 249 | 296 | 361 | 446 | 491 | 4 | 18 | 39 | 66 |
| Flexible LF-PMTAG | | | | | | | | | | | | |
| Polyol Foams | | | | | | | | | | | | |
| | T_{1%}^{d} | T_{5%}^{d} | T_{10%}^{d} | Tₒₙ | T_{D1} | T_{D2} | T_{D3} | T_{off} | Tₒₙ | T_{D1} | T_{D2} | T_{D3} |
| LF(D1) | 205 | 249 | 278 | 250 | 307 | 362 | 450 | 486 | 5 | 17 | 44 | 68 |
| LF(D2) | 205 | 256 | 283 | 255 | 305 | 372 | | 457 | 5 | 17 | 42 | 77 |
| LF(S) | 206 | 248 | 275 | 246 | 301 | 367 | 468 | 489 | 5 | 17 | 58 | 78 |

### Thermal Transition Behavior of LF-PMTAG Polyol Foams

Typical curves obtained from the modulated DSC during the second heating cycle of the rigid and flexible LF-PMTAG Polyol foams are shown in FIGS. 36A and 36B, respectively. Table 32 lists the glass transition temperature (T_{g}) of the foams produced. Note that the glass transition as detected by DSC was broad and faint, and that the rigid foam obtained from the solvent fractionation (LF(S)-RF) did not show a T in the range of temperatures studied.

TABLE 32
Glass transition temperature (T_{g}, °
C.) of LF-PMTAG Polyol foams (2^{nd} heating)

| Rigid Foams | | Flexible Foams | |
|---|---|---|---|
| LF(D1)-RF166 | −32.1 | LF(D1)-FF155 | −26.8 |
| LF(D2)-RF165 | −13.2 | LF(D2)-FF155 | −14.2 |
| LF(S)-RF166 | — | LF(S)-RF155 | 31.8 |

### Compressive Strength of Rigid LF-MTAG Polyol Foams

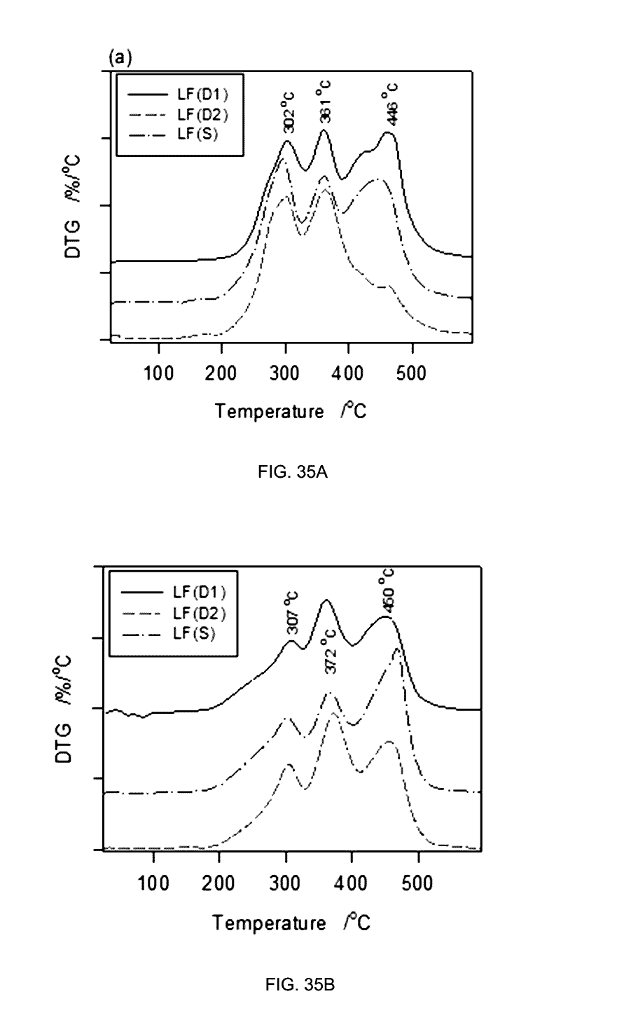

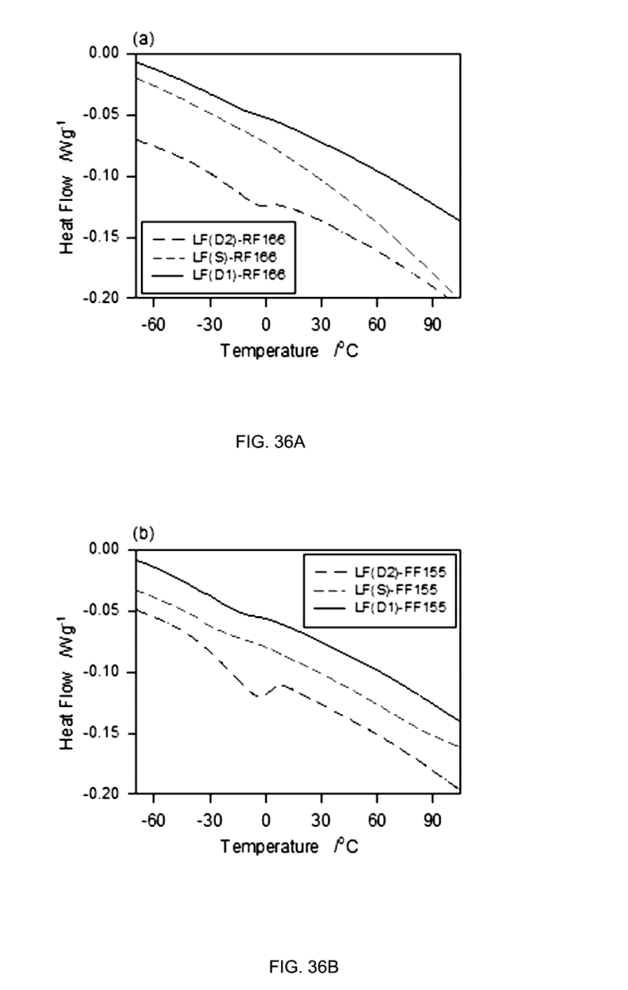

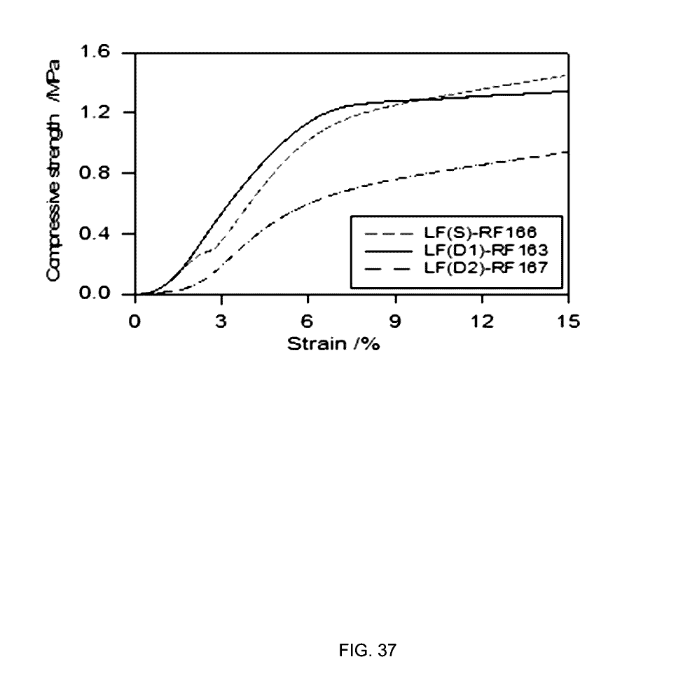

The strength of the foams were characterized by the compressive stress-strain measurements. Stress strain curves of the rigid LF(D1)-, LF(D2)- and LF(S)-PMTAG Polyol foams are shown in FIG. 37. The compressive strength values at 5, 10 and 15% deformation for the rigid foams are listed in Table 33.

TABLE 34
Compressive strength of rigid LF-PMTAG Polyol
foams at 5, 6, 10 and 15% deformation¹

| | Density | Compressive Strength (MPa) @ strain (%) | | | |
|---|---|---|---|---|---|
| Strain (%) | (kgm⁻³) | 5 | 6 | 10 | 15 |
| LF(D1)-RF163 | 163 | 1.07 | 1.19 | 1.29 | 1.35 |
| LF(D2)-RF167 | 167 | 0.50 | 0.60 | 0.80 | 0.94 |
| LF(S)-RF166 | 166 | 0.84 | 1.02 | 1.29 | 1.45 |

¹LF(D1)-RF163: LF(D1) Rigid LF(D1)-PMTAG Polyol Foam with density = 163 kgm⁻³; LF(D2)-RF167: Rigid LF(D2)-PMTAG Polyol Foam with density = 167 kgm⁻³; LF(S)-RF166: Rigid LF(S)-PMTAG Polyol Foam with density = 166 kgm⁻³

The compressive strength is highly dependent on the cellular structure of the foam. In the case of the rigid MTAG Polyol foams, the high mechanical strength of the foams was due to compact and closed cells as shown in FIGS. 32A-32F. The cell density of Rigid LF(D1)-PMTAG Polyol Foam and Flexible LF(D1)-PMTAG Polyol Foam from the SEM micrographs is ˜30 and 21 cell/mm², respectively. The cell density of Rigid LF(D2)-PMTAG Polyol Foam and Flexible LF(D2)-PMTAG Polyol Foam from the SEM micrographs is ˜10 and 18 cell/mm², respectively. The cell density of Rigid LF(S)-PMTAG Polyol Foam and Flexible LF(S)-PMTAG Polyol Foam from the SEM micrographs is ˜32 and 20 cell/mm², respectively. The elongation of the cells are due to the direction of rise and the boundaries caused by the walls of the cylindrical mold.

### Compressive Strength of Flexible PMTAG Polvol Foams

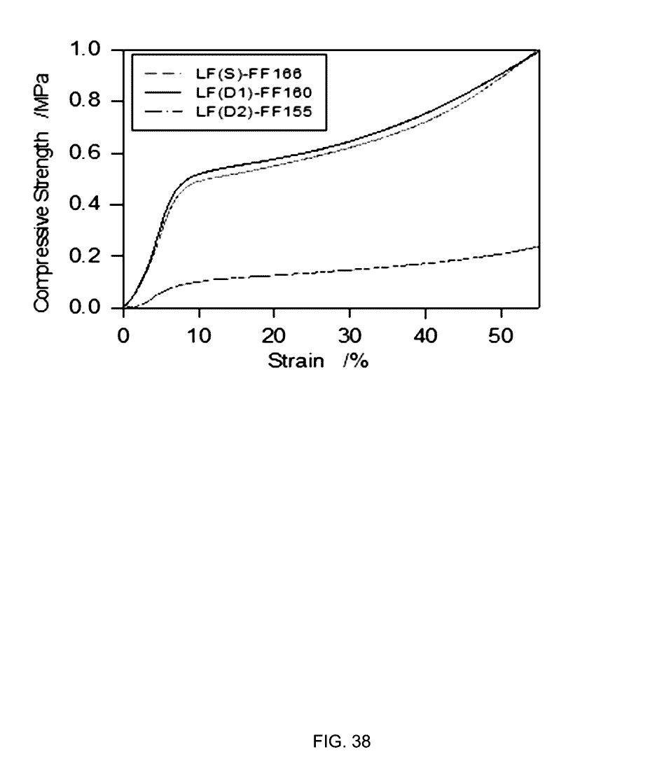

Stress strain curves of the flexible LF(D1)-, LF(D1)- and LF(S)-PMTAG Polyol foams produced using crude MDI are shown in FIG. 38. Table 34 lists the compressive strength at 10, 25 and 50% deformation of the flexible LF-PMTAG Polyol foams. As can be seen in FIG. 38, the compressive strength of the flexible LF(D1)-PMTAG Polyol foam was higher than flexible LF(S)-PMTAG Polyol foam due to higher density. The compressive strength of both is much higher than Flexible LF(D1)-PMTAG Polyol foam because the latter was prepared without solvent.

TABLE 34
Compressive strength values at 10, 25 and 50%
deformation of flexible LF-PMTAG Polyol foams

| | Density | Compressive Strength (MPa) @ Strain (%) | | |
|---|---|---|---|---|
| Strain (%) | (kgm⁻³) | 10 | 25 | 50 |
| LF(D1)-FF160 | 160 | 0.52 | 0.61 | 0.91 |
| LF(D2)-FF160 | 160 | 0.10 | 0.14 | 0.21 |
| LF(S)-FF155 | 155 | 0.49 | 0.58 | 0.89 |

¹LF(D1)-FF160: Flexible LF(D1)-PMTAG Polyol Foam with density=160 kgm⁻³; LF(D2)-FF160: Flexible LF(D2)-PMTAG Polyol Foam with density=160 kgm⁻³; LF(S)-FF155: Flexible LF(S)-PMTAG Polyol Foam with density=155 kgm⁻³;

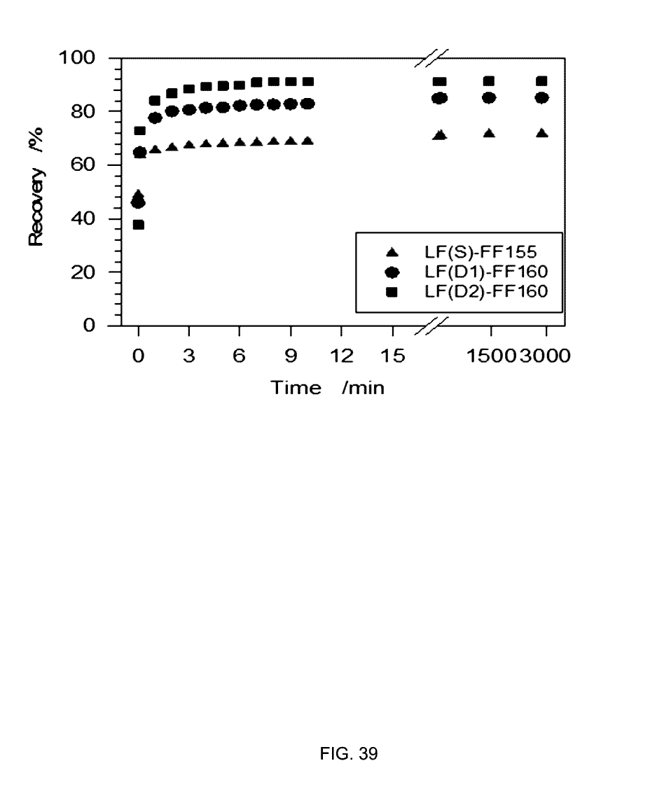

FIG. 39 shows the percentage of recovery of flexible LF-PMTAG Polyol foams as a function of time. Table 35 lists the recovery values after 48 hours. Note that flexible LF(S)-, LF(D1)- and LF(D1)-PMTAG Polyol foams recovered ˜70, 85 and 91% of their initial thickness after 1 hour.

TABLE 35
Recovery (%) values of LF(D)-FF160
and LF(S)-FF155 after 48 hours¹

| | Density | Recovery |
|---|---|---|
| Foam | (kg/m³) | (%) |
| LF(D1)-FF160 | 160 | 85 |
| LF(D2)-FF160 | 160 | 91 |
| LF(S)-FF155 | 155 | 72 |

¹LF(D1)-FF160: Flexible LF(D1)-PMTAG Polyol Foam with density = 160 kgm⁻³; LF(D2)-FF160: Flexible LF(D2)-PMTAG Polyol Foam with density = 166 kgm⁻³; LF(S)-FF155: Flexible LF(S)-PMTAG Polyol Foam with density = 155 kgm⁻³;

### Waxes and Cosmetics

In certain aspects, the disclosure provides wax compositions, which includes polyester polyols made by the methods of any of the foregoing aspects and embodiments, or which is derived from a polyester polyol made by the methods of any of the foregoing aspects and embodiments.

In certain aspects, the disclosure provides personal care compositions, such as cosmetics compositions, which includes polyester polyols made by the methods of any of the foregoing aspects and embodiments, or which is derived from a polyester polyol made by the methods of any of the foregoing aspects and embodiments.

The foregoing detailed description and accompanying figures have been provided by way of explanation and illustration, and are not intended to limit the scope of the invention. Many variations in the present embodiments illustrated herein will be apparent to one of ordinary skill in the art, and remain within the scope of the invention and their equivalents.

1. A method of making a triacylglycerol polyol from palm oil, the method comprising:
providing a metathesized triacylglycerol composition, which is formed by the cross-metathesis of a natural oil with lower-weight olefins, and which comprises triglyceride compounds having one or more carbon-carbon double bonds;
separating a fraction of the metathesized triacylglycerol composition to form a fractionated metathesized triacylglycerol composition, which comprises compounds having one or more carbon-carbon double bonds; and
reacting at least a portion of the carbon-carbon double bonds in the compounds comprised by the fractionated metathesized triacylglycerol composition to form a triacylglycerol polyol composition.

2. The method of claim 1, wherein the lower-weight olefins comprise C₂-C₆ olefins.

3. The method of claim 1, wherein the lower-weight olefins comprise C₂-C₆ alpha olefins.

4. The method of claim 3, wherein the lower-weight olefins comprise ethylene or 1-butene.

5. The method of claim 4, wherein the lower-weight olefins comprise 1-butene.

6. The method of claim 1, wherein the natural oil comprises canola oil, soybean oil, palm oil, or a combination thereof.

7. The method of claim 1, wherein the metathesized triacylglycerol composition comprises triglycerides that comprise 9-decenoate residues.

8. The method of claim 1, wherein the metathesized triacylglycerol composition comprises triglycerides that comprise 9-dodecenoate residues.

9. The method of claim 1, wherein the separating comprises:
melting the metathesized triacylglycerol composition;
cooling the melted metathesized triacylglycerol composition to form a metathesized triacylglycerol composition having a liquid phase and a solid phase; and
separating at least a portion of the liquid phase to form the fractionated metathesized triacylglycerol composition.

10. The method of claim 1, wherein the separating comprises:
melting the metathesized triacylglycerol composition;
cooling the melted metathesized triacylglycerol composition to form a metathesized triacylglycerol composition having a liquid phase and a solid phase; and
separating at least a portion of the solid phase to form the fractionated metathesized triacylglycerol composition.

11. The method of claim 1, wherein the separating comprises:
dissolving the metathesized triacylglycerol composition in a solvent composition;
cooling the dissolved metathesized triacylglycerol composition to crystallize a portion of the metathesized triacylglycerol composition; and
separating at least a portion of the dissolved metathesized triacylglycerol composition from the crystallized metathesized triacylglycerol composition to form the fractionated metathesized triacylglycerol composition.

12. The method of claim 1, wherein the separating comprises:
dissolving the metathesized triacylglycerol composition in a solvent composition;
cooling the dissolved metathesized triacylglycerol composition to crystallize a portion of the metathesized triacylglycerol composition; and
separating at least a portion of the crystallized metathesized triacylglycerol composition from the dissolved metathesized triacylglycerol composition to form the fractionated metathesized triacylglycerol composition.

13. (canceled)

14. The method of claim 1, wherein the fractionated metathesized triacylglycerol composition has an iodine value that is greater than that of the metathesized triacylglycerol composition.

15. The method of claim 1, wherein the fractionated metathesized triacylglycerol composition has an iodine value that is less than that of the metathesized triacylglycerol composition.

16-19. (canceled)

20. The method of claim 1, wherein the reacting comprises epoxidizing at least a portion of the carbon-carbon double bonds in the compounds comprised by the fractionated metathesized triacylglycerol composition to form a triacylglycerol polyol, followed by hydroxylating at least a portion of the epoxide groups formed by the epoxidizing step.

21. The method of claim 20, wherein the epoxidizing comprises reacting at least a portion of the carbon-carbon double bonds in the compounds comprised by the metathesized fractionated triacylglycerol composition with a peroxyacid.

22-24. (canceled)

25. The method of claim 20, wherein the reacting further comprises, after the epoxidizing and before the hydroxylating, neutralizing the product of the epoxidizing step.

26. The method of claim 20, wherein the epoxidizing comprises reacting at least a portion of the carbon-carbon double bonds in the compounds comprised by the metathesized triacylglycerol composition with formic acid or acetic acid.

27. The method of claim 20, wherein the hydroxylating comprises reacting at least a portion of the epoxide groups formed by the epoxidizing with perchloric acid.

28. A method of forming a polyurethane composition, comprising:
providing a triacylglycerol polyol and an organic diisocyanate, wherein providing the triacylglycerol polyol comprises making a triacylglycerol polyol according to claim 1; and
reacting the triacylglycerol polyol and the organic diisocyanate to form a polyurethane composition.

29-32. (canceled)
